(19) United States
(12) Patent Application Publication
(10) Pub. No.: US 2015/0259371 A1
(43) Pub. Date: Sep. 17, 2015

(54) METHODS AND COMPOUNDS FOR INHIBITING GLYCOSYLTRANSFERASES

(71) Applicants: David Jaro Vocadlo, Burnaby (CA); Tracey Maureen Gloster, Burnaby (CA); Lehua Deng, Burnaby (CA); Wesley Franklin Zandberg, Deroche (CA)

(72) Inventors: David Jaro Vocadlo, Burnaby (CA); Tracey Maureen Gloster, Burnaby (CA); Lehua Deng, Burnaby (CA); Wesley Franklin Zandberg, Deroche (CA)

(73) Assignee: SIMON FRASER UNIVERSITY, Burnaby, BC (CA)

(21) Appl. No.: 13/696,248

(22) PCT Filed: May 6, 2011
(86) PCT No.: PCT/CA2011/000538

§ 371 Date: Mar. 4, 2013

Related U.S. Application Data
(60) Provisional application No. 61/331,912, filed on May 6, 2010.

(51) Int. Cl. C07H 15/203; C07H 15/04

(52) U.S. Cl. CPC C07H 15/203; C07H 15/04

(57) ABSTRACT

The invention provides, in part, compounds that are capable of inhibiting a glycosyltransferase, such as a uridine diphospho-N-acetylglucosamine:polypeptide β N-acetylglucosaminyltransferase, an N-acetylgalactosaminyltransferase, a fucosyltransferase, a xylotransferase or a sialyltransferase. The invention also provides methods for using the compounds.

### FIELD OF INVENTION

This application relates to compounds which inhibit glycosyltransferases and uses thereof.

### BACKGROUND OF THE INVENTION

Glycosyltransferases (GTs) are ubiquitous enzymes responsible for the biosynthesis of myriad glycoconjugates. The formation of these glycoconjugates is generally mediated by GT-catalyzed transfer of a sugar residue from an anionic nucleotide sugar donor to various acceptors molecules, which may be proteins, lipids, saccharides, or metabolites. Mammals have over 200 GTs that give rise to diverse glycoconjugates and these structures are emerging as regulators of quality control, cellular structure, signaling, gene transcription, and intercellular communication¹.

The post-translational installation of single 2-acetamido-2-deoxy-D-glucopyranose (GlcNAc) residues β-glycosidically linked to serine or threonine residues of proteins (O-GlcNAc)² is a nuclear and cytoplasmic modification ubiquitously found in multicellular eukaryotes. Several hundred proteins, involved in a wide range of cellular functions, have been shown to be modified³. O-GlcNAc can be installed and removed several times during the lifetime of a given protein and its cycling is regulated by two enzymes; a GT (uridine diphospho-N-acetylglucosamine:polypeptide β-N-acetylglucosaminyltransferase; or “OGT”)³, which transfers O-GlcNAc onto proteins, and a glycoside hydrolase (O-GlcNAcase; or “OGA”)³ that removes O-GlcNAc (FIG. 1A). O-GlcNAc has been proposed to mediate cellular processes in nutrient signaling, regulation of gene transcription, cellular stress response, and cell division³. The donor substrate used by OGT, uridine diphospho-N-acetylglucosamine (UDP-GlcNAc), is biosynthesized through the action of enzymes comprising the hexosamine biosynthetic pathway (HBP) (FIG. 1B).

A number of chemical approaches have been used to reduce O-GlcNAc levels. For example, 6-diazo-5-oxo-L-norleucine and L-azaserine block UDP-GlcNAc biosynthesis but these compounds inhibit other amidotransferases including those involved in purine and pyrimidine biosynthesis, leading to DNA damage⁴; and alloxan is a uracil analogue that is toxic and affects many cellular processes⁵. OGT inhibitors have also been recently discovered by library screening⁶ but have not been well characterized in cells.

Selecting proteins recognize cell surface ligands that are carbohydrate structures. This recognition process mediates the rolling of leukocytes and other cell lineages, including cancer cells, along endothelial surfaces, which serves as the first step to the extravasation and invasion of these cells into the adjacent tissues. The three known selectins (P-selectin (CD62P), L-selectin (CD62L), and E-selectin (CD62E) bind specific carbohydrate epitopes present on cell surface biomolecules including lipids and proteins⁶²⁻⁶⁵. These carbohydrate structures include sialyl Lewis A (sLe^{a}) and sialyl Lewis X (sLe^{X})⁶²⁻⁶⁵. The biosynthesis of these carbohydrate structures is mediated by a number of glycosyltransferases including, as a last step, the attachment of a fucose residue onto N-acetylglucosamine. This last step is catalyzed by enzymes known as fucosyltransferases (FUT). There are several different fucosyltransferases known in humans and various enzymes as reviewed in the scientific literature are involved in the synthesis of these carbohydrate structures^{62-64,66,67}. Increased levels of these carbohydrate structures permits cancer cells and leukocytes to bind more efficiently to selectins. These structures are therefore considered mediators of these processes that can initiate the attachment of circulating cancer cells to tissues and thereby enable metastasis⁶⁶⁻⁶⁸. Likewise, increased expression of these carbohydrate structures can enable extravasation of leukocytes into tissues and thereby contribute to undesirable inflammatory processes^{63,64,69,70}. Studies in mice deficient in fucosyltransferase IV or VII showed decreased number of rolling leukocytes as well as a higher leukocyte rolling velocity^{63,64,71,72}. Likewise, in several types of cancer some of these carbohydrate structures have been found to correlate with a poor outcome⁶⁶⁻⁶⁸. Further, some fucosyltransferases have been shown to be elevated in cancer tissues⁶⁶⁻⁶⁸. Accordingly, there have been attempts to design selectin antagonists based on the structure of sLex as potential therapeutics⁶². Fucosyltransferases and fucose containing glycan structures have been implicated in a number of other diseases including Helicobacter pylori infections⁷, glaucoma⁶⁵, and atherosclerosis⁷⁰. Recently, the recognition that therapeutic antibodies lacking fucose have been shown to have potential benefit⁷³.

Biosynthetic pathways exist within cells and tissues to generate a range of nucleotide sugar donors such as UDP-GlcNAc, UDP-GalNAc, CMP-sialic acid, UDP-Xyl, and GDP-Fuc⁸. Furthermore, biosynthetic pathways, known as salvage pathways⁸, enable the assimilation and conversion of exogenously added sugars, such as GlcNAc⁹, GlcNH₂¹⁰, GalNAc¹¹, ManNAc^{12,13}, and Fuc^{14,15}, into the respective nucleotide sugars UDP-GlcNAc, UDP-GlcNAc, UDP-GalNAc or UDP-GlcNAc, CMP-sialic acid, and GDP-fucose. The enzymes in the salvage pathways and the biosynthetic pathways used by cells (including bacterial cells) to generate these nucleotide sugar donors are capable of enabling cells and living organisms to assimilate various sugar analogues to form the nucleotide sugar donors^{9,16-21}.

A number of sugar analogues have been synthesized ²²⁻²⁸. Nucleotide sugar analogues in which the endocyclic sugar ring is replaced by sulphur appear to be poor substrates of GTs²⁹⁻³¹ and are turned over at only approximately 0.2 to 5% the rate as compared to the naturally occurring nucleotide sugar²⁹⁻³¹. In other cases when the endocylic oxygen of the sugar ring is replaced by carbon the nucleotide sugar analogue does not appear to be turned over at all³². Derivatized metabolic intermediates, such as sugar-1-phosphates, can diffuse across the membrane and thereby be delivered to tissues where they are processed and assimilated into the biosynthetic pathways to generate the corresponding nucleotide sugar³³.

### SUMMARY OF THE INVENTION

The invention provides, in part, inhibitors of glycosyltransferases and uses thereof

In one aspect, the invention provides a method of inhibiting a glycosyltransferase (GT) in a subject in need thereof, the method comprising administering to the subject an effective amount of a compound of any one of Formula (I-X) or a pharmaceutically acceptable salt thereof:

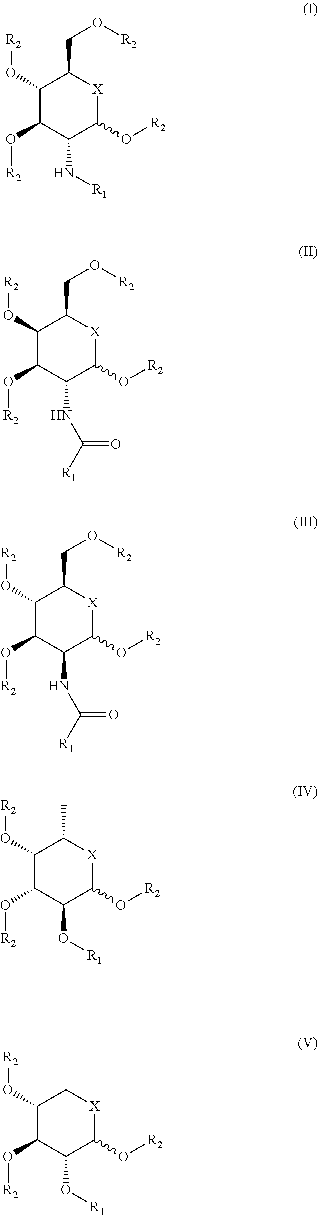

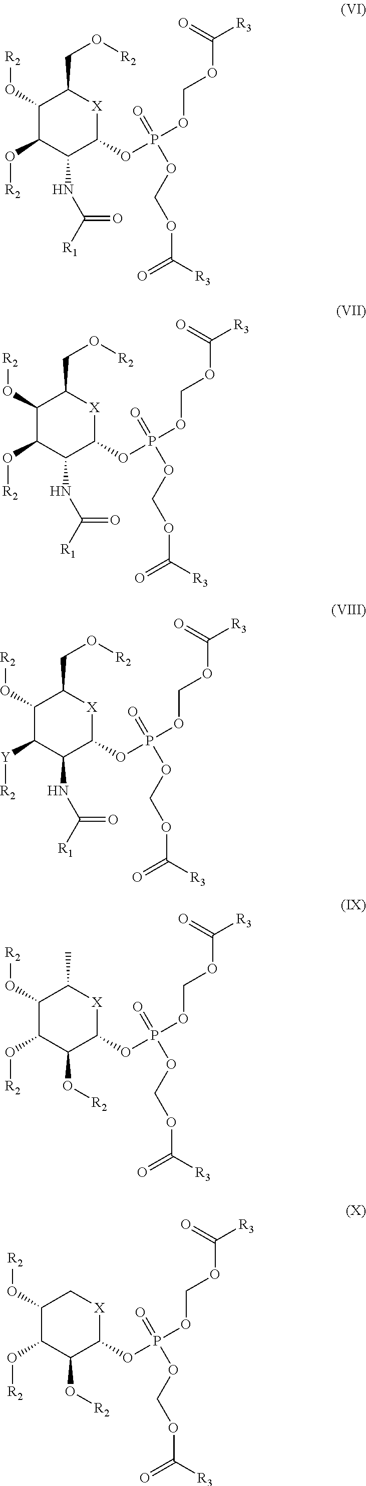

wherein X where present may be S, Se or CH₂; Y where present may be S or Se; R₁ may be either H or C(O)R₄, wherein R₄ may be H, CH₂OH, CH₂N₃, CH₂SH, small branched or unbranched alkyl, ether, thioether, urea, or thiourea, wherein the alkyl, ether, thioether, urea, or thiourea are optionally substituted; R₂ is H or C(O)R₅, wherein R₅ may be optionally substituted alkyl, alkenyl, alkynyl, aryl, or heteroaryl; R₃ where present may be H or C(O)R₆, wherein R₆ may be optionally substituted alkyl, alkenyl, alkynyl, aryl, or heteroaryl; and [symbol] may be the alpha anomer, the beta anomer, or both.

In an alternative aspect, the invention provides a method of reducing the level of a nucleotide sugar-modified biomolecule in a sample or in a subject in need thereof, the method comprising contacting the sample with, or administering to the subject, an effective amount of a compound of any one of Formula (I-X) or a pharmaceutically acceptable salt thereof:

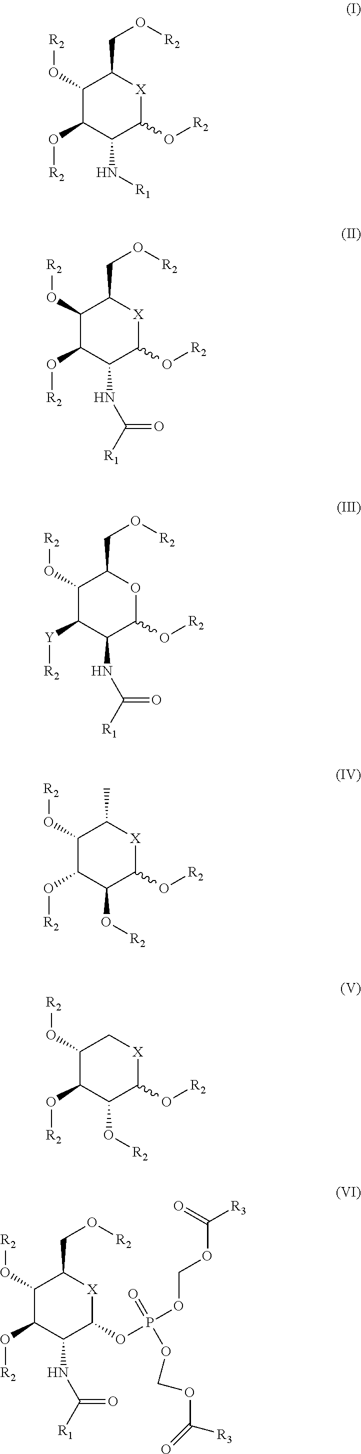

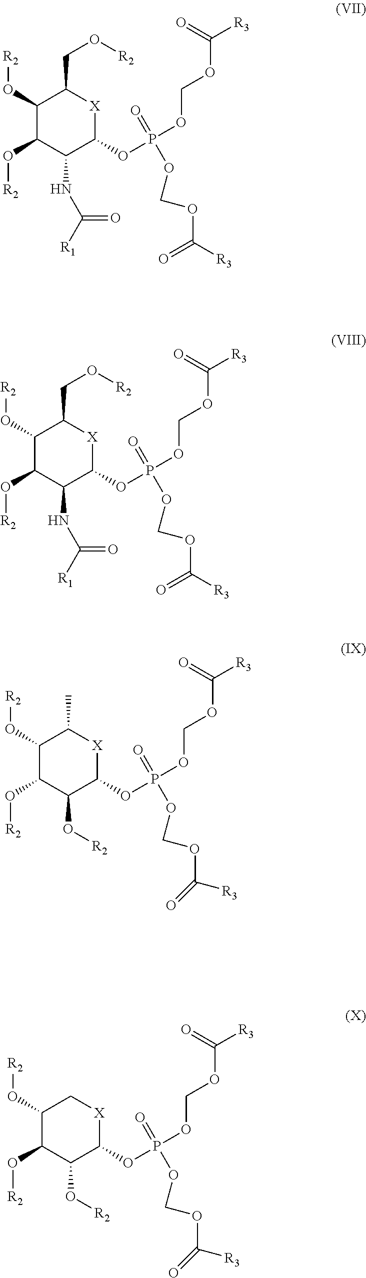

wherein X where present may be S, Se or CH₂; Y where present may be S or

Se; R₁ may be either H or C(O)R₄, wherein R₄ may be H, CH₂OH, CH₂N₃, CH₂SH, small branched or unbranched alkyl, ether, thioether, urea, or thiourea, wherein the alkyl, ether, thioether, urea, or thiourea are optionally substituted; R₂ is H or C(O)R₅, wherein R₅ may be optionally substituted alkyl, alkenyl, alkynyl, aryl, or heteroaryl; R₃ where present may be H or C(O)R₆, wherein R₆ may be optionally substituted alkyl, alkenyl, alkynyl, aryl, or heteroaryl; and [symbol] may be the alpha anomer, the beta anomer, or both.

In an alternative aspect, the invention provides a method of treating a condition that is modulated by a GT in a subject in need thereof, the method comprising administering to the subject an effective amount of a compound of any one of Formula (I-X) or a pharmaceutically acceptable salt thereof:

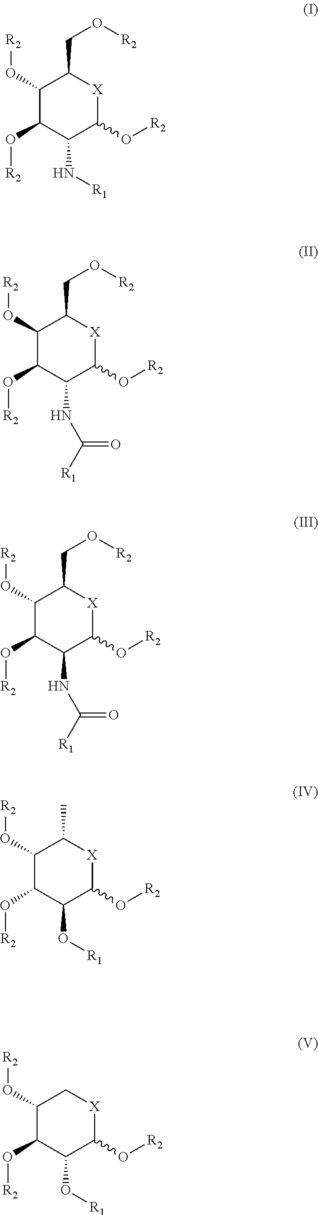

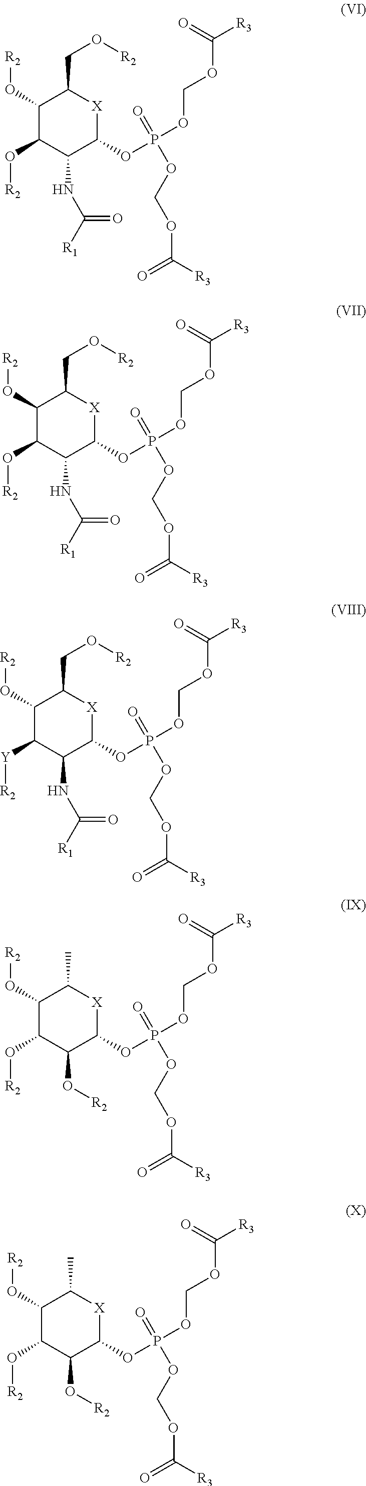

wherein X where present may be S, Se or CH₂; Y where present may be S or Se; R₁ may be either H or C(O)R₄, wherein R₄ may be H, CH₂OH, CH₂N₃, CH₂SH, small branched or unbranched alkyl, ether, thioether, urea, or thiourea, wherein the alkyl, ether, thioether, urea, or thiourea are optionally substituted; R₂ is H or C(O)R₅, wherein R₅ may be optionally substituted alkyl, alkenyl, alkynyl, aryl, or heteroaryl; R₃ where present may be H or C(O)R₆, wherein R₆ may be optionally substituted alkyl, alkenyl, alkynyl, aryl, or heteroaryl; and [symbol] may be the alpha anomer, the beta anomer, or both.

In an alternative aspect, the invention provides a method of inhibiting a GT in a cell or tissue, the method comprising administering to the cell or tissue an effective amount of a compound of any one of Formula (I-X) or a pharmaceutically acceptable salt thereof:

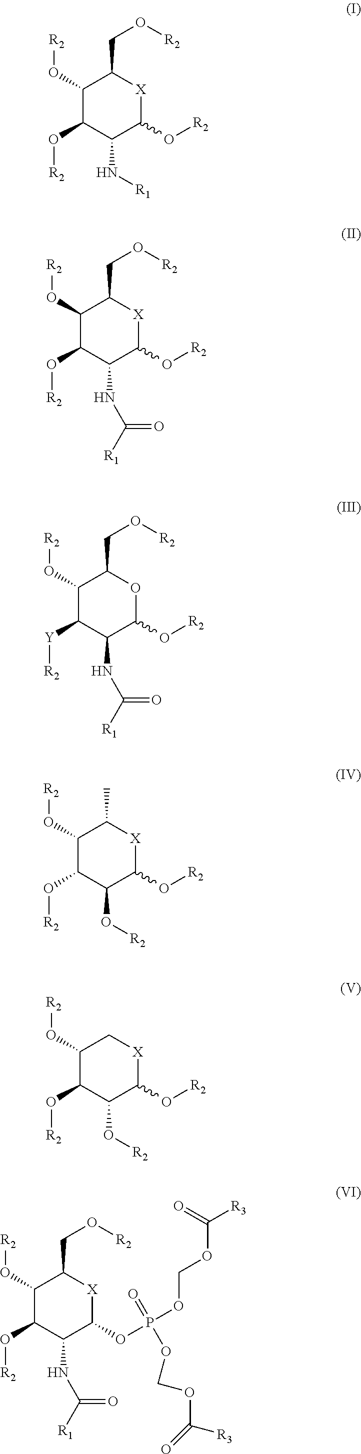

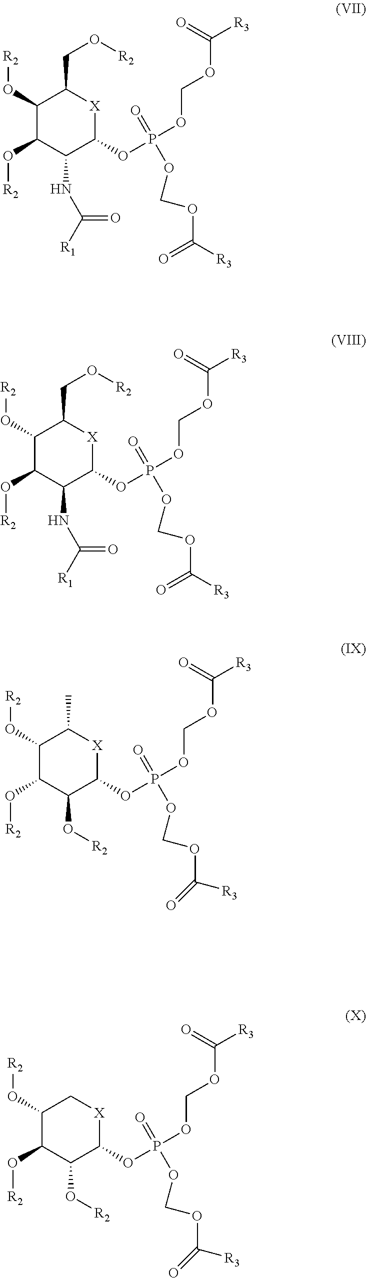

wherein X where present may be S, Se or CH₂; Y where present may be S or Se; R₁ may be either H or C(O)R₄, wherein R₄ may be H, CH₂OH, CH₂N₃, CH₂SH, small branched or unbranched alkyl, ether, thioether, urea, or thiourea, wherein the alkyl, ether, thioether, urea, or thiourea are optionally substituted; R₂ is H or C(O)R₅, wherein R₅ may be optionally substituted alkyl, alkenyl, alkynyl, aryl, or heteroaryl; R₃ where present may be H or C(O)R₆, wherein R₆ may be optionally substituted alkyl, alkenyl, alkynyl, aryl, or heteroaryl; and [symbol] may be the alpha anomer, the beta anomer, or both.

In alternative aspects, the invention provides the use of a compound of Formula (I-X) or a pharmaceutically acceptable salt thereof:

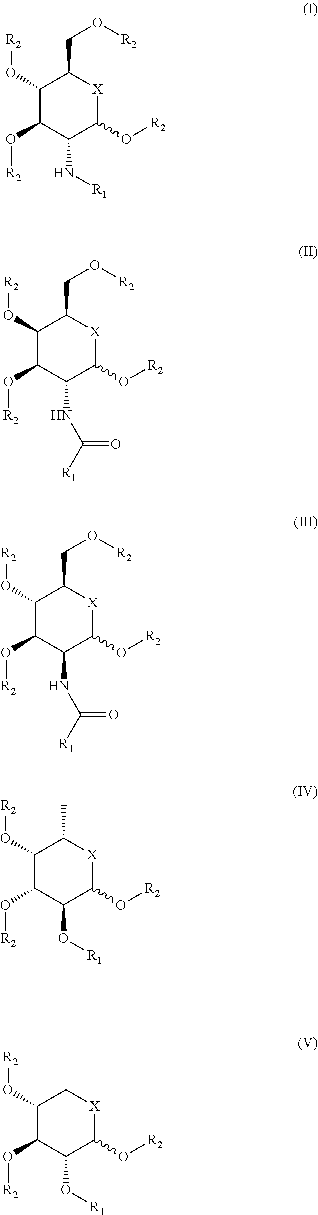

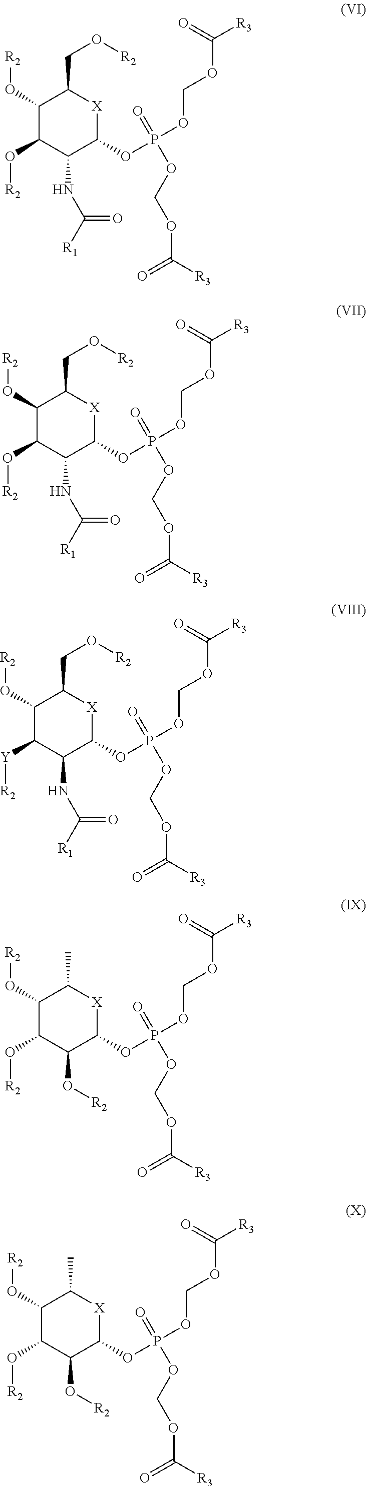

wherein X where present may be S, Se or CH₂; Y where present may be S or Se; R₁ may be either H or C(O)R₄, wherein R₄ may be H, CH₂OH, CH₂N₃, CH₂SH, small branched or unbranched alkyl, ether, thioether, urea, or thiourea, wherein the alkyl, ether, thioether, urea, or thiourea are optionally substituted; R₂ is H or C(O)R₅, wherein R₅ may be optionally substituted alkyl, alkenyl, alkynyl, aryl, or heteroaryl; R₃ where present may be H or C(O)R₆, wherein R₆ may be optionally substituted alkyl, alkenyl, alkynyl, aryl, or heteroaryl; and [symbol] may be the alpha anomer, the beta anomer, or both, for inhibiting a GT, reducing the level of a nucleotide sugar-modified biomolecule , or treating a condition that is modulated by a GT, in a subject in need thereof, or in a sample, cell or tissue.

In alternative aspects, the invention provides a compound of Formula (I-X) or a pharmaceutically acceptable salt thereof:

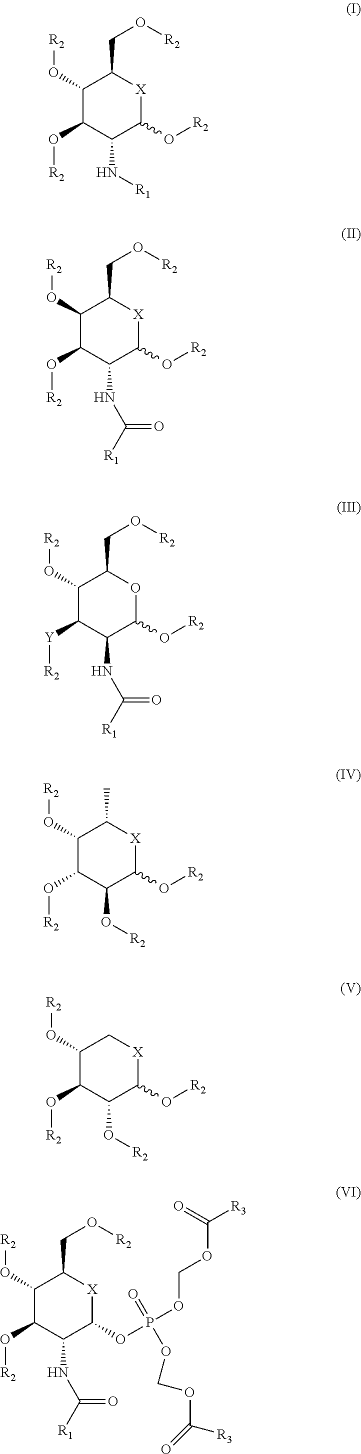

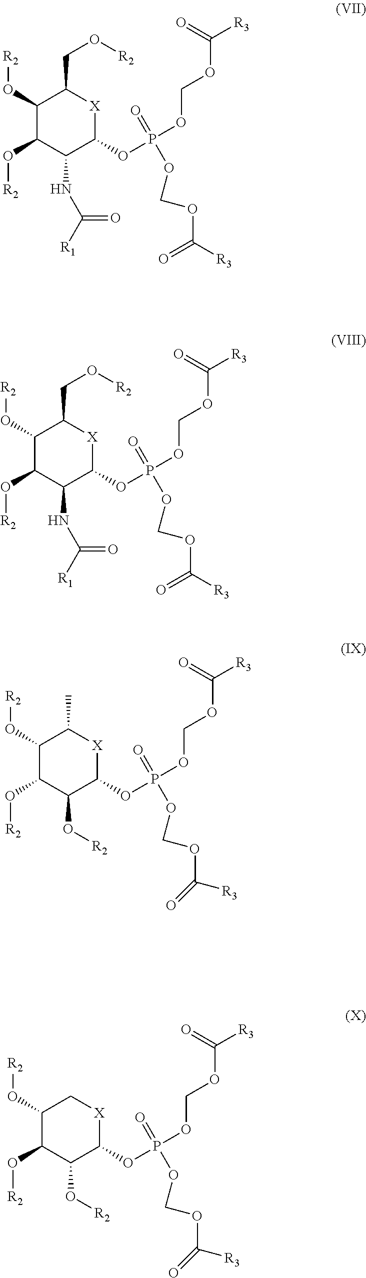

wherein X where present may be S, Se or CH₂; Y where present may be S or Se; R₁ may be either H or C(O)R₄, wherein R₄ may be H, CH₂OH, CH₂N₃, CH₂SH, small branched or unbranched alkyl, ether, thioether, urea, or thiourea, wherein the alkyl, ether, thioether, urea, or thiourea are optionally substituted; R₂ is H or C(O)R₅, wherein R₅ may be optionally substituted alkyl, alkenyl, alkynyl, aryl, or heteroaryl; R₃ where present may be H or C(O)R₆, wherein R₆ may be optionally substituted alkyl, alkenyl, alkynyl, aryl, or heteroaryl; and [symbol] may be the alpha anomer, the beta anomer, or both, for inhibiting a GT, reducing the level of a nucleotide sugar-modified biomolecule, or treating a condition that is modulated by a GT, in a subject in need thereof, or in a sample, cell or tissue, wherein optionally said compound is not 5SGlcNAc, Ac-5SGlcNAc, 5CH₂GlcNAc, UDP-5CH₂GlcNAe, 5SGlNAz, Ac-5SGlcNAz, or 5-T-Fuc.

In alternative aspects, the invention provides a kit comprising a composition or compound according to the inveniton together with instructions for use in for inhibiting a GT, reducing the level of a nucleotide sugar-modified biomolecule, or treating a condition that is modulated by a GT.

In alternative embodiments, the subject may be a human. In some embodiments, the small alkyl may be methyl, ethyl, propyl, butyl, isopropyl, isobutyl, valeryl, or isovaleryl; the ether may be O-methyl, O-ethyl, O-propyl, O-isopropyl, O-butyl, or O-isobutyl; the thioether may be S-methyl, S-ethyl, S-propyl, S-isopropyl, S-butyl, or S-isobutyl; one, two, three or four of R₂ may be C(O)CH₃.

In some embodiments, the GT may be a fucosyltransferase (FUT), and the method may comprise administering an effective amount of a compound of Formula (IV) or (IX) or a pharmaceutically acceptable salt thereof.

In some embodiments, the GT may be a fucosyltransferase (FUT), and the method may comprise contacting a sample or cell comprising an antibody with an effective amount of a compound of Formula (IV) or (IX) or a pharmaceutically acceptable salt thereof.

In some embodiments, the GT may be a fucosyltransferase (FUT), and the method may comprise administering to the subject an effective amount of a compound of Formula (IV) or (IX) or a pharmaceutically acceptable salt thereof.

In some embodiments, the condition may be inflammation, an autoimmune disorder or a cancer.

In some embodiments, the GT may be a fucosyltransferase (FUT), and the method may comprises contacting the cell or tissue with an effective amount of a compound of Formula (IV) or (IX) or a pharmaceutically acceptable salt thereof.

In some embodiments, the compound may be 5-T-Fuc.

In some embodiments, the GT may be uridine diphospho-N-acetylglucosamine:polypeptide O-N-acetylglucosaminyltransferase (OGT), and the method may comprise administering an effective amount of a compound of Formula (I) or (VI) or a pharmaceutically acceptable salt thereof.

In some embodiments, the GT may be OGT, and the method may comprise administering to the subject an effective amount of a compound of Formula (I) or (VI) or a pharmaceutically acceptable salt thereof.

In some embodiments, the condition may be cancer, diabetes, complications of diabetes, inflammation, or autoimmune disease.

In some embodiments, the GT may be OGT and the method may comprise contacting the cell or tissue with an effective amount of a compound of Formula (I) or (VI) or a pharmaceutically acceptable salt thereof.

In some embodiments, the cell may be a cancer cell or the tissue may be pancreatic tissue.

In some embodiments, the compound may increase the level of the OGT. In some embodiments, the compound may reduce the level of an O-GlcNAcase.

In some embodiments, the X may be S, R₂ may be H or C(O)CH₃, and R₄ may be H, CH₂N₃, CH₃, CH₂CH₃, (CH₂)₂CH₃, CH(CH₃)₂, (CH₂)₃CH₃, CH₂CH(CH₃)₂, (CH₂)₄CH₃, C(CH₃)₃, CH₂C₆H₁₁, CH₂C₁₀H₈, CH₂CH(CH₃)CH₂CH₃, or (CH₂)₃CH(CH₃)₂.

This summary of the invention does not necessarily describe all features of the invention.

### BRIEF DESCRIPTION OF THE DRAWINGS

These and other features of the invention will become more apparent from the following description in which reference is made to the appended drawings wherein:

FIGS. 1A-B are schematic diagrams showing (A) dynamic cycling of the O-GlcNAc modification of nucleocytoplasmic proteins in eukaryotes. The modification is modulated by two enzymes: OGT transfers O-GlcNAc onto proteins from the UDP-GlcNAc sugar donor and OGA catalyzes hydrolysis of the sugar moiety and (B) the hexosamine biosynthetic pathway leading to the biosynthesis of UDP-GlcNAc, where endocyclic heteroatom is denoted X (0 in nature and S in the synthetic compounds according to some embodiments).

FIG. 1C shows the structure of 5SGlcNAc, UDP-5SGlcNAc,pMP-5SGlcNAc, and Me-5SGlcNAc (X═O or S).

FIG. 1D shows in vitro enzymatic synthesis of UDP-5SGlcNAc catalyzed by the human enzymes of the HBP and monitored by capillary electrophoresis; the traces shows absorbance at 254 nm as a function of retention time. Upper trace shows the crude reaction mixture prior to purification and the lower trace shows UDP-5SGlcNAc following ion exchange and HPLC purification. Peak A corresponds to GDP-Glc (internal standard spiked into samples prior to analysis) and peak B corresponds to UDP-5SGlcNAc.

FIG. 1E shows inhibition of OGT-catalyzed transfer of O-GlcNAc onto nup62 by UDP-5SGlcNAc. The K, value, determined by Dixon analysis, is 8 μM.

FIG. 1F-G. NMR spectrum of UDP-SSGlcNAc. (F) ^{I}H NMR spectrum of UDP-5SGlcNAc. Peaks at 8.35, 3.10 and 1.18 ppm arise from the triethylammonium counterion. (G) Expansion of ¹H NMR spectrum of UDP-5SGlcNAc covering the region from 4.40 to 3.20 ppm.

FIG. 1H-I. NMR spectra of UDP-5SGlcNAc. (H) ¹³C {¹H} NMR spectrum of UDP-5SGlcNAc. Peaks at 8.1 (CH₃) and 46.6 (CH₂) are derived from the triethylammonium cation. The peak at 174.2 ppm is from the carbonyl of the acetyl group. (I) ³¹P{¹H} NMR spectrum of UDP-5SGlcNAc referenced to 85% H₃PO₄ at 0 ppm.

FIG. 1J-K. Two dimensional NMR spectra of UDP-5SGlcNAc. (K) HMQC spectrum and (L) COSY spectrum.

FIGS. 2A-L are a series of western blots showing that 5SGlcNAc acts in cells to decrease O-GlcNAc levels in a dose and time dependent manner.

FIG. 2A shows Western blots of COS-7 cell lysates following Ac-5SGlcNAc administration at different doses (0-1000 μM) for 24 h. Upper panel, probed with anti-O-GlcNAc antibody (CTD110.6); lower panel, probed with anti-actin antibody. The plot shows the densitometry analysis and yields an EC₅₀ value for reduction of O-GlcNAc levels of 5 μM.

FIG. 2B shows Western blots of COS-7 cell lysates following Ac-5SGlcNAc administration at 50 μM for different amounts of hours. Upper panel, probed with anti-O-GlcNAc antibody (CTD 110.6); lower panel, probed with anti-actin antibody. The plot shows the densitometry analysis as a function of dose; O-GlcNAc levels diminish to a low level by 24 h.

FIG. 2C shows Western blots of COS-7 cell lysates following Ac-5SGlcNAc administration at 50 μM for different amounts of days. Probed with (from top to bottom) anti-O-GlcNAc antibody (CTD 110.6), anti-actin antibody, anti-OGA antibody, and anti-OGT antibody.

FIG. 2D shows Western blots of COS-7 cell lysates administered with specified agents at 50 μM for 24 h; vehicle only (C), Ac-GlcNAc (G) or Ac-5SGlcNAc (5SG). Probed with (from top to bottom) anti-O-GlcNAc antibody (CTD 110.6), anti-actin antibody, anti-OGA antibody, and anti-OGT antibody.

FIG. 2E shows Western blot of immunoprecipitated nup62 from cell lysates following vehicle (C) or 250 μM Ac-5SGlcNAc (5SG) treatment for 24 h. Following immunoprecipitation, nup62 was incubated with UDP-GalNAz in the absence (−) or presence (+) of GalT1 and chemoselectively labeled. Upper panel, probed with anti-nup62 antibody; lower panel, probed with streptavidin.

FIG. 2F shows Western blot of immunoprecipitated nup62 from cell lysates following vehicle (C) or 250 μM Ac-5SGlcNAc (5SG) treatment for 24 h. Following immunoprecipitation, nup62 was incubated with buffer (−) or with BtGH84 glucosaminidase for 2 h to remove O-GlcNAc. Upper panel, probed with anti-nup62 antibody; lower panel, probed with anti-O-GlcNAc antibody (long exposure time shown at top and short exposure shown below).

FIG. 2G. Western blots of COS-7 cell lysates following 5SGlcNAc administration at different doses (0-5000 μM) for 24 h. Upper panel, probed with anti-O-GlcNAc antibody (CTD110.6); lower panel, probed with anti-actin antibody. The plot shows the densitometry analysis converted to a % O-GlcNAc modification (corrected for actin levels) relative to the untreated control sample as a function of dose; this gives an EC₅₀ of 700 _{μ}M.

FIG. 2H. Western blots of COS-7 cell lysates following no (C), Ac-GlcNAc (G) or Ac-5SGlcNAc (5SG) administration at 50 μM for 24 h. Probed with different anti-O-GlcNAc antibodies: CTD110.6, RL2 and HGAC85, from left to right.

FIG. 21. Western blots of COS-7 cell lysates following no (C), 50 μM Ac-5SGlcNAc treatment for 48 h (5SG), 50 μM Ac-5SGlcNAc treatment for 24 h, followed by no treatment for 24 h (W). Upper panel, probed with anti-O-GlcNAc antibody (CTD110.6); lower panel, probed with anti-actin antibody.

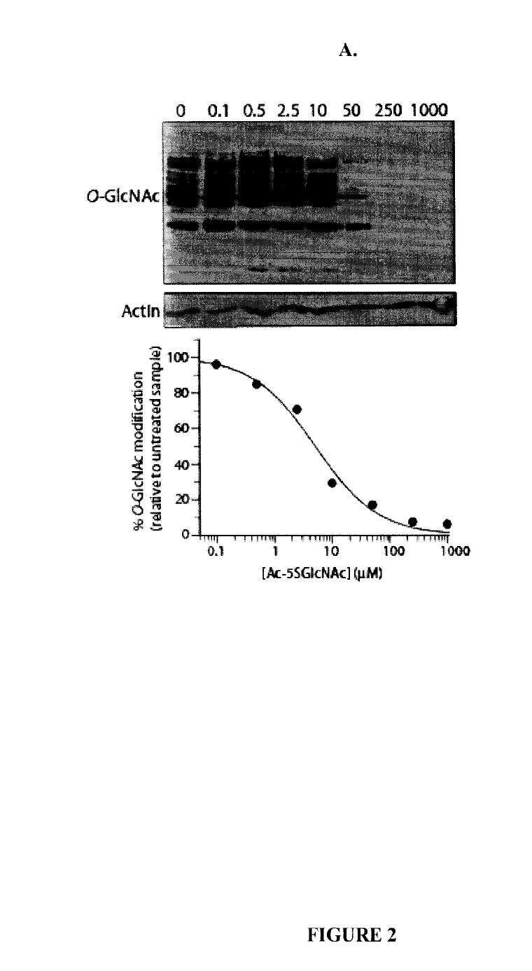

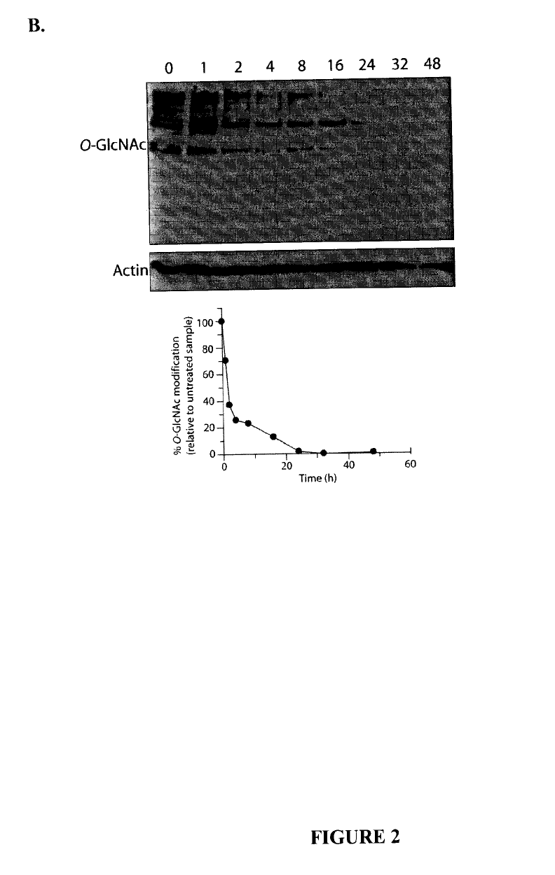

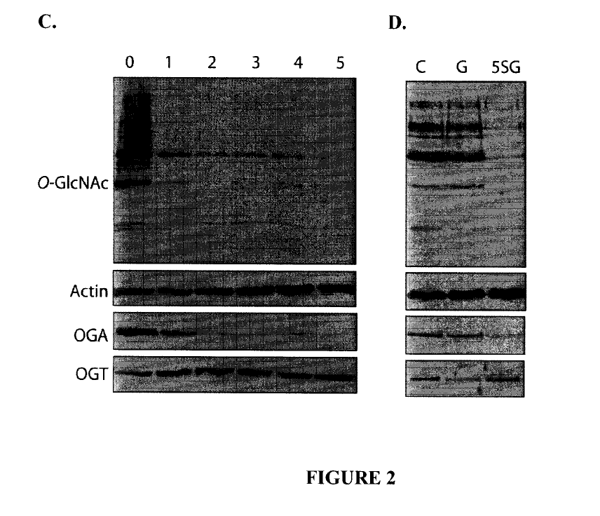

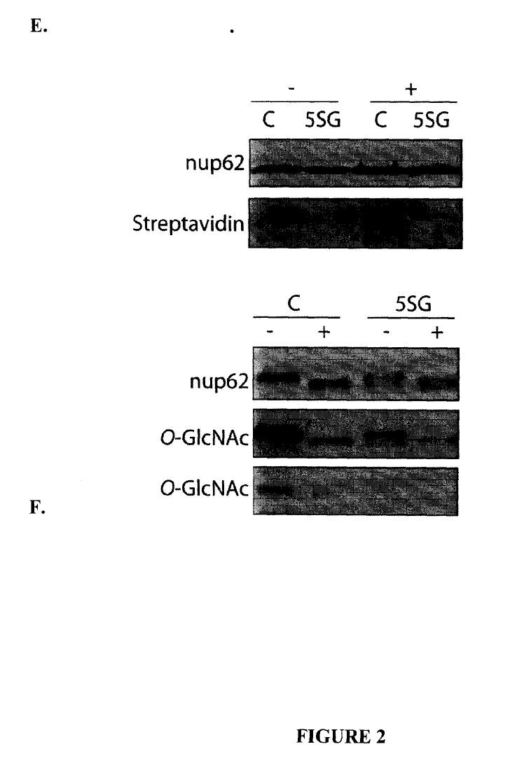

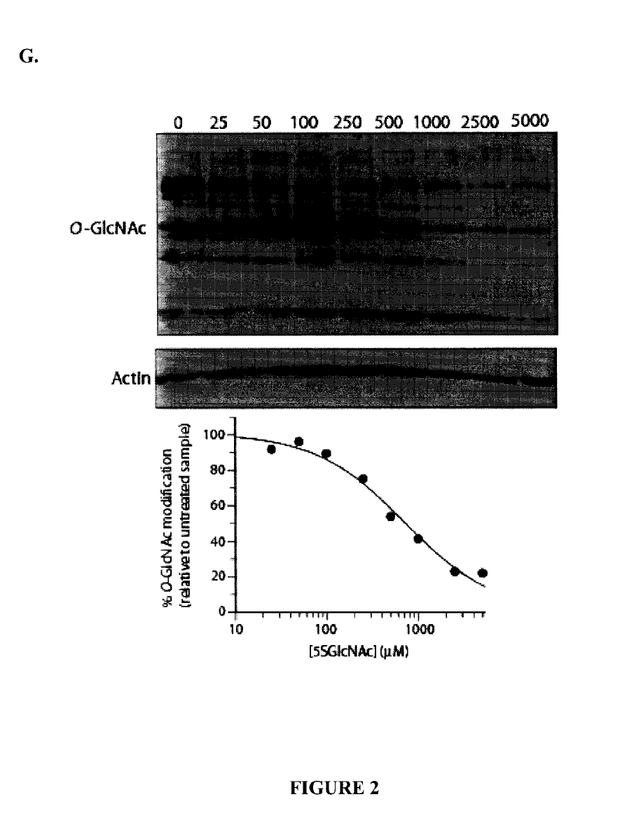

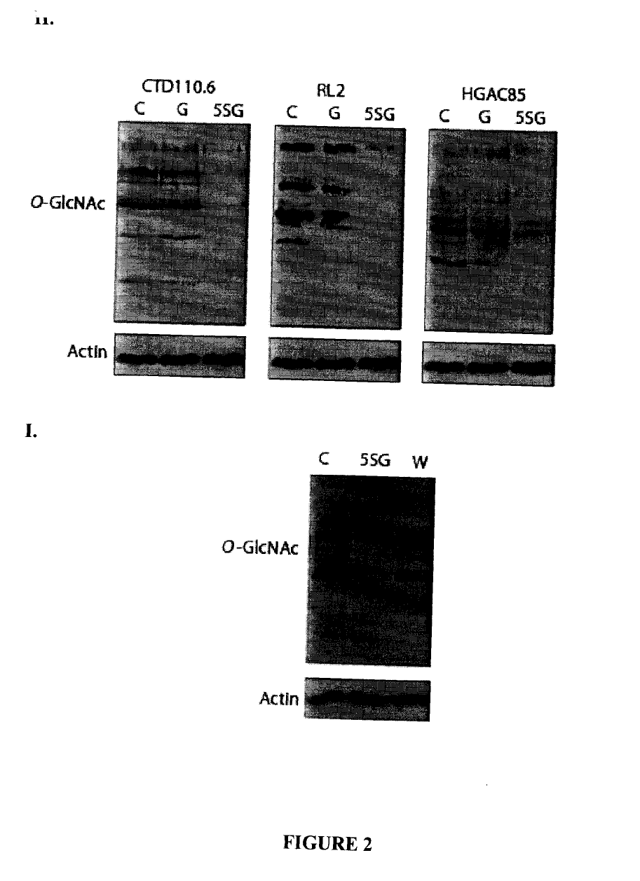

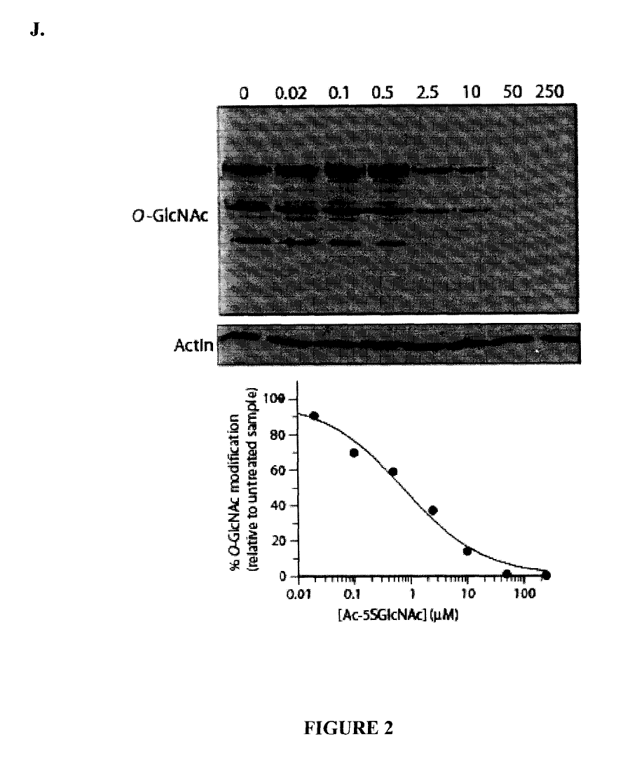

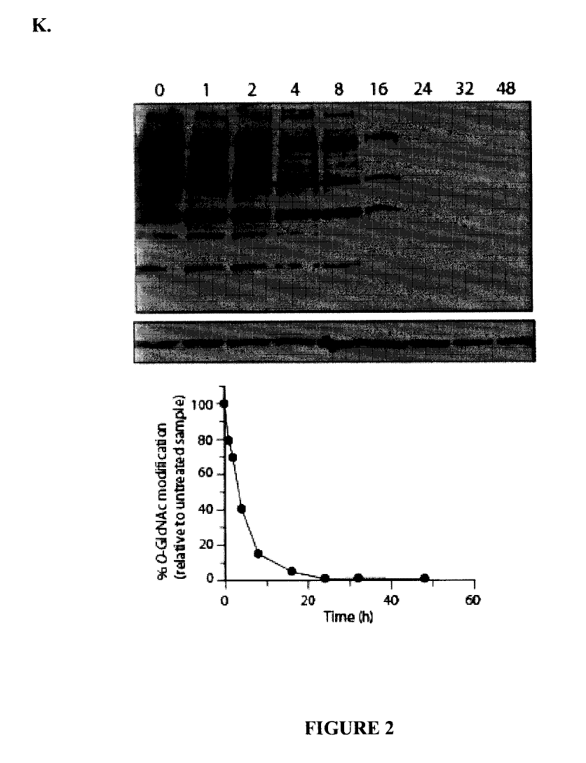

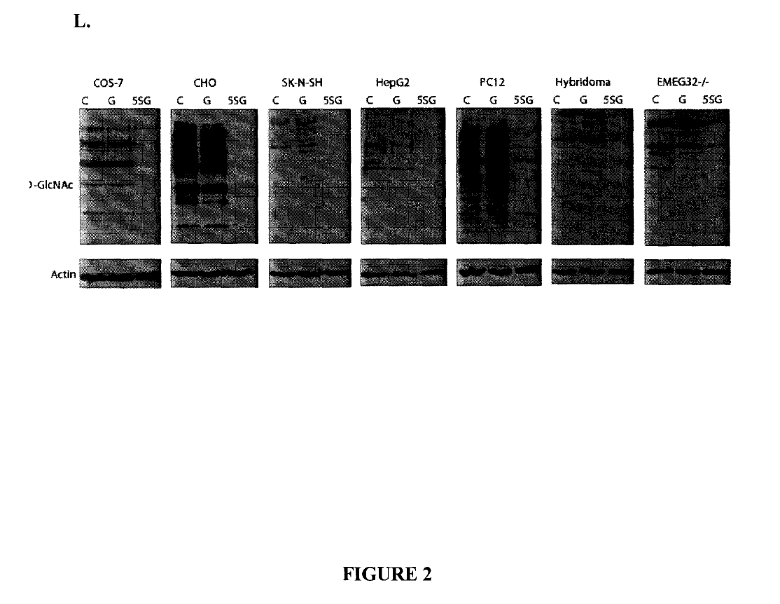

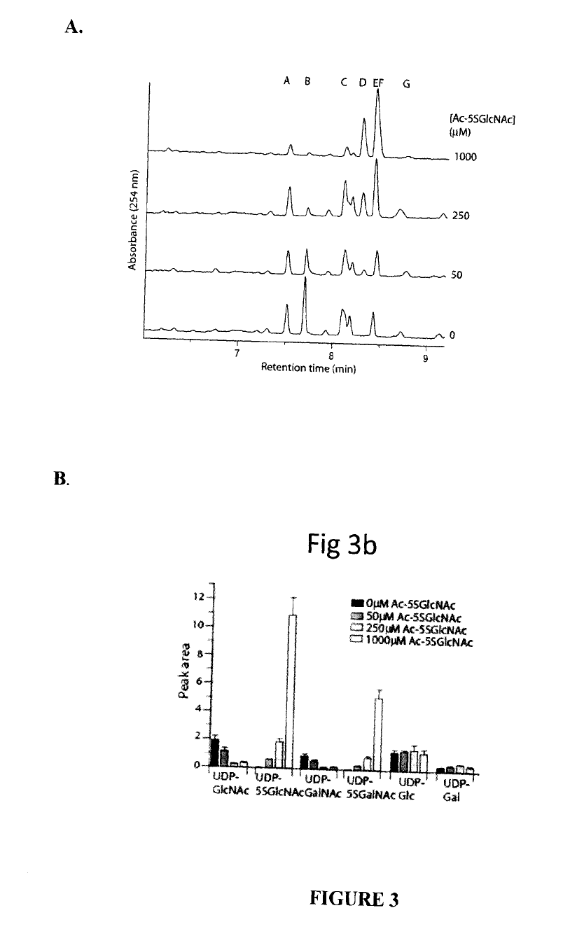

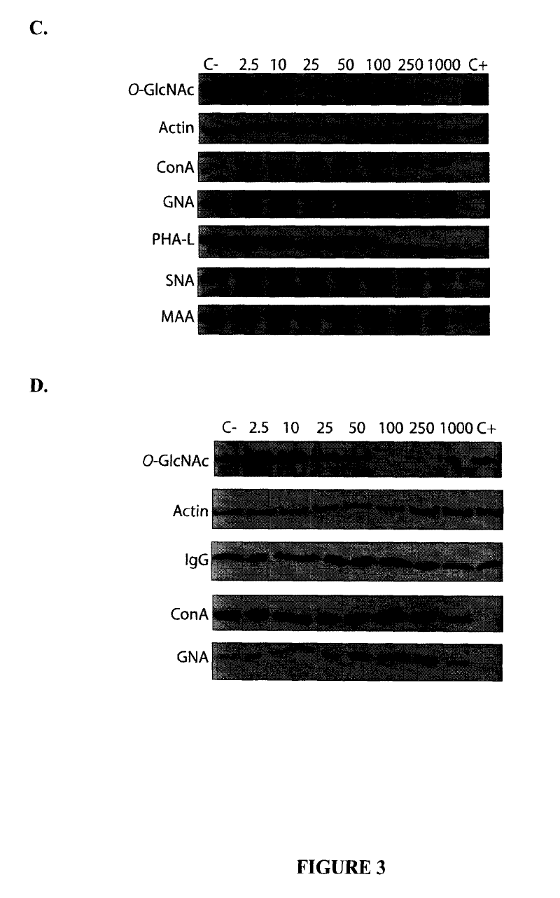

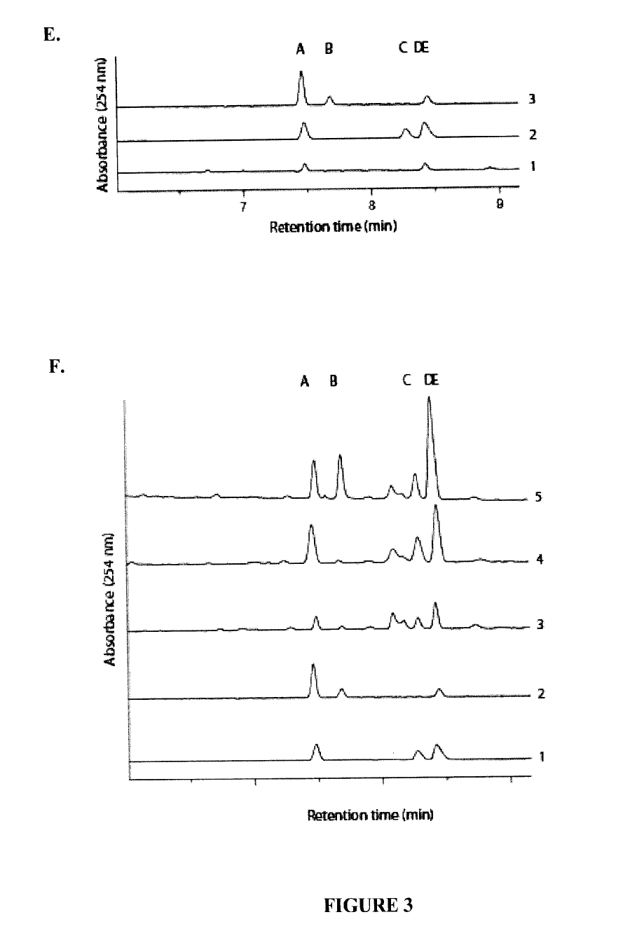

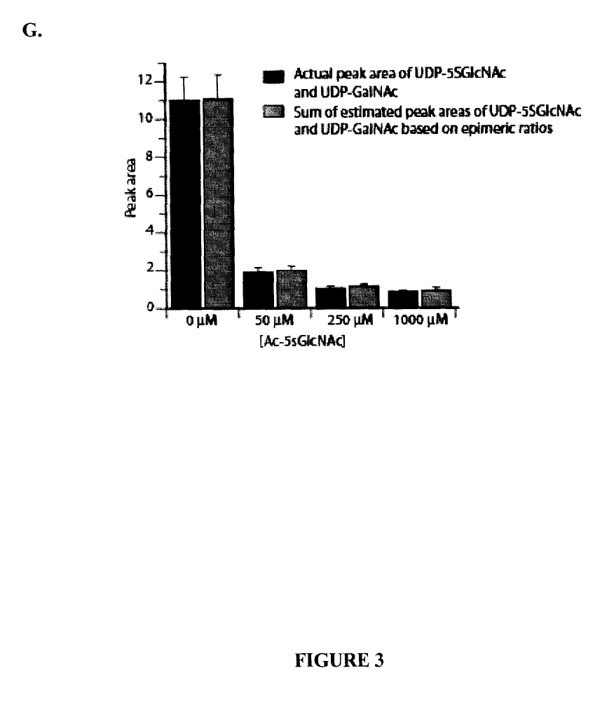

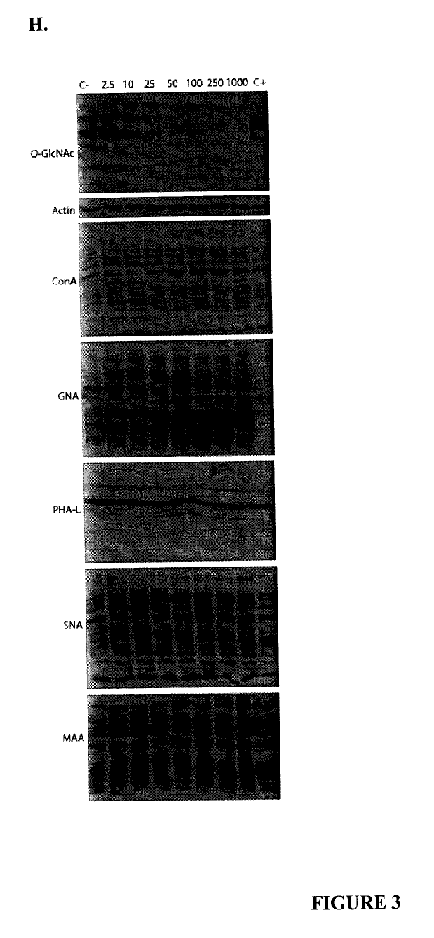

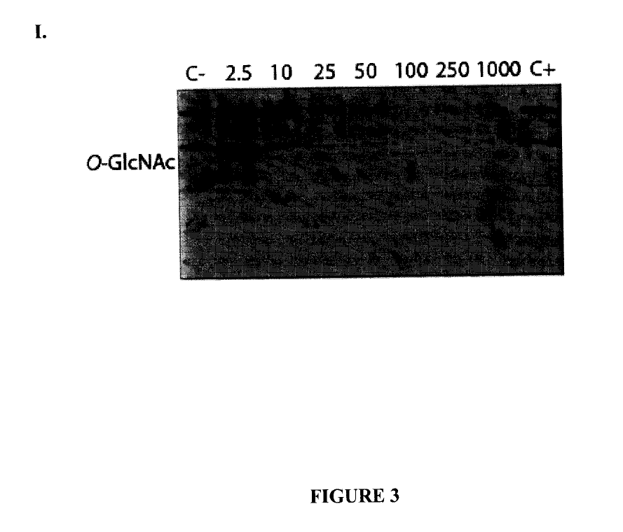

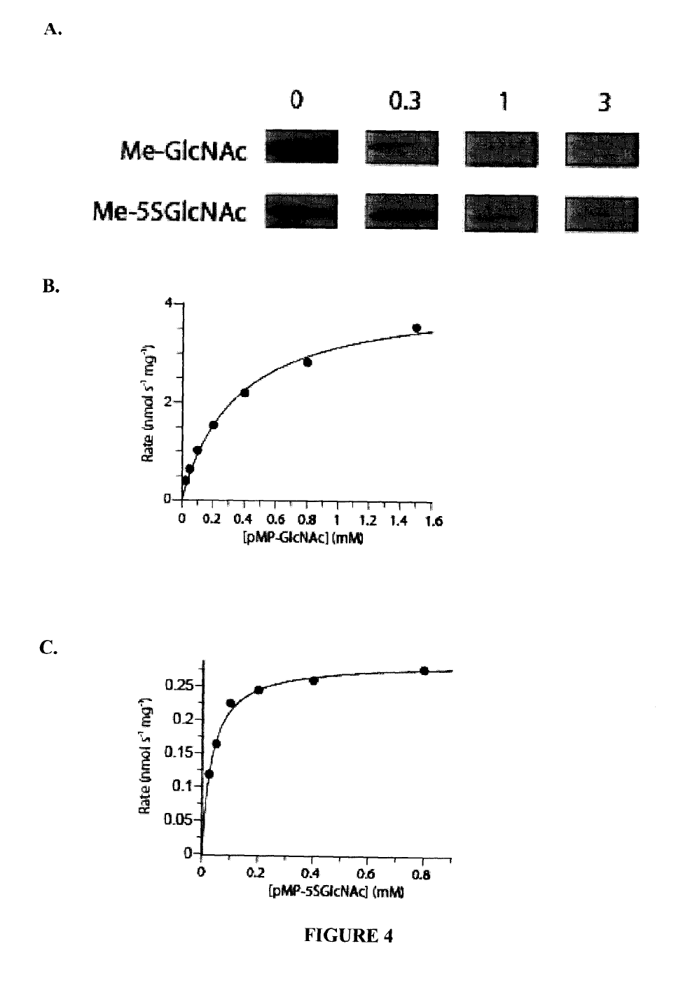

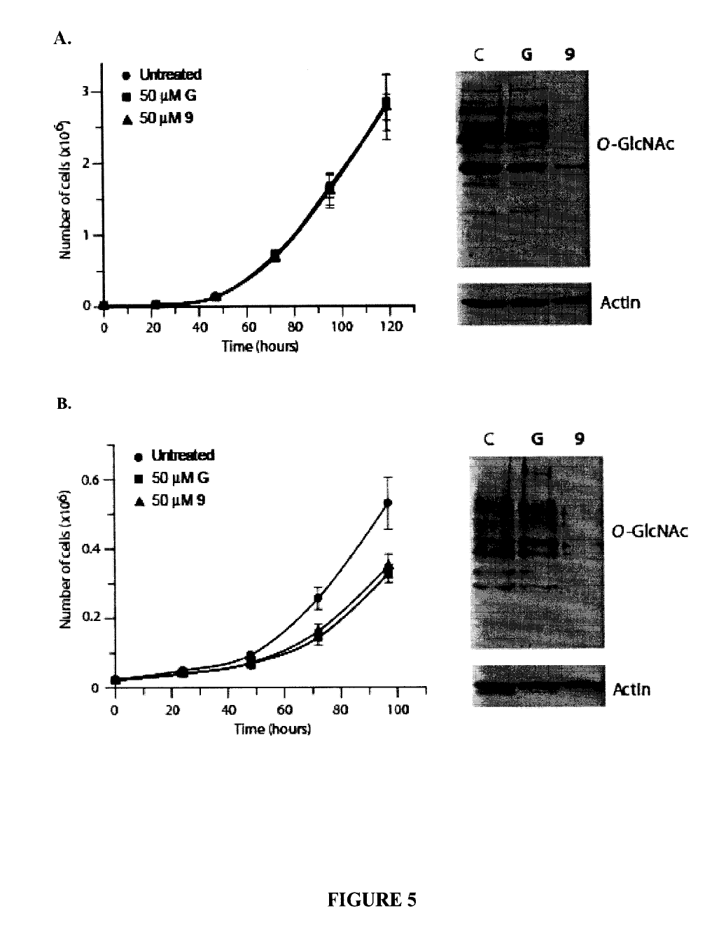

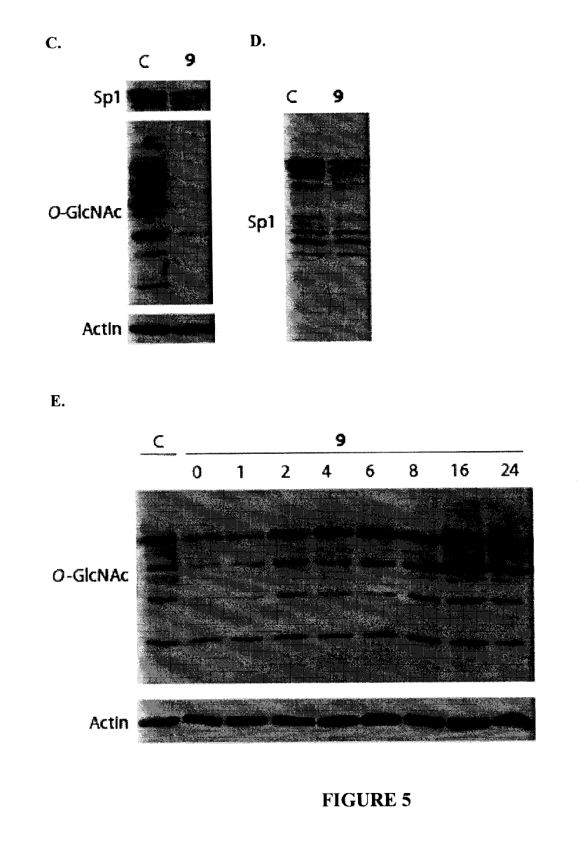

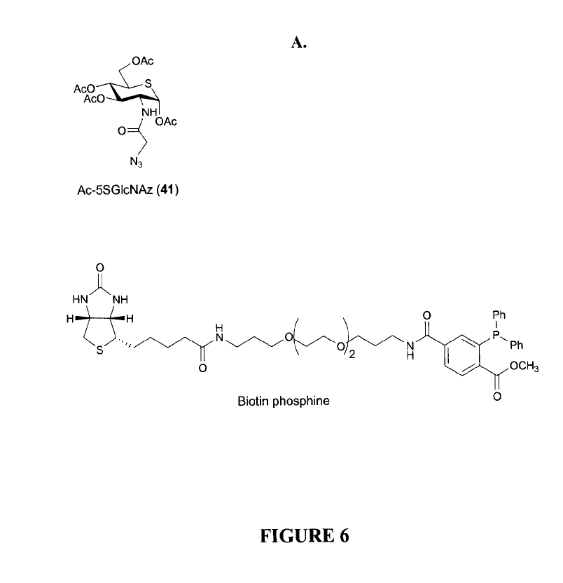

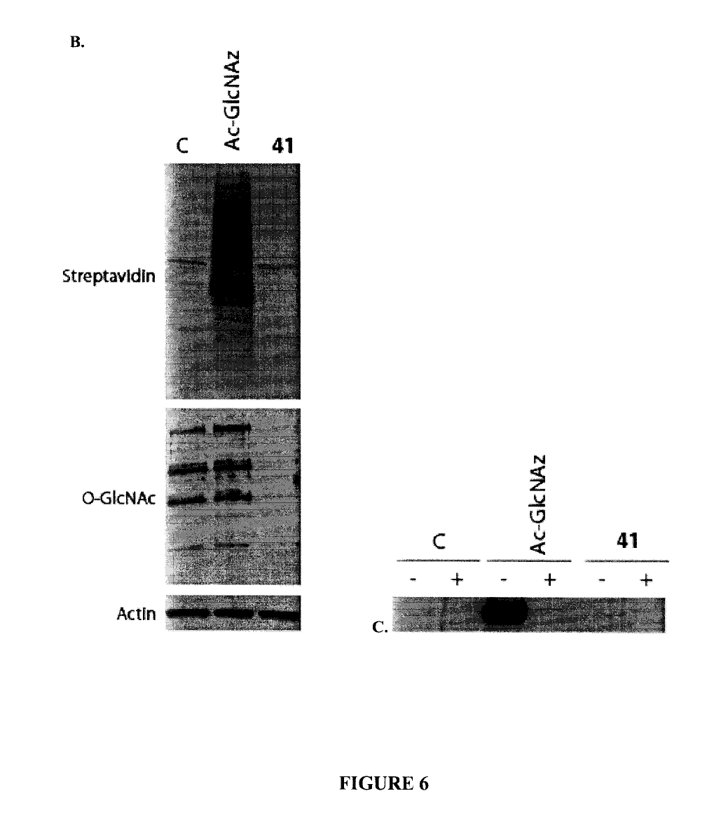

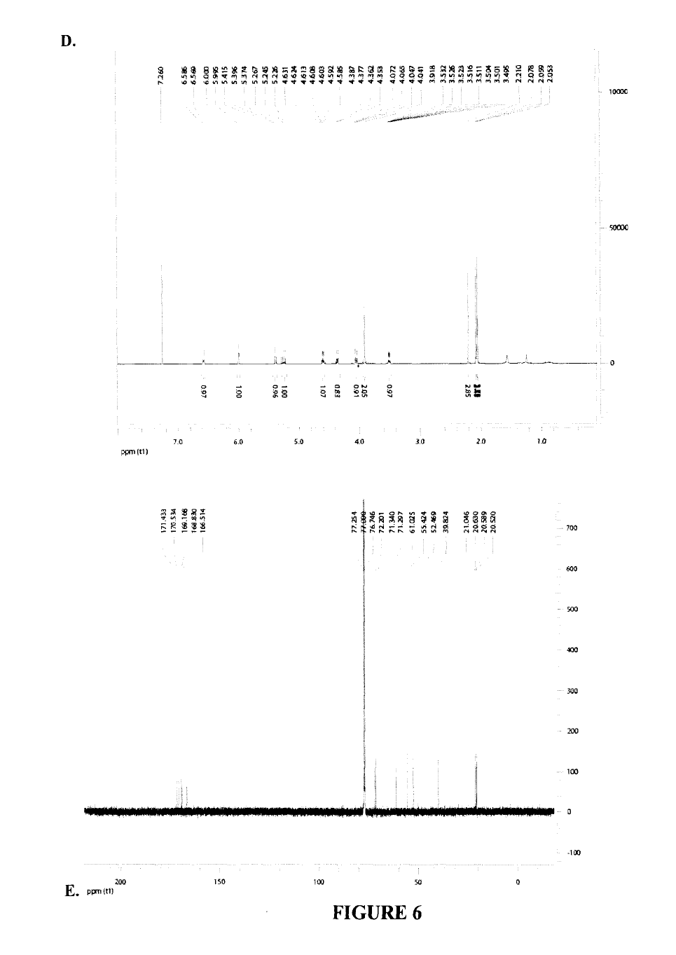

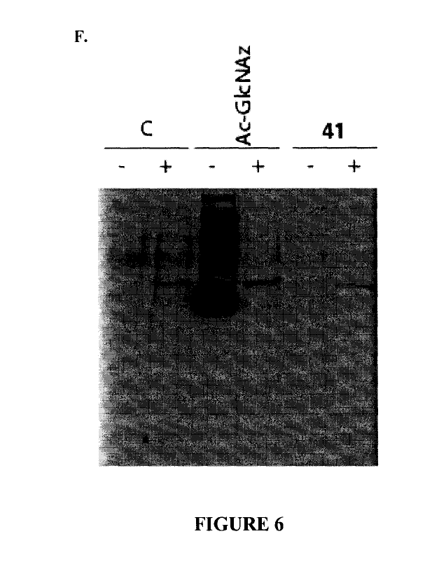

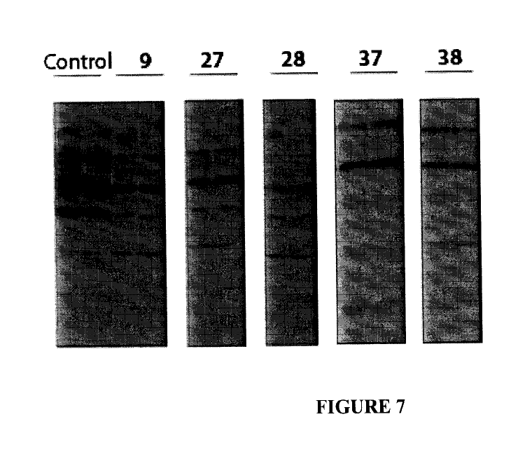

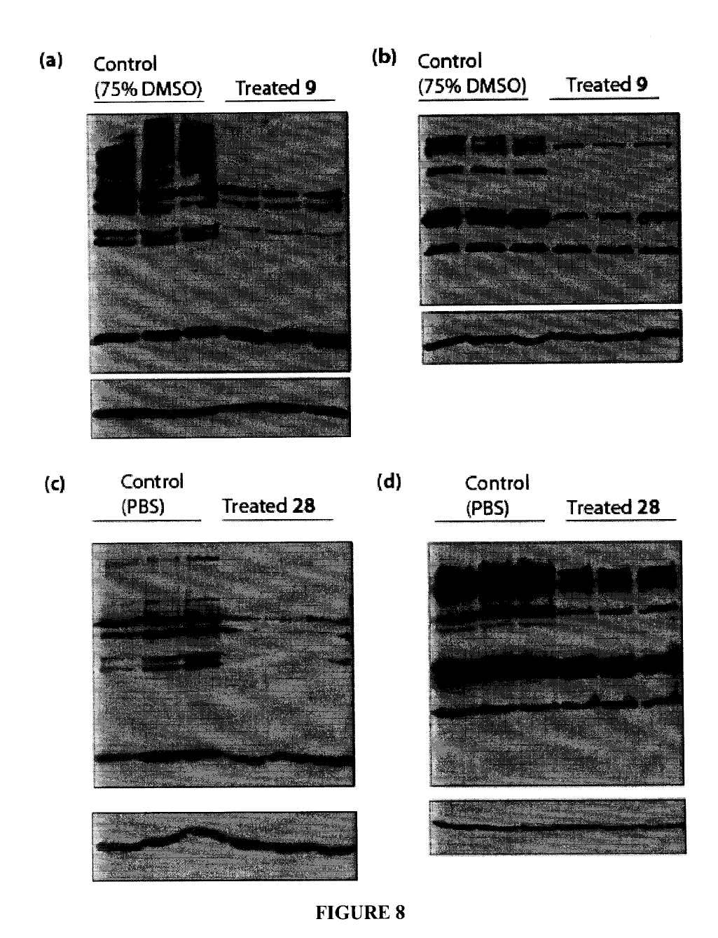

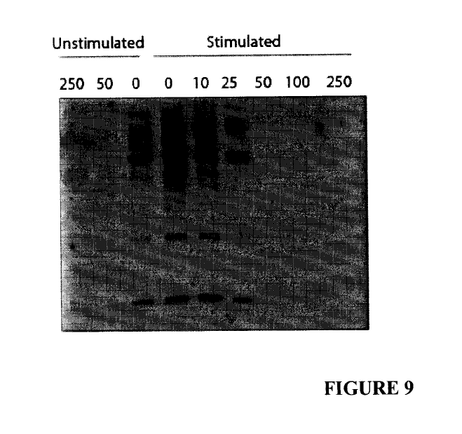

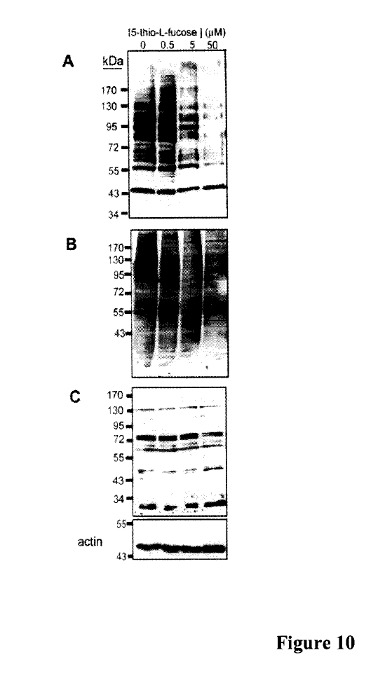

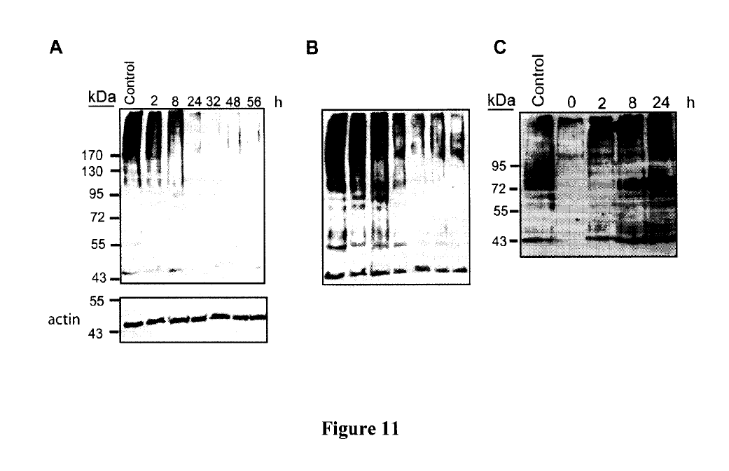

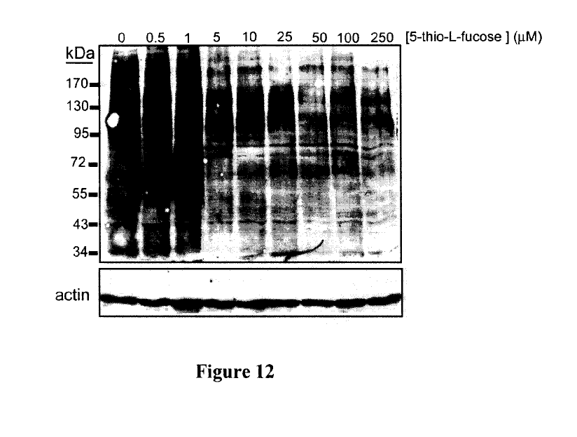

FIG. 2J. Western blots of CHO cell lysates following Ac-5SGlcNAc administration at different doses (0-250 μM) for 24 h. Upper panel, probed with anti-O-GlcNAc antibody (CTD110.6); lower panel, probed with anti-actin antibody. The plot shows the densitometry analysis converted to a % O-GlcNAc modification (corrected for actin levels) relative to the untreated control sample as a function of dose; this gives an EC₅₀ of 0.8 μM.

FIG. 2K. Western blots of CHO cell lysates following Ac-5SGlcNAc administration at 50 μM for different amounts of time (shown in hours). Upper panel, probed with anti-O-GlcNAc antibody (CTD110.6); lower panel, probed with anti-actin antibody. The plot shows the densitometry analysis converted to a % O-GlcNAc modification (corrected for actin levels) relative to the untreated control sample as a function of dose.

FIG. 2L. Western blots of cell lysates from different cell lines following no (C), Ac-GlcNAc (G) or Ac-5SGlcNAc (5SG) administration at 50 μM (or 100 μM for PC12 cells) for 24 h. Upper panel, probed with anti-O-GlcNAc antibody (CTD110.6); lower panel, anti-actin antibody. Cell lines used: COS-7 (African green monkey kidney cell line), CHO (Chinese hamster ovary cell line), SK-N-SH (human neuroblastoma cell line), HepG2 (human liver carcinoma cell line), PC12 (rat adrenal medulla pheochromocytoma cell line, which terminally differentiate upon nerve growth factor treatment), mouse hybridoma cell line and EMEG32^{-/-} (mouse embryonic fibroblasts deficient in glucosamine-6-phosphate acetyltransferase).

FIGS. 3A-D show that 5SGlcNAc is converted in cells to generate intracellular UDP-5SGlcNAc, causing only small perturbations in UDP-sugar nucleotide pools, and leaving N-glycosylation unperturbed.

FIG. 3A shows analysis of UDP-sugar pools from COS-7 cells treated with different concentrations of Ac-5SGlcNAc (0-1000 μM from bottom to top) for 24 h; the CE trace shows absorbance at 254 nm as a function of retention time. (A) GDP-Glc (internal standard); (B) UDP-GlcNAc; (C) UDP-Glc; (D) UDP-5SGalNAc; (E) UDP-5SGlcNAc; (F) UDP-GalNAc; (G) UDP-Gal. UDP-5SGlcNAc and UDP-GalNAc co-elute, but the amount of each could be estimated using the epimeric ratios determined from the standards (FIG. 3G).

FIG. 3B shows a bar chart showing relative concentrations of UDP-GlcNAc, UDP-5SGlcNAc, UDP-Gal, UDP-5SGal, UDP-Glc and UDP-Gal following treatment with 0-1000 μM Ac-5SGlcNAc.

FIG. 3C shows Western blots of COS-7 cell lysates following Ac-5SGlcNAc administration at different doses (0-1000 μM) for 24 h. C+ denotes untreated cell lysate incubated with PNGase F and C− denotes untreated cell lysate incubated with vehicle. Blots are probed with (from top to bottom) anti-O-GlcNAc antibody (CTD110.6), anti-actin antibody, ConA lectin (recognizes α-D-mannose, α-D-glucose and branched mannose), GNA lectin (recognizes mannose), PHA-L (recognizes complex branched chain oligosaccharide structure), SNA lectin (recognizes NeuAcα(2,6)Gal/GalNAc) and MAA lectin (recognizes NeuAcα(2,3)Gal). Full blots are shown in FIG. 3E.

FIG. 3D shows Western blots of mouse hybridoma cell lysates (O-GlcNAc and actin) and immunoprecipitated mouse hybridoma antibody (IgG, ConA and GNA) following administration of Ac-5SGlcNAc at different doses (0-1000 μM) for 24 h. C+ denotes untreated cell lysate incubated with PNGase F and C− denotes untreated cell lysate incubated with vehicle. Blots are probed with (from top to bottom) anti-O-GlcNAc antibody (CTD110.6) (full blot shown in FIG. 3F), anti-actin antibody, anti-IgG antibody, ConA lectin and GNA lectin.

FIG. 3E shows the monitoring UDP-5SGlcNAc and UDP-5SGalNAc in vitro by capillary electrophoresis; trace shows absorbance at 254 nm as a function of retention time. Run 1, UDP-5SGlcNAc; run 2, UDP-5SGlcNAc treatment with UDP-GlcNAc 4-epimerase; run 3, UDP-GlcNAc and UDP-GalNAc standards. (A) GDP-Glc (internal standard); (B) UDP-GlcNAc; (C) UDP-5SGalNAc; (D) UDP-5SGlcNAc; (E) UDP-GalNAc.

FIG. 3F shows UDP-sugar analysis by CE to validate identity of peaks from cells; trace shows absorbance at 254 nm as a function of retention time. Run 1, UDP-5SGlcNAc and UDP-5SGalNAc standards; run 2, UDP-GlcNAc and UDP-GalNAc standards; run 3, UDP-sugars extracted from COS-7 cells following treatment with 250 μM Ac-5SGlcNAc for 24 h; run 4, sample from run 3 spiked with standards from run 1; run 5, sample from run 3 spiked with standards from run 2. (A) GDP-Glc (internal standard); (B) UDP-GlcNAc; (C) UDP-5SGalNAc; (D) UDP-5SGlcNAc; (E) UDP-GalNAc.

FIG. 3G shows UDP-5SGlcNAc and UDP-GalNAc co-eluted during CE analysis. The contribution from each molecule to the peak could be calculated based on the concentration of UDP-GlcNAc and UDP-5SGaENAc, and the epimeric ratio determined from the standards of 2.1:1 GlcNAc:GalNAc. This graph shows a comparison of the total peak area compared to the sum of the estimated peak areas from the epimeric ratios for each concentration of Ac-5SGlcNAc administered to cells.

FIG. 3H shows western blots of COS-7 cell lysates following Ac-5SGlcNAc administration at different doses (0-1000 μM) for 24 h. C−, untreated cell lysate not incubated with PNGase F, C+, untreated cell lysate incubated with PNGase F. Blots are probed with (from top to bottom) anti-O-GlcNAc antibody (CTD 110.6), anti-actin antibody, ConA lectin (recognizes α-D-mannose, α-D-glucose and branched mannose), GNA lectin (recognizes mannose), PHA-L (recognizes complex branched chain oligosaccharide structure), SNA lectin (recognizes sialic acid α(2,6)Gal/GalNAc) and MAA lectin (recognizes sialic acid α(2,3)Gal).

FIG. 3I shows western blots of a mouse hybridoma cell lysates following Ac-5SGlcNAc administration at different doses (0-1000 μM) for 24 h. C−, untreated cell lysate not incubated with PNGase F, C+, untreated cell lysate incubated with PNGase F. Blot is probed with anti-O-GlcNAc antibody (CTD110.6).

FIG. 4A shows western blots of recombinantly modified p62 protein, probed with the anti-O-GlcNAc antibody CTD110.6 in the presence of increasing concentrations (in mM) of either Me-GlcNAc or Me-5SGlcNAc.

FIG. 4B shows Michaelis-Menten kinetics for OGA hydrolysis ofpMP-GlcNAc performed at 37° C. The fit gives a k_{cat} of 14.3 nmol s⁻¹ mg⁻¹ and a K_{M} of 350 μM.

FIG. 4C shows Michaelis-Menten kinetics for OGA hydrolysis ofpMP-5SGlcNAc performed at 37° C. The fit gives a k_{cat} of 0.28 nmol s⁻¹ me and a K_{M} of 35 μM.

FIG. 5A-E shows the effect of Ac-5SGlcNAc (9) on cell growth and Spl levels and testing the reversibility of Ac-5SGlcNAc (9) treatment. (A) Cell proliferation curves over the course of 5 days for CHO cells following no (circles), 50 μM Ac-GlcNAc (squares) or 50 μM Ac-5SGlcNAc (triangles) treatment. The error bars indicate the deviation from the mean for triplicate measurements. The western blots for the last time point indicate O-GlcNAc levels are still significantly decreased in cells treated with Ac-5SGlcNAc. Upper panel, probed with anti-O-GlcNAc antibody (CTD110.6); lower panel, probed with anti-actin antibody. (B) Cell proliferation curves over the course of 5 days for EMEG heterozygous mouse embryonic fibroblast cells following no (circles), 50 μM Ac-GlcNAc (squares) or 50 μM Ac-5SGlcNAc (triangles) treatment. The error bars indicate the deviation from the mean for triplicate measurements. The western blots for the last time point indicate O-GlcNAc levels are still significantly decreased in cells treated with Ac-5SGlcNAc. Upper panel, probed with anti-O-GlcNAc antibody (CTD110.6); lower panel, probed with anti-actin antibody. (C) Western blots of CHO cell lysates following no (C) or Ac-5SGlcNAc administration at 50 μM for 4 days. Probed with (from top to bottom) anti-Spl antibody, anti O-GlcNAc (CTD110.6) antibody and anti-actin antibody. Full blot of the anti-Sp 1 is shown in panel (d). (D) Western blots of CHO cell lysates following no (C) or Ac-5SGlcNAc administration at 50 μM for 4 days. The full blot probed with anti-Spl antibody is shown. (E) Western blots of COS-7 cell lysates following no (C) or 50 μM Ac-5SGlcNAc treatment for 24 h. Following treatment, the media was replaced with no inhibitor, and cells were harvested at the time points indicated (between 0 and 24 h). Upper panel, probed with anti-O-GlcNAc antibody (CTD110.6); lower panel, probed with anti-actin antibody.

FIG. 6A-E shows metabolic feeding of Ac-5SGlcNAz (41) to cells causes a decrease in O-GlcNAc levels, but chemoselecctive ligation demonstrates there is no accumulation of 5SGlcNAz on proteins. (A) Structures of the molecules used or generated during the chemoselective ligation study (X═O or S as indicated, R═H or Ac as indicated). (B) Western blots of COS-7 cell lysates administered specified agents at 50 μM for 24 h; vehicle only (C), Ac-GlcNAz or Ac-5SGlcNAz (41). Cells were harvested and then underwent the Staudinger ligation with biotin phosphine. Blots are probed with (from top to bottom) streptavidin-HRP, anti O-GlcNAc (CTD110.6) antibody and anti-actin antibody. (C) Western blot of immunoprecipitated nup62 from cell lysates following vehicle (C), 50 μM Ac-GlcNAz or Ac-5SGlcNAz (41) treatment for 24 h. Following immunoprecipitation, nup62 was incubated with buffer (−) or with BtGH84 (+) for 2 h to remove O-GlcNAc, and then underwent the Staudinger ligation with biotin phosphine. Blots are probed with streptavidin-HRP. (D) ¹H-NMR spectrum of Ac-5SGlcNAz (41). (E) ¹³C-NMR spectrum of Ac-5SGlcNAz (41). Both were taken in CDCl₃. (F) Full western blot of immunoprecipitated nup62 from cell lysates following vehicle (C), 50 μM Ac-GlcNAz or Ac-5SGlcNAz (41) treatment for 24 h. Following immunoprecipitation, nup62 was incubated with buffer (−) or with BtGH84 (+) for 2 h to remove O-GlcNAc, and then underwent the Staudinger ligation with biotin phosphine. Blots are probed with streptavidin-HRP.

FIG. 7 shows western blots probed with anti-O-GlcNAc antibody (CTD110.6) following treatment of COS-7 cells with 50 μM compound 9, 27, 28, 37 or 36 or vehicle alone for 24 h.

FIG. 8 shows representative western blots from mouse studies. Either compound 9 or 28 (300 mg/kg intraperitoneally) or vehicle (75% DMSO or PBS) was dosed in triplicate for 16 hours, and subsequently tissues harvested. (a) Lung tissue following treatment with compound 9; (b) Pancreatic tissue following treatment with compound 9; (c) Lung tissue following treatment with compound 28; (d) Pancreatic tissue following treatment with compound 28. In all cases the top panel the blot is probed with anti-O-GlcNAc antibody (CTD 110.6) and bottom panel with anti-actin antibody.

FIG. 9 shows western blot, probed with anti-O-GlcNAc antibody (CTD110.6) of primary CLL cells treated with a range of Ac-5SGlcNAc (9) concentrations (shown in μM) in the presence or absence of cytokine stimulation.

FIG. 10 shows 5-thiofucose (5-T-Fuc) reduces sialyl-lewis X expression in HepG2 liver cells. Immunoblot analysis of HepG2 lysates was performed with (A) anti-CD15s (sLe^{X}), (B) Aleura aurantia lectin (binds fucosylated glycans) and (C) the α2,6-Neu5Ac-specific Maackia amurensis lectin.

FIG. 11 shows 5-T-Fuc reduces sLeX expression in a time-dependant fashion. Immunoblot analysis of sLeX expression in HepG2 cells cultured for an increasing length of time in the presence of 5-T-Fuc. (A) 0.5 min and (B) 3 min exposures. (C) Full recovery of sLeX expression after a 24 h exposure to 5-T-Fuc occurs within 24 h.

FIG. 12 shows CHO K1 cells demonstrate a reduction in core (α1,6)-fucosylation upon 5-T-Fuc-treatment.

### DETAILED DESCRIPTION

The invention provides, in part, compounds that are capable of inhibiting a glycosyltransferase or “GT”. In some embodiments, the GT may be a uridine diphospho-N-acetylglucosamine:polypeptide(3-N-acetylglucosaminyltransferase or “OGT”). In alternative embodiments, the GT may be an alternative N-acetylglucosaminyltransferase, an N-acetylgalactosaminyltransferase, a fucosyltransferase, a xylotransferase or a sialyltransferase.

In some embodiments, the invention provides for the design of an inhibitor of a GT (a “GT inhibitor”) by providing a nucleotide sugar substrate precursor or variant or analog thereof that is readily cell permeable and is capable of being utilized in an endogenous biosynthetic pathway to form the GT inhibitor.

In some embodiments, a GT inhibitor may be constructed by replacing the endocyclic oxygen atom found in sugars to a different group including, without limitation, sulphur, selenium, or CH₂, such that enzymes in biosynthetic pathways involved in the formation of a nucleotide sugar still process the sugar analogue to form, within tissues, an analogue of the natural nucleotide sugar in which the endocyclic ring oxygen is replaced by the group present in the sugar analogue precursor. Accordingly, by using sugar analogues in which the endocyclic oxygen is replaced by certain groups, the sugar analogue is assimilated by these salvage and biosynthetic pathways to form the unnatural nucleotide sugar analogue having the group present in place of the endocyclic sugar ring oxygen.

In some embodiments, the resulting nucleotide sugar analogue is either not a substrate or it is a worse substrate for the GT than is the naturally occurring nucleotide sugar used by the GT. In some embodiments, the unnatural nucleotide sugar analogue binds to and thereby inhibits the GT. In some embodiments, the activity of the GT is impaired and the levels of the glycoconjugates normally biosynthesized by the GT decrease. The levels of the GT may, in some cases, increase to compensate for its inhibition.

In the exemplary embodiment of OGT inhibition, as described herein, we show that the change from oxygen to sulphur was subtle enough for the enzymes in the salvage and biosynthetic pathways to tolerate the synthesis of the nucleotide sugar analogue, but sufficient to impair the ability of OGT to use the nucleotide sugar analogue and cause inhibition of OGT.

Other sugars having the endocyclic oxygen or other group replaced by a different group, as described herein, will also feed into the normal biosynthetic pathways of the cell to form nucleotide sugar analogues. These nucleotide sugar analogues are poor substrates for GTs that process their preferred natural nucleotide sugar substrates. Without being bound to any particular hypothesis, it is expected that because the nucleotide sugar analogues are turned over poorly by GTs, yet resemble the natural nucleotide sugar substrates used by these GTs, these nucleotide sugar analogues inhibit the normal functioning of these GTs both in vitro and in tissues and in vivo.

In alternative embodiments, unnatural sugar-l-phosphate analogues in which the endocyclic ring oxygen is replaced for example by CH₂, S, or Se, or which have other S or Se substitutions as described herein, may be used in the biosynthesis within cells of the corresponding unnatural nucleotide sugar which would, as discussed herein, inhibit GTs processing the corresponding natural nucleotide sugar phosphate. Several examples are provided herein, for example, formulae VI-X, wherein the sugar-l-phosphate is derivatized. Certain compounds described by these formulae are assimilated by intracellular biosynthetic pathways to form the unnatural nucleotide sugar that will inhibit the corresponding GT(s). Accordingly, a number of sugars, including GlcNAc, GalNAc, ManNAc, Fuc, Xyl, or GlcNH2, as well as their 1-phosphosugar derivatives, may be used as described herein to construct GT inhibitors.

A “glycosyltransferase” or “GT” is an enzyme that transfers a sugar residue from an anionic nucleotide sugar donor to various acceptor molecules, such as proteins, lipids, saccharides, metabolites, or other substrates. GTs include, without limitation, N-acetylglucosaminyltransferases, N-acetylgalactosaminyltransferases, fucosyltransferases, xylosyltransferases, sialyltransferases, mannosyltransferases, glucosyltransferases and galactosyltransferases.

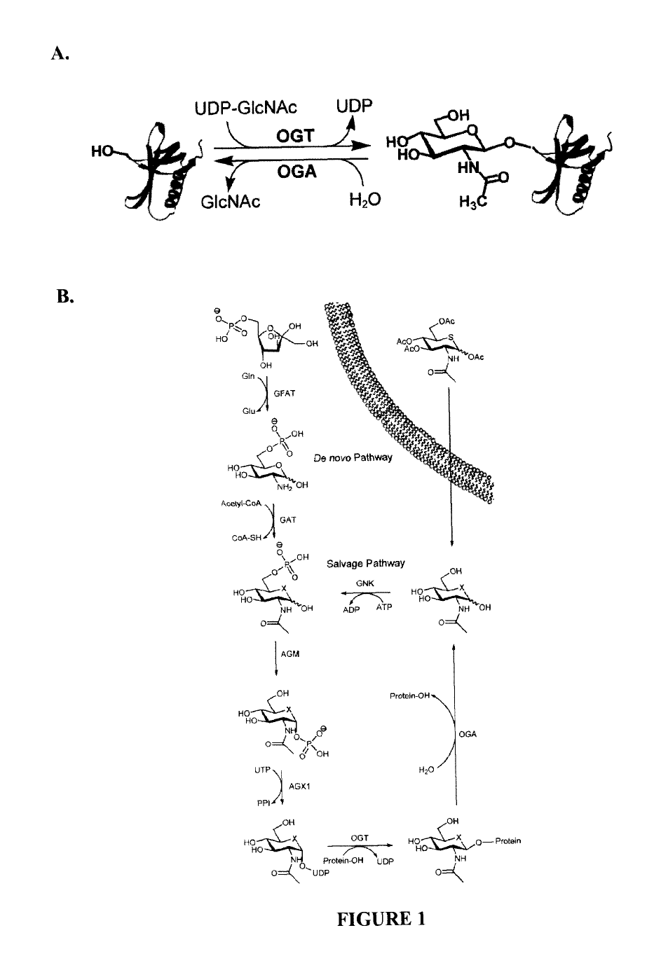

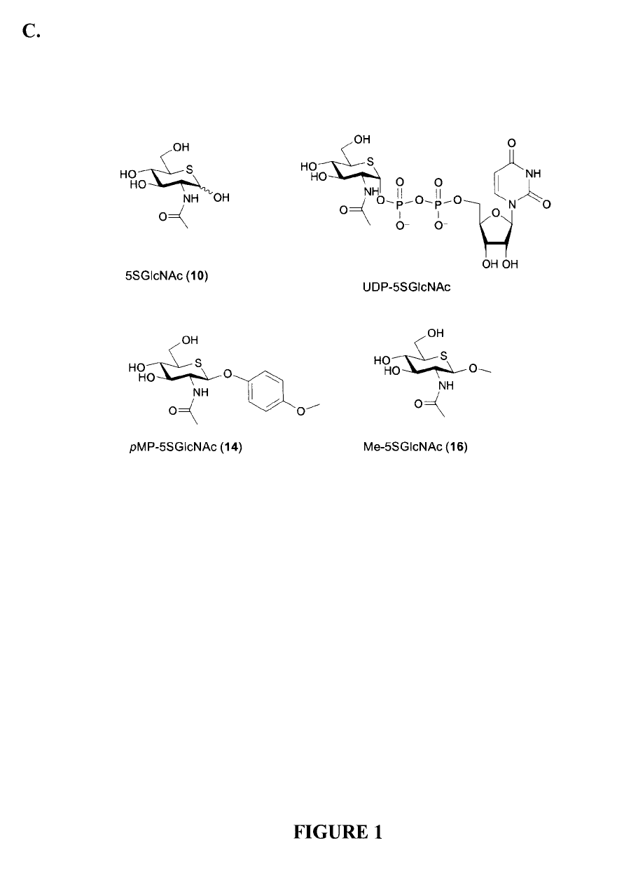

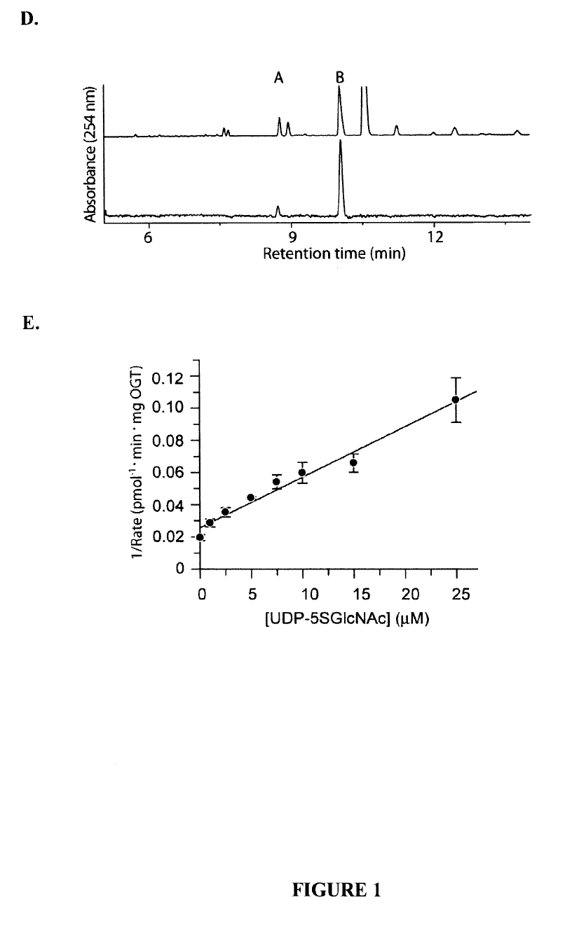

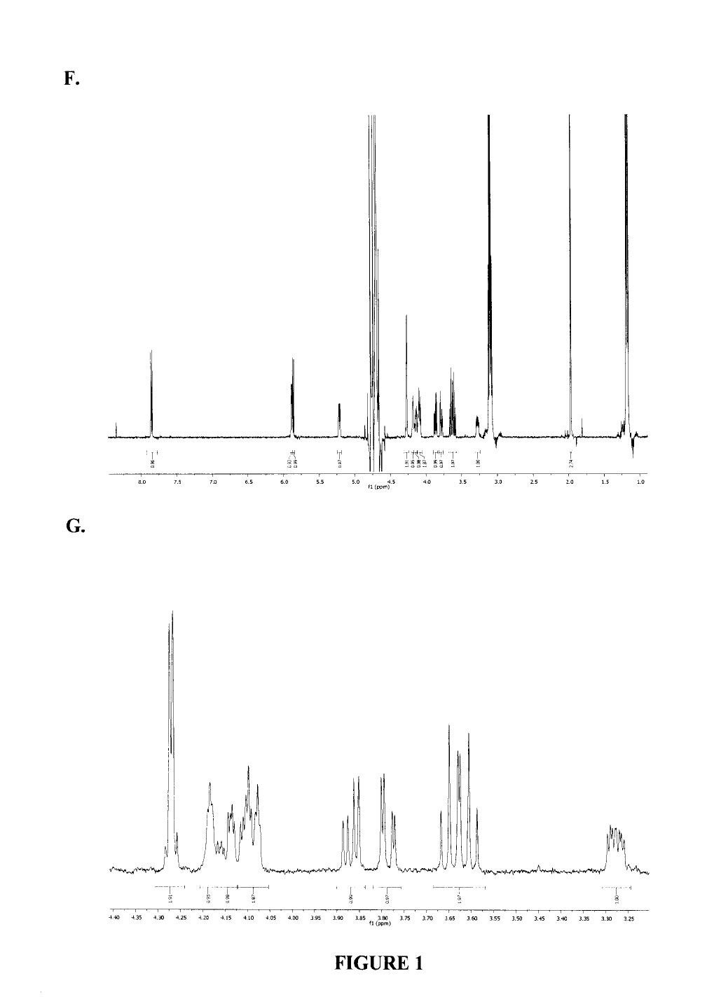

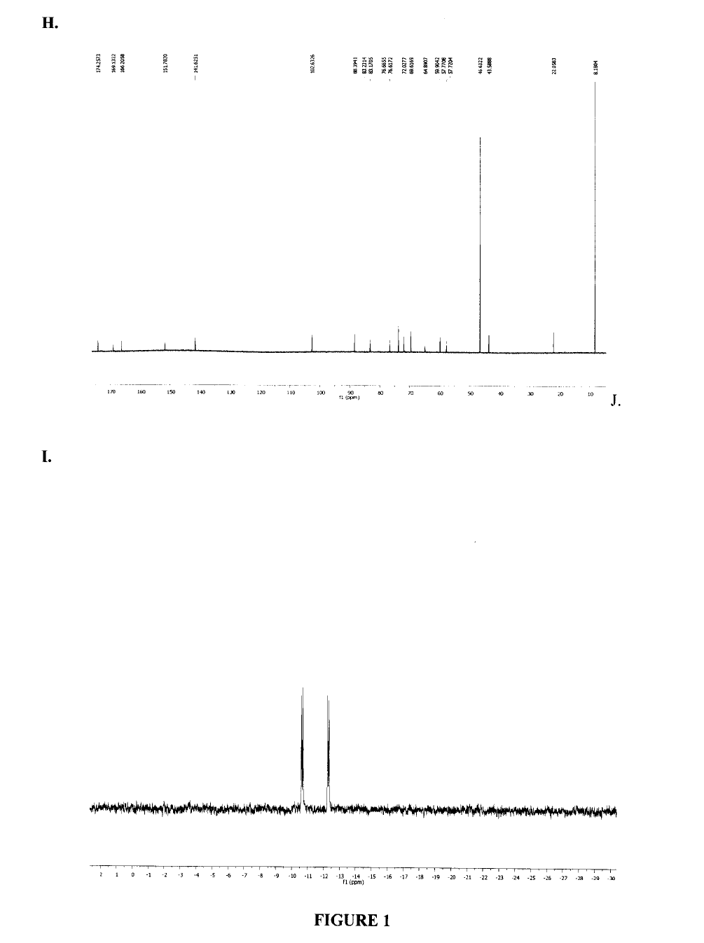

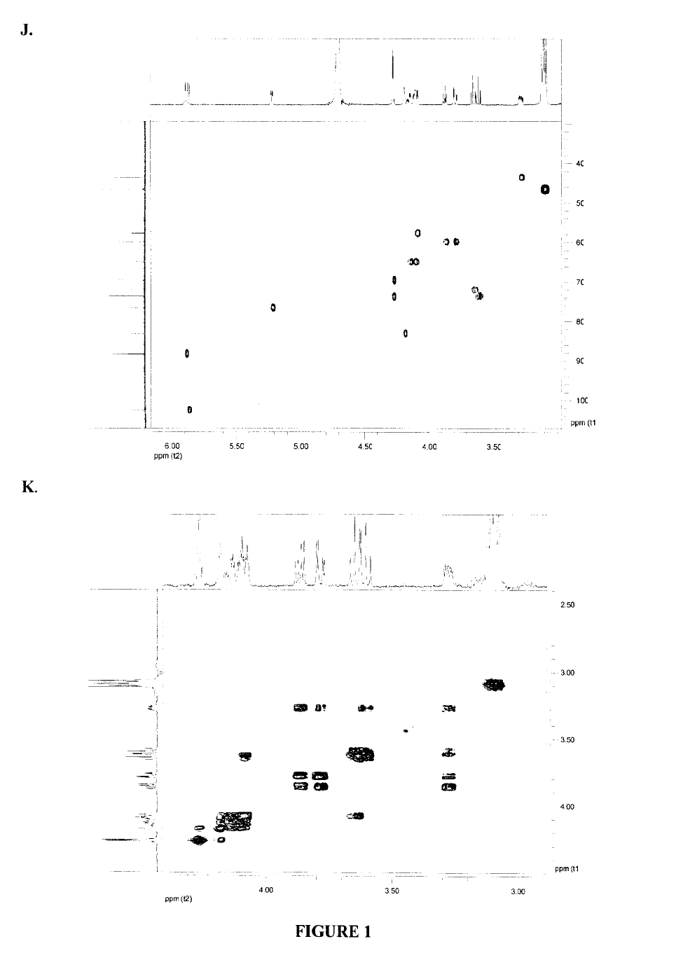

A “uridine diphospho-N-acetylglucosamine:polypeptide β-N-acetylglucosaminyltransferase” or “OGT” is a GT that transfers O-GlcNAc onto proteins or polypeptides using the donor substrate uridine diphospho-N-acetylglucosamine (UDP-GlcNAc), which is biosynthesized through the action of enzymes comprising the hexosamine biosynthetic pathway (HBP) (FIG. 1B). As used herein, an OGT may be derived from any source or subject or in different splice forms. Examples of OGTs include, without limitation human OGT having Accession number O15294, as well as OGT splice variants, for example those having Accession numbers NP_{—}858058.1 or CAC86128.1 (isoform 1), NP_{—}858059.1 or CAC86127.1 (isoform 2), or CAC86129 (mitochondrial variant). In alternative embodiments, OGTs include OGTs from various organisms, for example, rat, mouse, human, monkey, etc. In alternative embodiments, an OGT as used herein is substantially identical to the human OGT having Accession number O15294, or to OGT splice variants, for example those having Accession numbers NP_{—}858058.1 or CAC86128.1 (isoform 1), NP_{—}858059.1 or CAC86127.1 (isoform 2), or CAC86129 (mitochondrial variant), or to homologous sequences found in for example rat, mouse, monkey, etc.

A “fucosyltransferase” or “FUT” is a GT that transfers fucose onto proteins, polypeptides, or other saccharide units, using the donor substrate guanosine diphospho-fucose (GDP-Fuc), which is biosynthesized through the action of enzymes comprising the fucose biosynthetic pathway or salvaged via the fucose salvage pathway⁷⁴. There are currently thirteen known fucosyltransferase genes known within the human genome⁶⁷.

Some of these are involved in the synthesis of Lewis blood group antigens including sialyl Lewis X (sLe^{x}) while others synthesize Le^{a}, Le^{b}, sialyl Le^{a67}. Some of these fucosyltransferases have other activities or the activities remain to be discovered. There are also two protein O-fucosytransferases that are known as Pofut1 and Pofut2, and these transfer a fucose residue to serine or threonine within epidermal growth factor-like repeats or thrombospondin type 1 repeats to form an α-linkage⁶⁷. As used herein, a fucosyltransferase may be derived from any source or subject or in different splice forms. Examples of FUTs include, without limitation human FUT1 through FUT11 and includes poFUT1 and poFUT2 as well as FUT splice variants. In alternative embodiments, FUTs include FUTs from various organisms, for example, rat, mouse, human, monkey, etc.

A “biomolecule” refers to a molecule or compound produced by a living organism. Biomolecules include without limitation proteins, peptides, nucleics acids (e.g., DNA or RNA), polysaccharides, lipids, small molecules, etc. In some embodiments, a biomolecule as used herein may be modified with a nucleotide sugar molecule.

By “substantially identical” is meant an amino acid or nucleotide sequence that differs from a reference sequence only by one or more conservative substitutions, as discussed herein, or by one or more non-conservative substitutions, deletions, or insertions located at positions of the sequence that do not destroy the biological function of the amino acid or nucleic acid molecule. Such a sequence can be any integer from 10% to 99%, or more generally at least 10%, 20%, 30%, 40%, 50%, 55% or 60%, or at least 65%, 75%, 80%, 85%, 90%, or 95%, or as much as 96%, 97%, 98%, or 99% identical when optimally aligned at the amino acid or nucleotide level to the sequence used for comparison using, for example, the Align Program⁷⁵ or FASTA. For polypeptides, the length of comparison sequences may be at least 2, 5, 10, or 15 amino acids, or at least 20, 25, or 30 amino acids. In alternate embodiments, the length of comparison sequences may be at least 35, 40, or 50 amino acids, or over 60, 80, or 100 amino acids. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various substitutions, deletions, substitutions, and other modifications.

Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8× SSC, 0.2 M Tris-Cl, pH 7.6, 1× Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al.⁷⁶.

By “inhibits,” “inhibition” or “inhibiting” means a decrease by any value between 10% and 90%, or of any integer value between 30% and 60%, or over 100%, or a decrease by 1-fold, 2-fold, 5-fold, 10-fold or more. It is to be understood that the inhibiting does not require full inhibition. In some embodiments, an inhibitor of a GT reduces the levels of glycoconjugates biosynthesized by the GT. In some embodiments, an inhibitor of an OGT decreases or reduces O-GlcNAc levels e.g., O-GlcNAc-modified polypeptide or protein levels, in cells, tissues, or organs (e.g., in pancreatic, brain, muscle, adipose, hepatic, blood, skin, eye, nervous system tissue) and in animals. By “reduced,” “reduces” or “reducing” is meant a decrease by any value between 10% and 90%, or of any integer value between 30% and 60%, or over 100%, or a decrease by 1-fold, 2-fold, 5-fold, 10-fold, 15-fold, 25-fold, 50-fold, 100-fold or more.

In some embodiments, the compounds of the present invention according to Formulae I or VI reduce O-GlcNAc levels on O-GlcNAc-modified polypeptides or proteins in vivo specifically via interaction with an OGT, and are effective in treating conditions which require or respond to inhibition of OGT activity.

In some embodiments, the compounds of the present invention are useful as agents that produce a decrease in the levels of specific sets of glycoconjugates. In some embodiments, the compounds such as those described in Formulae II-V or VII-X are therefore useful to treat conditions which require or respond to inhibition of GT activity, such as N-acetylglucosaminyltransferases, N-acetylgalactosaminyltransferases, fucosyltransferases, xylosyltransferases, sialyltransferases, mannosyltransferases, glucosyltransferases or galactosyltransferases.

In some embodiments, the compounds of the present invention according to Formulae IV or IX reduce levels of fucose modified biomolecules in vivo via interaction with an FUT, and/or through decreasing GDP-Fuc levels in cells, and are effective in treating conditions which require or respond to decreased FUT activity.

In some embodiments, the invention provides methods of producing an antibody by contacting an antibody-producing cell with a fucosyltransferase inhibitor, such that an antibody with reduced levels of fucose is produced. By “reduced levels of fucose” is meant that the glycan structures present on the antibody contain decreased amounts of fucose. By “reduced,” “reduces” or “reducing” is meant a decrease by any value between 10% and 90%, or of any integer value between 30% and 60%, or over 100%, or a decrease by 1-fold, 2-fold, 5-fold, 10-fold, 15-fold, 25-fold, 50-fold, 100-fold or more.

In some embodiments, the compounds produce a decrease in levels of O-GlcNAc modification on O-GlcNAc-modified polypeptides or proteins, and are therefore useful for treatment of disorders responsive to such decreases in O-GlcNAc modification; these disorders include without limitation cancer, diabetes, insulin resistance, complications of diabetes, inflammation, autoimmune disease or bacterial infections. In some embodiments, the compounds are also useful as a result of other biological activities related to their ability to inhibit the activity of glycosyltransferases. In alternative embodiments, the compounds of the invention are valuable tools in studying the physiological role of O-GlcNAc at the cellular and organismal level.

In alternative embodiments, the invention provides methods of reducing levels of protein O-GlcNAc modification in animal subjects, such as, veterinary and human subjects. In alternative embodiments, the invention provides methods of inhibiting an OGT in animal subjects, such as, veterinary and human subjects.

In specific embodiments, the invention provides compounds described generally by Formula (I) and the salts, prodrugs, and stereoisomeric forms thereof:

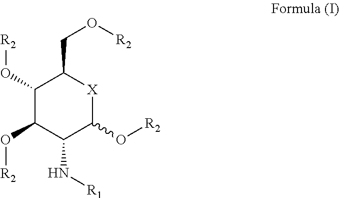

As set forth in Formula (I):

X may be S, Se or CH₂;

R₁ may be either H or C(O)R₄, where R₄ may be H, CH₂OH, CH₂N₃, CH₂SH, small branched or unbranched alkyl groups (such as methyl, ethyl, propyl, butyl, isopropyl, isobutyl, tert-butyl, valeryl, isovaleryl, etc.), ethers such as O-methyl, O-ethyl, O-propyl, O-isopropyl, O-butyl, O-isobutyl, etc.); thioethers such as S-methyl, S-ethyl, S-propyl, S-isopropyl, S-butyl, S-isobutyl, etc.; ureas; thioureas; etc., where the alkyl groups, ethers, thioethers, ureas, or thioureas may be optionally substituted;

R₂ may be H or C(O)R₅, where R₅ may be may be optionally substituted alkyl, alkenyl, alkynyl, aryl, or heteroaryl. In these cases, the ester groups may be hydrolyzed in vivo (e.g. in bodily fluids and/or within cells), releasing the active compounds in which R² is H. Prodrug embodiments of the invention include compounds where one, two, three or four of R₂ is C(O)CH₃;

[symbol] may be the alpha anomer, the beta anomer, or both.

In some embodiments of the invention, X may be S, R₂ may be H or Ac, and R₄ may be CH₃, CH₂CH₃, (CH₂)₂CH₃, CH(CH₃)₂, (CH₂)₃CH₃, CH₂CH(CH₃)₂, (CH₂)₄CH₃, C(CH₃)₃, CH₂C₆H₁₁, CH₂CᵢoH₈, CH₂CH(CH₃)CH₂CH₃, (CH₂)₃CH(CH₃)₂, etc.

In some embodiments of the invention, one or more of the following compounds: 5SGlcNAc, Ac-5SGlcNAc, 5CH₂GlcNAc, UDP-5CH₂GlcNAc, 5SGlcNAz, Ac-5SGlcNAz, are specifically excluded from the compounds described in Formula (I). In some embodiments of the invention, specific stereoisomers or enantiomers of one or more of the following compounds: 5SGlcNAc, Ac-5SGlcNAc, 5CH₂GlcNAc, UDP-5CH₂GlcNAc, 5SGlcNAz, Ac-5SGlcNAz, are specifically excluded from the compounds described in Formula (I).

In alternative embodiments, Formulae II V show sugar structures that can be used to target GTs:

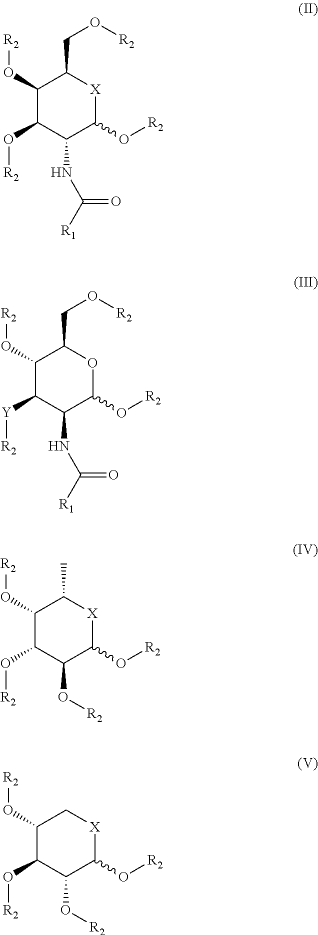

R₁ may be either H or C(O)R₄, where R₄ may be H, CH₂OH, CH₂N₃, CH₂SH, small branched or unbranched alkyl groups (such as methyl, ethyl, propyl, butyl, isopropyl, isobutyl, tert-butyl, valeryl, isovaleryl, etc.), ethers such as O-methyl, O-ethyl, O-propyl, O-isopropyl, O-butyl, O-isobutyl, etc.); thioethers such as S-methyl, S-ethyl, S-propyl, S-isopropyl, S-butyl, S-isobutyl, etc.; ureas; thioureas; etc., where the alkyl groups, ethers, thioethers, ureas, or thioureas may be optionally substituted;

R₂ may be H or C(O)R₅, where R₅ may be optionally substituted alkyl, alkenyl, alkynyl, aryl, or heteroaryl. In these cases, the ester groups may be hydrolyzed in vivo (e.g. in bodily fluids and/or within cells), releasing the active compounds in which R² is H. Prodrug embodiments of the invention include compounds where one, two, three or four of R₂ is C(O)CH₃;

[symbol] may be the alpha anomer, the beta anomer, or both.

As set forth in Formulae (II, IV and V):

X may be S, Se or CH₂;

As set forth in Formula III:

Y may be S or Se.

In alternative embodiments, Formulae VIX show derivatized sugar phosphate structures that could be used to target GTs:

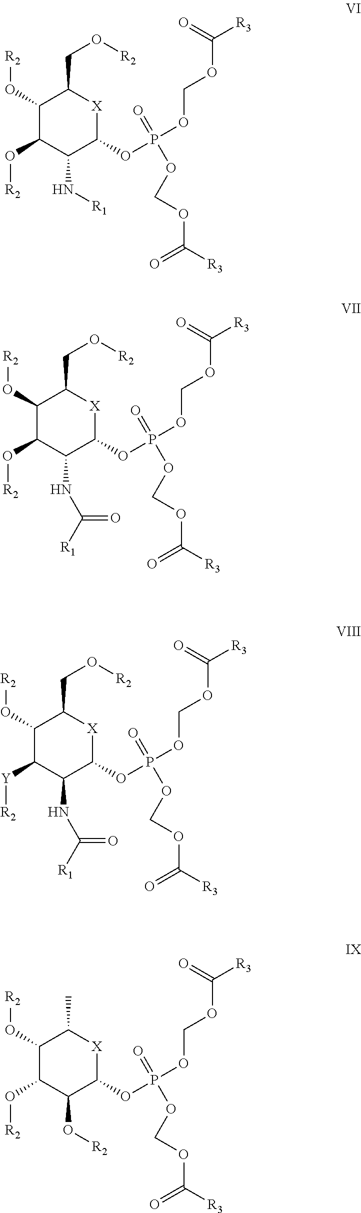

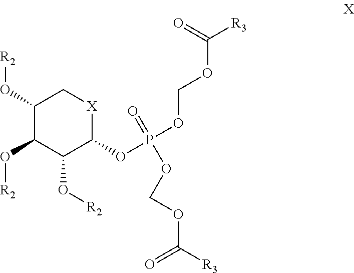

As set forth in Formulae (VI-X):

R₁ may be either H or C(O)R₄, where R₄ may be H, CH₂OH, CH₂N₃, CH₂SH, small branched or unbranched alkyl groups (such as methyl, ethyl, propyl, butyl, isopropyl, isobutyl, tert-butyl, valeryl, isovaleryl, etc.), ethers such as O-methyl, O-ethyl, O-propyl, O-isopropyl, O-butyl, O-isobutyl, etc.); thioethers such as S-methyl, S-ethyl, S-propyl, S-isopropyl, S-butyl, S-isobutyl, etc.; ureas; thioureas; etc., where the alkyl groups, ethers, thioethers, ureas, or thioureas may be optionally substituted;

R₂ may be H or C(O)Rₛ, where R₅ may be optionally substituted alkyl, alkenyl, alkynyl, aryl, or heteroaryl. In these cases, the ester groups may be hydrolyzed in vivo (e.g. in bodily fluids and/or within cells), releasing the active compounds in which R₂ is H. Prodrug embodiments of the invention include compounds where one, two, or three of R₂ is C(O)CF1₃;

R₃ may be H or C(O)R₆, where R₆ may be optionally substituted alkyl, alkenyl, alkynyl, aryl, or heteroaryl.

As set forth in Formulae (VI, VII, IX and X):

X may be S, Se or CH₂;

As set forth in Formula VIII:

Y may be S or Se.

“Alkyl” refers to a straight or branched hydrocarbon chain group consisting solely of carbon and hydrogen atoms, containing no unsaturation and including, for example, from one to ten carbon atoms, and which is attached to the rest of the molecule by a single bond. Unless stated otherwise specifically in the specification, the alkyl group may be optionally substituted by one or more substituents as described herein. Unless stated otherwise specifically herein, it is understood that the substitution can occur on any carbon of the alkyl group.

“Cycloalkyl” refers to a stable monovalent monocyclic, bicyclic or tricyclic hydrocarbon group consisting solely of carbon and hydrogen atoms, having for example from 3 to 15 carbon atoms, and which is saturated and attached to the rest of the molecule by a single bond, such as cyclopropyl, cyclobutyl, cyclopentyl, cyclohexyl, cycloheptyl, etc. Unless otherwise stated specifically herein, the term “cycloalkyl” is meant to include cycloalkyl groups which are optionally substituted as described herein.

“Alkenyl” refers to a straight or branched hydrocarbon chain group consisting solely of carbon and hydrogen atoms, containing at least one double bond and including, for example, from two to ten carbon atoms, and which is attached to the rest of the molecule by a single bond or a double bond. Unless stated otherwise specifically in the specification, the alkenyl group may be optionally substituted by one or more substituents as described herein. Unless stated otherwise specifically herein, it is understood that the substitution can occur on any carbon of the alkenyl group.

“Alkynyl” refers to a straight or branched hydrocarbon chain group consisting solely of carbon and hydrogen atoms, containing at least one triple bond and including, for example, from two to ten carbon atoms. Unless stated otherwise specifically in the specification, the alkynyl group may be optionally substituted by one or more substituents as described herein.

“Aryl” refers to a a single or fused aromatic ring group , including for example, 5-12 members, such as a phenyl or naphthyl group. Unless stated otherwise specifically herein, the term “aryl” is meant to include aryl groups optionally substituted by one or more substituents as described herein.

“Heteroaryl” refers to a single or fused aromatic ring group containing one or more heteroatoms in the ring, for example N, O, S, including for example, 5-14 members. Examples of heteroaryl groups include furan, thiophene, pyrrole, oxazole, thiazole, imidazole, pyrazole, isoxazole, isothiazole, 1,2,3-oxadiazole, 1,2,3-triazole, 1,2,4-triazole, 1,3,4-thiadiazole, tetrazole, pyridine, pyridazine, pyrimidine, pyrazine, 1,3,5-triazine, imidazole, benzimidazole, benzoxazole, benzothiazole, indolizine, indole, isoindole, benzofuran, benzothiophene, 1H-indazole, purine, 4H-quinolizine, quinoline, isoquinoline, cinnoline, phthalazine, quinazoline, quinoxaline, 1,8-naphthyridine, pteridine. Unless stated otherwise specifically herein, the term “heteroaryl” is meant to include heteroaryl groups optionally substituted by one or more substituents as described herein.

“Ether” refers to a compound in which an oxygen atom is bonded to an alkyl or an aryl group, or to two alkyl or two aryl groups, as described herein.

“Thioether” refers to a compound in which a sulphur atom is bonded to an alkyl or an aryl group, or to two alkyl or two aryl groups, as described herein.

“Optional” or “optionally” means that the subsequently described event of circumstances may or may not occur, and that the description includes instances where said event or circumstance occurs and instances in which it does not. For example, “optionally substituted alkyl” means that the alkyl group may or may not be substituted and that the description includes both substituted alkyl groups and alkyl groups having no substitution. Examples of optionally substituted alkyl groups include, without limitation, methyl, ethyl, propyl, etc. or including cycloalkyls such as cyclopropyl, cyclobutyl, cyclopentyl, cyclohexyl, cycloheptyl, etc., or including aryls such as phenyl or naphthyl; examples of optionally substituted alkenyl groups include allyl, crotyl, 2-pentenyl, 3-hexenyl, 2-cyclopentenyl, 2-cyclohexenyl, 2-cyclopentenylmethyl, 2-cyclohexenylmethyl, etc. In some embodiments, optionally substituted alkyl and alkenyl groups include C₁₋₆ alkyls or alkenyls.

As used herein the singular forms “a”, “and”, and “the” include plural referents unless the context clearly dictates otherwise. For example, “a compound” refers to one or more of such compounds, while “the enzyme” or “the glycosyltransferase” includes a particular enzyme as well as other family members and equivalents thereof as known to those skilled in the art.

Throughout this application, it is contemplated that the term “compound” or “compounds” refers to the compounds discussed herein and includes precursors and derivatives of the compounds, including acyl-protected derivatives, and pharmaceutically acceptable salts of the compounds, precursors, and derivatives. The invention also includes prodrugs of the compounds, pharmaceutical compositions including the compounds and a pharmaceutically acceptable carrier, and pharmaceutical compositions including prodrugs of the compounds and a pharmaceutically acceptable carrier.

In some embodiments, the formulations, preparation, and compositions including compounds according to the invention include mixtures of anomers (alpha and beta) or include individual anomers (alpha or beta). In general, the compound may be supplied in any desired degree of purity.

### Therapeutic Indications

The invention provides methods of treating conditions that are modulated, directly or indirectly, by a GT such as N-acetylglucosaminyltransferases, N-acetylgalactosaminyltransferases, fucosyltransferases, xylosyltransferases, sialyltransferases, mannosyltransferases, glucosyltransferases or galactosyltransferases, or by sugar-modified protein levels, for example, a conditions which require or respond to inhibition of GT activity.

In some embodiments, the invention provides methods of treating conditions that are modulated, directly or indirectly, by an OGT or by O-GlcNAc-modified protein levels, for example, a condition that is benefited by inhibition of an OGT or by a reduction of O-GlcNAc-modified protein levels. Such conditions include, without limitation, cancer, diabetes, insulin resistance, complications of diabetes, or autoimmune disease. The compounds of the invention are also useful in the treatment of diseases or disorders related to deficiency or over-expression of OGT or accumulation or depletion of O-GlcNAc, or any disease or disorder responsive to glycosyltransferase inhibition therapy. Such diseases and disorders include, but are not limited to, cancer, diabetes, insulin resistance, complications of diabetes, or autoimmune disease.

Such diseases and disorders may also include diseases or disorders related to the accumulation or deficiency in the enzyme O-GlcNAcase. Also included is a method of protecting or treating target cells expressing proteins that are modified by O-GlcNAc residues, the dysregulation of which modification results in disease or pathology.

In some embodiments, the invention provides methods of treating conditions that are modulated, directly or indirectly, by an FUT, for example, a condition that is benefited by inhibition of an FUT. Such conditions include, without limitation, inflammation, automimmune disorders, cancer (e.g., tumour growth and/or metastasis), etc. The compounds of the invention are also useful in the treatment of diseases or disorders related to over-expression of an FUT, or any disease or disorder responsive to fucosyltransferase inhibition therapy. Such diseases and disorders include, but are not limited to, inflammation, automimmune disorders, cancer (e.g., tumour growth and/or metastasis), infections, (e.g., Helicobacter pylori infections,) glaucoma, atherosclerosis, etc.

By a “cancer” or “neoplasm” is meant any unwanted growth of cells serving no physiological function. In general, a cell of a neoplasm has been released from its normal cell division control, i.e., a cell whose growth is not regulated by the ordinary biochemical and physical influences in the cellular environment. In most cases, a neoplastic cell proliferates to form a clone of cells which are either benign or malignant. Examples of cancers or neoplasms include, without limitation, transformed and immortalized cells, tumours, and carcinomas such as breast cell carcinomas and prostate carcinomas. The term cancer includes cell growths that are technically benign but which carry the risk of becoming malignant. By “malignancy” is meant an abnormal growth of any cell type or tissue. The term malignancy includes cell growths that are technically benign but which carry the risk of becoming malignant. This term also includes any cancer, carcinoma, neoplasm, neoplasia, or tumor.

Most cancers fall within three broad histological classifications: carcinomas, which are the predominant cancers and are cancers of epithelial cells or cells covering the external or internal surfaces of organs, glands, or other body structures (e.g., skin, uterus, lung, breast, prostate, stomach, bowel), and which tend to metastasize; sarcomas, which are derived from connective or supportive tissue (e.g., bone, cartilage, tendons, ligaments, fat, muscle); and hematologic tumors, which are derived from bone marrow and lymphatic tissue. Carcinomas may be adenocarcinomas (which generally develop in organs or glands capable of secretion, such as breast, lung, colon, prostate or bladder) or may be squamous cell carcinomas (which originate in the squamous epithelium and generally develop in most areas of the body). Sarcomas may be osteosarcomas or osteogenic sarcomas (bone), chondrosarcomas (cartilage), leiomyosarcomas (smooth muscle), rhabdomyosarcomas (skeletal muscle), mesothelial sarcomas or mesotheliomas (membranous lining of body cavities), fibrosarcomas (fibrous tissue), angiosarcomas or hemangioendotheliomas (blood vessels), liposarcomas (adipose tissue), gliomas or astrocytomas (neurogenic connective tissue found in the brain), myxosarcomas (primitive embryonic connective tissue), or mesenchymous or mixed mesodermal tumors (mixed connective tissue types). Hematologic tumors may be myelomas, which originate in the plasma cells of bone marrow; leukemias which may be “liquid cancers” and are cancers of the bone marrow and may be myelogenous or granulocytic leukemia (myeloid and granulocytic white blood cells), lymphatic, lymphocytic, or lymphoblastic leukemias (lymphoid and lymphocytic blood cells) or polycythemia vera or erythremia (various blood cell products, but with red cells predominating); or lymphomas, which may be solid tumors and which develop in the glands or nodes of the lymphatic system, and which may be Hodgkin or Non-Hodgkin lymphomas. In addition, mixed type cancers, such as adenosquamous carcinomas, mixed mesodermal tumors, carcinosarcomas, or teratocarcinomas also exist.

Cancers may also be named based on the organ in which they originate i.e., the “primary site,” for example, cancer of the breast, brain, lung, liver, skin, prostate, testicle, bladder, colon and rectum, cervix, uterus, etc. This naming persists even if the cancer metastasizes to another part of the body, that is different from the primary site. Cancers named based on primary site may be correlated with histological classifications. For example, lung cancers are generally small cell lung cancers or non-small cell lung cancers, which may be squamous cell carcinoma, adenocarcinoma, or large cell carcinoma; skin cancers are generally basal cell cancers, squamous cell cancers, or melanomas. Lymphomas may arise in the lymph nodes associated with the head, neck and chest, as well as in the abdominal lymph nodes or in the axillary or inguinal lymph nodes. Identification and classification of types and stages of cancers may be performed by using for example information provided by the Surveillance, Epidemiology, and End Results (SEER) Program of the National Cancer Institute.

“Diabetes” or “diabetes mellitus” is a group of metabolic diseases characterized by high blood sugar (glucose) levels, that result from defects in insulin secretion, or action, or both. Complications of diabetes are conditions or disorders as a consequence of diabetes or commonly found in diabetes patients and include without limitation, microvascular and macrovascular disease, blindness, kidney failure, nerve damage, atherosclerosis, leading to strokes, coronary heart disease, insulin resistance, vascular damage, skin ulcers, circulatory damage, diabetic nephropathy, diabetic retinopathy, diabetic neuropathy, etc.

“Inflammation” refers to a non-specific immune response caused, for example, by infection, irritatants (such as chemical irritants) or tissue or cell injury. Inflamation may be acute or chronic. Inflammatory diseases or disorders include, without limitation, atherosclerosis, allergy, inflammatory myopathy, cancer, inflammation caused by drugs or chemical, etc.

Autoimmunity generally refers to a misdirected immune response that occurs when the immune system goes awry and attacks the body itself. While autoimmunity may be generally present in most individuals and may be benign, in some cases a progression to a pathogenic state causes autoimmune disease. Autoimmunity is generally evidenced by the presence of antibodies or T lymphcytes reactive against host antigens. Autoimmune diseases include without limitation Type I diabetes mellitus, multiple sclerosis, systemic lupis erythematosus, Grave's disease, rheumatoid arthritis, chronic thyroiditis, etc.

The term “treating” as used herein includes treatment, prevention, and amelioration.

In alternative embodiments, the invention provides methods of reducing levels of protein O-GlcNAc modification in animal subjects, such as, veterinary and human subjects. This reduction of O-GlcNAc levels can be useful, for example, for the prevention or treatment of cancer, diabetes, insulin resistance, complications of diabetes, or autoimmune disease.

In alternative embodiments, the invention provides methods of inhibiting a GT, for example, an OGT or an FUT, in animal subjects, such as veterinary and human subjects.

In general, the methods of the invention are effected by administering a compound according to the invention to a subject in need thereof, or by contacting a cell or a sample with a compound according to the invention, for example, a pharmaceutical composition comprising a therapeutically effective amount of the compound according to Formulae (I-X). In some embodiments, compounds according to Formula I or VI are useful in the treatment of a disorder in which the regulation of O-GlcNAc protein modification is implicated, or any condition as described herein. Disease states of interest include without limitation, cancer, diabetes, insulin resistance, complications of diabetes, inflammation, automimmune disorders, cancer, infections, (e.g., Helicobacter pylori infections,) glaucoma, atherosclerosis, etc.

### Pharmaceutical & Veterinary Compositions, Dosages, And Administration

Pharmaceutical compositions including compounds according to the invention, or for use according to the invention, are contemplated as being within the scope of the invention. In some embodiments, pharmaceutical compositions including an effective amount of a compound of one or more of Formula (I-X) are provided.

The compounds of one or more of Formula (I-X) and their pharmaceutically acceptable salts, anomers, solvates, and derivatives are useful to because they have pharmacological activity in animals, including humans. In some embodiments, the compounds according to the invention are stable in plasma, when administered to a subject.

In some embodiments, compounds according to the invention, or for use according to the invention, may be provided in combination with any other active agents or pharmaceutical compositions where such combined therapy is useful to modulate OGT activity, for example, to treat cancer, diabetes, insulin resistance, complications of diabetes, inflammation, or autoimmune disease or any condition described herein or known as useful in the modulation of OGT activity.

In some embodiments, compounds according to the invention, or for use according to the invention, may be provided in combination with one or more agents useful in the prevention or treatment of cancer, diabetes, insulin resistance, complications of diabetes, inflammation, automimmune disorders, cancer, infections, (e.g., Helicobacter pylori infections,) glaucoma, atherosclerosis, etc.

It is to be understood that combination of compounds according to the invention, or for use according to the invention, with GT inhibitors is not limited to the examples described herein, but includes combination with any agent useful for the treatment of cancer, diabetes, insulin resistance, complications of diabetes, inflammation, or autoimmune disease. Combination of compounds according to the invention, or for use according to the invention, and other cancer or diabetes therapies may be administered separately or in conjunction. The administration of one agent may be prior to, concurrent to, or subsequent to the administration of other agent(s).

In alternative embodiments, the compounds may be supplied as “prodrugs” or protected forms, which release the compound after administration to a subject. For example, the compound may carry a protective group which is split off by hydrolysis in body fluids, e.g., in the bloodstream, thus releasing the active compound or is oxidized or reduced in body fluids to release the compound. Accordingly, a “prodrug” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a subject.

The term “prodrug” is also meant to include any covalently bonded carriers which release the active compound of the invention in vivo when such prodrug is administered to a subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol and acetamide, formamide, and benzamide derivatives of amine functional groups in the compounds of the invention and the like.

A discussion of prodrugs may be found in “Smith and Williams’ Introduction to the Principles of Drug Design,” H. J. Smith, Wright, Second Edition, London (1988); Bundgard, H., Design of Prodrugs (1985), pp. 7-9, 21-24 (Elsevier, Amsterdam); The Practice of Medicinal Chemistry, Camille G. Wermuth et al., Ch 31, (Academic Press, 1996); A Textbook of Drug Design and Development, P. Krogsgaard-Larson and H. Bundgaard, eds. Ch 5, pgs 113 191 (Harwood Academic Publishers, 1991); Higuchi, T., et al., “Pro-drugs as Novel Delivery Systems,” A.C.S. Symposium Series, Vol. 14; or in Bioreversible Carriers in Drug Design, ed. Edward B. Roche, American Pharmaceutical Association and Pergamon Press, 1987, all of which are incorporated in full by reference herein.

Suitable prodrug forms of the compounds of the invention include embodiments in which R₂ is C(O)R, where R is optionally substituted alkyl, alkenyl, alkynyl, aryl, or heteroaryl. In these cases the ester groups may be hydrolyzed in vivo (e.g. in bodily fluids), releasing the active compounds in which R is H. Prodrug embodiments of the invention include compounds of Formula (I-X) where one, two, three or four of R₂ is C(O)CH₃. In some embodiments, suitable prodrug forms of the compounds of the invention include 1-phosphate derivatized compounds as described for example in Formula (VI-X).

Compounds according to the invention, or for use according to the invention, can be provided alone or in combination with other compounds in the presence of a liposome, an adjuvant, or any pharmaceutically acceptable carrier, diluent or excipient, in a form suitable for administration to a subject such as a mammal, for example, humans, cattle, sheep, etc. If desired, treatment with a compound according to the invention may be combined with more traditional and existing therapies for the therapeutic indications described herein. Compounds according to the invention may be provided chronically or intermittently. “Chronic” administration refers to administration of the compound(s) in a continuous mode as opposed to an acute mode, so as to maintain the initial therapeutic effect (activity) for an extended period of time. “Intermittent” administration is treatment that is not consecutively done without interruption, but rather is cyclic in nature. The terms “administration,” “administrable,” or “administering” as used herein should be understood to mean providing a compound of the invention to the subject in need of treatment.

“Pharmaceutically acceptable carrier, diluent or excipient” includes without limitation any adjuvant, carrier, excipient, glidant, sweetening agent, diluent, preservative, dyecolorant, flavor enhancer, surfactant, wetting agent, dispersing agent, suspending agent, stabilizer, isotonic agent, solvent, or emulsifier that has been approved, for example, by the United States Food and Drug Administration or other governmental agency as being acceptable for use in humans or domestic animals.

The compounds of the present invention may be administered in the form of pharmaceutically acceptable salts. In such cases, pharmaceutical compositions in accordance with this invention may comprise a salt of such a compound, preferably a physiologically acceptable salt, which are known in the art. In some embodiments, the term “pharmaceutically acceptable salt” as used herein means an active ingredient comprising compounds of Formula I-X used in the form of a salt thereof, particularly where the salt form confers on the active ingredient improved pharmacokinetic properties as compared to the free form of the active ingredient or other previously disclosed salt form.

A “pharmaceutically acceptable salt” includes both acid and base addition salts. A “pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

A “pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine,methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Thus, the term “pharmaceutically acceptable salt” encompasses all acceptable salts including but not limited to acetate, lactobionate, benzenesulfonate, laurate, benzoate, malate, bicarbonate, maleate, bisulfate, mandelate, bitartarate, mesylate, borate, methylbromide, bromide, methylnitrite, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphatediphosphate, gluceptate, polygalacturonate, gluconate, salicylate, glutame, stearate, glycollylarsanilate, sulfate, hexylresorcinate, subacetate, hydradamine, succinate, hydrobromide, tannate, hydrochloride, tartrate, hydroxynaphthoate, teoclate, iodide, tosylate, isothionate, triethiodide, lactate, panoate, valerate, and the like.

Pharmaceutically acceptable salts of the compounds of the present invention can be used as a dosage for modifying solubility or hydrolysis characteristics, or can be used in sustained release or prodrug formulations. Also, pharmaceutically acceptable salts of the compounds of this invention may include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide.

Pharmaceutical formulations will typically include one or more carriers acceptable for the mode of administration of the preparation, be it by injection, inhalation, topical administration, lavage, or other modes suitable for the selected treatment. Suitable carriers are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The table or capsule may be enteric coated, or in a formulation for sustained release. Many suitable formulations are known, including, polymeric or protein microparticles encapsulating a compound to be released, ointments, gels, hydrogels, or solutions which can be used topically or locally to administer a compound. A sustained release patch or implant may be employed to provide release over a prolonged period of time. Many techniques known to skilled practitioners are described in Remington: the Science & Practice of Pharmacy⁷⁷. Formulations for parenteral administration may, for example, contain excipients, polyalkylene glycols such as polyethylene glycol, oils of vegetable origin, or hydrogenated naphthalenes. Biocompatible, biodegradable lactide polymer, lactideglycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The compounds or pharmaceutical compositions according to the present invention may be administered by oral or non-oral, e.g., intramuscular, intraperitoneal, intravenous, intracisternal injection or infusion, subcutaneous injection, transdermal or transmucosal routes. In some embodiments, compounds or pharmaceutical compositions in accordance with this invention or for use in this invention may be administered by means of a medical device or appliance such as an implant, graft, prosthesis, stent, etc. Implants may be devised which are intended to contain and release such compounds or compositions. An example would be an implant made of a polymeric material adapted to release the compound over a period of time. The compounds may be administered alone or as a mixture with a pharmaceutically acceptable carrier e.g., as solid formulations such as tablets, capsules, granules, powders, etc.; liquid formulations such as syrups, injections, etc.; injections, drops, suppositories, pessaries. In some embodiments, compounds or pharmaceutical compositions in accordance with this invention or for use in this invention may be administered by inhalation spray, nasal, vaginal, rectal, sublingual, or topical routes and may be formulated, alone or together, in suitable dosage unit formulations containing conventional non-toxic pharmaceutically acceptable carriers, adjuvants and vehicles appropriate for each route of administration.

The compounds of the invention may be used to treat animals, including mice, rats, horses, cattle, sheep, dogs, cats, and monkeys. However, compounds of the invention can also be used in other organisms, such as avian species (e.g., chickens). The compounds of the invention may also be effective for use in humans. The term “subject” or alternatively referred to herein as “patient” is intended to be referred to an animal, preferably a mammal, most preferably a human, who has been the object of treatment, observation or experiment. However, the compounds, methods and pharmaceutical compositions of the present invention may be used in the treatment of animals. Accordingly, as used herein, a “subject” may be a human, non-human primate, rat, mouse, cow, horse, pig, sheep, goat, dog, cat, etc. The subject may be suspected of having or at risk for having a condition requiring modulation of a GT activity, e.g., O-GlclAcase activity or inhibition of OGT or FUT activity.

An “effective amount” of a compound according to the invention includes a therapeutically effective amount or a prophylactically effective amount. A “therapeutically effective amount” refers to an amount effective, at dosages and for periods of time necessary, to achieve the desired therapeutic result, such as inhibition of a GT, such as an FUT or OGT, reduction of O-GlcNAc levels, or treatment of any condition described herein. A therapeutically effective amount of a compound may vary according to factors such as the disease state, age, sex, and weight of the individual, and the ability of the compound to elicit a desired response in the individual.

Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as inhibition of a GT, such as an FUT or OGT, reduction of O-GlcNAc levels, or prevention of any condition described herein. Typically, a prophylactic dose is used in subjects prior to or at an earlier stage of disease, so that a prophylactically effective amount may be less than a therapeutically effective amount. A suitable range for therapeutically or prophylactically effective amounts of a compound may be any value from 0.1 nM-0.1M, 0.1 nM-0.05M, 0.05 nM-15μM or 0.01 nM-10 μM.

In alternative embodiments, in the treatment or prevention of conditions which require modulation of a GT activity, such as an FUT or OGT activity, an appropriate dosage level will generally be about 0.01 to 1000 mg per kg subject body weight per day, and can be administered in singe or multiple doses. In some embodiments, the dosage level will be about 0.1 to about 250 mg/kg per day. It will be understood that the specific dose level and frequency of dosage for any particular patient may be varied and will depend upon a variety of factors including the activity of the specific compound used, the metabolic stability and length of action of that compound, the age, body weight, general health, sex, diet, mode and time of administration, rate of excretion, drug combination, the severity of the particular condition, and the patient undergoing therapy.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage. In general, compounds of the invention should be used without causing substantial toxicity, and as described herein, the compounds exhibit a suitable safety profile for therapeutic use. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

Various alternative embodiments and examples of the invention are described herein. These embodiments and examples are illustrative and should not be construed as limiting the scope of the invention.

The present invention will be further illustrated in the following examples.

### EXAMPLE I

### Materials and Methods

Synthesis of (Ac)5SGlcNAc: Synthesis of (Ac-)5SGlcNAc was performed as described previously²⁷ with some minor modifications. A full description of the methods used and characterization of the compounds are in Example II.

Chemoenzymatic synthesis of UDP-5SGlcNAc: Over-expression and purification of GNK, AGM, and AGX1 were carried out as described previously³⁴⁻³⁶. The cloning, over-expression and purification of UDP-GlcNAc 4-epimerase was performed according to standard molecular biology procedures. Inorganic pyrophosphatase (PPA), ATP and UTP were purchased from Sigma. GNK, AGM, and AGX1 were buffer exchanged into 50 mM Tris, pH 7.5, 2 mM MgCl₂ using PD10 columns (GE Healthcare), and all substrates were dissolved in the same buffer. GDP-Glc, at a concentration of 68 μM was included as an internal standard to which CE peaks were normalized.

To synthesize UDP-5SGlcNAc, a one-pot reaction consisting of 2.5 mM ATP, 1 mM UTP, 0.6 mM 5SGlcNAc, 0.4 μM GNK, 2.9 μM AGM, 0.3 μM AGX and 50 mU/mL PPA was incubated for 14 h at 37° C. The reaction mixture was treated for an additional 4 h at 37° C. with calf alkaline phosphatase (Roche). Small aliquots (˜100 μL) of sample were run by CE (see below for details) to monitor UDP-5SGlcNAc production. Epimerization of UDP-5SGlcNAc was tested by spiking an aliquot with UDP-GlcNAc 4-epimerase and incubating for 14 h at 37° C.

Purification of UDP-5SGlcNAc: UDP-5SGlcNAc was purified by ion exchange chromatography and HPLC. More specifically, enzymes were removed by passing the mixture through a centrifugal filtration device (10 kDa molecular weight cut-off; Centricon). The filtrate containing the desired product was desalted by passing it over a column packed with a 7 mL bed volume of Dowex AG 1-X4 ion exchange resin (BioRad), previously converted into the formate form and then pre-equilibrated in water. After loading, the column was washed, at a flow rate of 2 mL/min, with 10 bed volumes of H₂O followed by 10 bed volumes 4 M formic acid, and eluted with 10 bed volumes of 550 mM ammonium formate, pH 4.0³⁷. The fraction containing UDP-5SGlcNAc was concentrated in vacuo and further purified by HPLC on a Hewlett Packard series 1100 instrument, equipped with an Eclipse XDB-C18 (5 μm, 9.4×250 mm) column (Agilent Technologies), using ion-paired conditions (adapted from Ref.³⁸). Compounds were detected by monitoring the UV absorbance at 254 nm and UV-active peaks were assessed for purity by HPLC and CE. Fractions containing the desired product were pooled and lyophilized.

Capillary electrophoresis (CE): CE was performed on a ProteomeLab PA800 (Beckman-Coulter) using fused silica capillaries of 50 μM internal diameter×44 cm (to detector). The running buffer was 40 mM Na₂B₄O₇ (Sigma), pH 9.5, containing 1.0% (wv) polyethylene glycol (MW 20,000; Fluka) and filtered prior to use. The capillary was conditioned by washing with 1 N NaOH (2 min, 20 psi), 18 MΩ H₂O (3 min, 20 psi) and running buffer (5 min, 40 psi). After injecting a short (15 nL) H₂O plug, samples were electrostatically introduced and pre-concentrated at the anode by applying a potential of -0.5 kV for 10 s according to the field-amplified sample injection technique described by Chien and Burgi³⁹. Electrophoresis was carried out at a constant voltage of 30 kV and capillary temperature of 22° C. Electropherograms were derived by measuring the absorbance at 254 (+/−10) nm at a rate of 4 Hz. Peaks were integrated using 32 Karat 5.0 software (Beckman-Coulter) and all peaks were normalized to the GDP-Glc internal standard.

OGT transfer: The ability of OGT (which was over-expressed and purified as described in Ref. ⁴⁰) to transfer UDP-5SGlcNAc was tested using recombinant nup62 as the acceptor. The sub-cloning, protein over-expression and purification of nup62 was performed according to standard molecular biology procedures. Assays contained 30 μM nup62, 20 μM UDP-(5S)GlcNAc and 0.4 μM OGT in 20 mM phosphate, pH 7.4, 150 mM NaCl (phosphate buffered saline; PBS), to in a total volume of 100 μL, and were allowed to proceed for an appropriate time between 10 mM and 2 h at 37° C. in order to maintain a constant rate without substrate depletion. Reactions were quenched upon addition of an equal volume of ethanol, frozen at -20° C. for 1 h to precipitate proteins and centrifuged at 13,000 rpm for 20 mM. 5 μM GDP-Glc, an internal standard, was added prior to freezing. The supernatant was removed and lyophilized. Nucleotides and nucleotide sugars were extracted as described below for the cell lysates, resuspended in 200 μL H₂O, and run by CE. Production of UDP was monitored at 254 nm; the concentration was determined from a standard curve of UDP standards, which were prepared in triplicate using the same procedure as the reactions, at a concentration of between 1 and 20 Controls in the absence of nup62 were subtracted to account for OGT-catalyzed hydrolysis of the UDP-sugars.

OGT inhibition: The ability of UDP-5SGlcNAc to inhibit OGT activity was assessed using radiolabelled [³H]GlcNAe-UDP (American Radiolabel) as the donor and recombinant nup62 as the acceptor. Assays contained 18 μM of nup62, 1 μM of UDP-GlcNAc (constant specific activity of 0.5 Ci/mmol of [³H]-UDP-GlcNAc), 100 nM OGT, and various concentrations (0-25 μM) of UDP-5SGlcNAc in PBS. The Vₘₐₓ for OGT was determined using high (30, 40 and 50 μM) concentrations of UDP-GlcNAc (containing 0.033, 0.025, 0.02 Ci/mmol specific activity, respectively). Reactions were incubated at 37° C. for 1 h (over which time OGT has been shown to retain constant activity). The assay mixture was applied to a 1.5×3 cm piece of nitrocellulose membrane (Bio-Rad) and allowed to air dry. Membranes were rinsed with five washes (total 100 mL) of PBS and air dried. The membranes were loaded into scintillation vials, and 4 mL of scintillation fluid (Amersham) was added. The tritium levels were quantified by liquid scintillation counter (BECKMAN LS6000). All reactions were performed in triplicate. The inhibitor concentration was plotted against the inverse of the rate, and the Kᵢ value was taken where the line of best fit intersected 1/Vₘₐₓ.

Cell culture: Cells were grown in an incubator at 37° C. and 5% CO₂ atmosphere. Media and serum were purchased from Invitrogen. COS-7 cells were cultured in Dulbecco's modified Eagle's medium (DMEM) with 5% fetal bovine serum (FBS), and were typically 80-100% confluent prior to experimentation. HepG2, SK-N-SH, and EMEG32^{-/-} cells were cultured in Dulbecco's modified Eagle's medium (DMEM) with 5% fetal bovine serum (FBS), CHO cells in DMEMF-12 with 5% FBS and PC12 cells in DMEM with 5% FBS and 5% horse serum. Prior to experimentation, PC12 cells were plated at a density of 3-4×10⁵ cells10 cm plate, which had been pre-treated with 5 μgcm² rat tail collagen (BD Biosciences). Cells were differentiated in DMEM containing 1% horse serum, in the presence of 2.5S mouse nerve growth factor (Chemicon) at a final concentration of 25 ng mL⁻¹, for 4-5 days prior to treatment. Other cell lines were 80-100% confluent prior to treatment. Following treatment with (Ac-)5SGlcNAc, using various conditions, cells were washed gently with PBS, lysed in SDS loading buffer and boiled for 10 minutes. Mouse hybridoma cells (obtained from the Developmental Studies Hybridoma Bank; antibody E7 for (:1-tubulin) were cultured in RPMI40 with 10% FBS. Prior to treatment with Ac-5SGlcNAc, hybridoma cells were cultured for 2 days in RPMI40 with 5% FBS, and during treatment in RPMI40 with no serum. Cells were collected, centrifuged (800 x g, 5 min) and the supernatant removed. Cells were washed with PBS, centrifuged, the pellet resuspended in SDS loading buffer and boiled. The supernatant after centrifugation, which contained the IgG antibody produced by the cells, was used for immunoprecipitation (see below for details).

Westernlectin blots: Samples were run on 10% SDS polyacrylamide gels and transferred onto nitrocellulose membranes (Biorad). Membranes were blocked for 1 h in PBS containing 0.1% Tween-20 (PBS-T) containing 1% (unless otherwise stated) bovine serum albumin (BSA), and probed with the appropriate primary antibody or lectin in 1% BSA in PBS-T overnight at 4° C. Blots were washed for 1 hr with PBS-T, and blocked for a further 30 min with 1% BSA in PBS-T at room temperature. They were then probed with the appropriate horseradish peroxidise (HRP)-conjugated secondary antibodyprobe in 1% BSA in PBS-T at room temperature for 1 h, followed by washing in PBS-T for 1 h. Blots were developed with SuperSignal West Pico Chemiluminescent Substrate (Pierce) and exposed to CL-XPosure film (Pierce). Densitometry was performed using ImageQuant 5.2 (Molecular Dynamics), and fits to the data were made using GRAFIT⁴¹.

Antibodieslectins: O-GlcNAc levels were assessed primarily using CTD110.6 (Covance), but RL2 (Abcam) and HGAC85 (Abcam) were also tested. Actin was probed using JLA20 (Developmental Studies Hybridoma Bank). OGA levels were assessed using a polyclonal anti-OGA antibody which was a kind gift from Dr. Gerald Hart and OGT levels using H-300 (Santa Cruz Biotechnology). nup62 was probed using mAb 414 (Covance). Secondary antibodies employed (HRP-conjugated goat anti-mouse IgM, goat anti-mouse IgG, goat anti-chicken IgY and goat anti-rabbit IgG) were obtained from Santa Cruz Biotechnology. Biotinylated-ConA, PHA-L, SNA and MAA were purchased from EY Laboratories, and biotinylated-GNA from Vector Laboratories. HRP-conjugated streptavidin (Pierce) was used as a probe for the biotinylated lectins.

Blocking antibody interactions: Recombinantly modified nup62 was run on a gel and transferred onto nitrocellulose membrane. CTD110.6 at the appropriate dilution was incubated with 0-3 mM Me-GlcNAc or Me-5SGlcNAc for 1 h prior to applying to the membrane overnight at 4° C. The standard procedure for western blots was subsequently followed.

OGA kinetics: Synthesis and characterization of para-methoxyphenyl-5SGlcNAc is described in Example II. Kinetics were performed at 37° C. in PBS, pH 7.4 in a total volume of 150 pt. Substrate concentrations of 25 μM to 1.5 mM were used for pMP-GlcNAc and 25 to 800 μM for pMP-5SGlcNAc. OGA (which was over-expressed and purified as described in Ref. ⁴²) was used at a concentration of 1 μM for pMP-GlcNAc and 3.6 μM for pMP-5SGlcNAc. Rates were monitored at a wavelength of 296 nm over the course of 10-20 min. Data were fit to the Michaelis-Menten equation in GRAFIT⁴¹ and the k_{cat} and K_{M} values determined.

Immunoprecipitation of nup62: nup62 was immunoprecipitated from COS-7 cell lysates using the procedure described in Ref.⁹. Following washing of the beads, and prior to elution, nup62 was incubated in the absence or presence of BtGH84 (overexpressed and purified as described previously⁴³) shaking for 2 h at room temperature, which would allow O-GlcNAc levels on nup62 to be assessed. nup62 was subsequently eluted from the beads upon addition of SDS loading buffer and boiled for 10 min.

Click-iT® kit for labelling O-GlcNAc residues: The ‘Click-iT® O-GlcNAc Enzymatic Labeling System’ was purchased from Invitrogen, and essentially the manufacturer's protocol was followed with some alterations. nup62 was immunoprecipitated and eluted from the beads in 1% SDS in 20 mM Hepes, pH 7.9, and boiled for 10 min; this was used as the substrate for the chemoenzymatic labeling using UDP-GalNAz and GalTl. Following the overnight reaction, biotinylated phosphine (in 20% DMF) was added at a final concentration of 50 μM and incubated at 37° C. for 90 mM. The reaction was stopped by the addition of SDS-PAGE loading buffer, and the samples were boiled for 10 min. Samples were run on a gel and transferred. The same procedure as described for the western blots was used, starting from the second blocking step. Blots were then probed with HRP-conjugated streptavidin (Pierce).

Extraction of nucleotide sugars from cell lysates: COS-7 cells, on 10 cm plates, were treated with 0, 50, 250 or 1000 μM Ac-5SGlcNAc for 24 h; each condition was performed in triplicate. Cells were washed gently with PBS prior to the addition of 1 mL PBS containing 10 mM EDTA. Cells were incubated at 37° C. for 4 mM, the cells scraped from the plate, and pelleted by centrifugation (1000 rpm, 10 min, 4° C.). Cell pellets were immediately lysed by the addition of 750 μL 75% (vv) ethanol (−20° C.) and sonicated using a W-375 ultrasonic processor (Heat Systems Ultrasonics, Inc.). Insoluble material was removed by centrifugation (14,000 rpm, 15 min, 4° C.) and the supernatant was snap frozen and lyophilized^{44,45}. The crude extract was spiked with GDP-Glc (200 pmol) as an internal standard, dissolved in 0.5 mL 18 MΩ H₂O and nucleotide sugars extracted using Envi-Carb graphite solid-phase extraction cartridges (200 mg) (Supelco) as described by Räbinä et al³⁸. Briefly, Envi-Carb cartridges were conditioned with 80% (vv) CH₃CN and 20% 0.1% (vv) TFA (3 mL), followed by 18 MΩ H₂O (6 mL), before samples were applied. After passing the samples through the SPE cartridge, the cartridge was sequentially washed with 2 mL of each of 18 MΩ H₂O, 25% (vv) CH₃CN, and 50 mM triethylammonium acetate (TEAA), pH 7.0. Sugar nucleotides were eluted with 3×1 mL 25% CH₃CN in 50 mM TEAA, pH 7.0, passed through a 0.22 μm filter (Millipore), lyophilized, and stored at −20° C. until analysis. Extracted sugar nucleotides were dissolved in 200 μL 18 MΩ H₂O and an aliquot was diluted 1:4 prior to characterization by CE using a method adapted from Feng et al⁴⁶.

Precipitation of antibody: Following collection of mouse hybridoma cells, cells were centrifuged and the supernatant (i.e. cell media containing the IgG antibody produced by the cells) was removed. 1 mL supernatant was incubated with 100 μL Protein GProtein-A agarose beads (Calbiochem) shaking overnight at 4° C. Beads were washed three times in PBS containing 0.1% NP-40. Antibody was eluted by the addition of SDS-PAGE loading buffer and boiling for 10 minutes. Assessment of total antibody precipitated was made by probing blots with secondary goat anti-mouse IgG antibody (using the western blot procedure starting from the second blocking step).

PNGase F cleavage: PNGase F was purchased from New England Biolabs and used as described in the manufacturer's protocol. Immunoprecipitated antibody (from the mouse hybridoma cells) was eluted from the beads in ‘10× denaturation buffer’ prior to PNGase F treatment.

### EXAMPLE II

### Synthesis and Characterization of Ac-5SGlcNAc, 5SGlcNAc, pMP-5SGlcNAc, Me-5SGlcNAc, and Ac-5SGlcNAz

Synthesis of Ac-5SGlcNAc (9) and 5SGlcNAc (10) was carried out following literature procedures with some modifications^{37,38}.

2-Acetamido-1, 3,4, 6-tetra-O-acetyl-2-deoxy-5-thio-a-D-glucopyranose (9)²⁷-¹H-NMR (500 MHz, CDCl₃) δ (ppm) 5.92 (d, J=3.04 Hz, 11⁻1), 5.70 (d, J=8.86 Hz, 1H), 5.37 (dd, J=10.74, 9.64 Hz, 1H), 5.16 (dd, J=10.89, 9.70 Hz, 1H), 4.63 (m, 1H), 4.34 (dd, J=12.11, 4.95 Hz, 1H), 4.03 (dd, J=12.11, 3.15 Hz, 1H), 3.50 (ddd, J=10.8, 4.80, 3.20, 1H), 2.17 (s, 3H), 2.05 (s, 3H), 2.03 (s, 3H), 2.02 (s, 3H), 1.90 (s, 3H); ¹³C-NMR (125 MHz, CDCl₃) δ (ppm) 171.73, 170.60, 169.52, 169.16, 168.77, 72.79, 71.78, 71.47, 61.10, 55.20, 39.75, 23.08, 21.14, 20.65, 20.53.

2-Acetamido-2-deoxy-5-thio-α-D-glucopyranose (10)²⁷-¹H-NMR (600 MHz, MeOH-d₄) δ (ppm) 4.89 (d, J=3.17 Hz, 1H), 4.07 (dd, J=10.50, 2.82 Hz, 1H), 3.87-3.79 (m, 2H), 3.65 (dd, J=10.46, 8.82 Hz, 1H), 3.55 (dd, J=10.37, 8.93 Hz, 1H), 3.25-3.22 (m, 1H), 1.96 (s, 3H);¹³C-NMR (150 MHz, MeOH-d₄) δ (ppm) 173.35, 76.88, 73.52, 73.50, 62.65, 60.01, 44.89, 22.72.

Synthesis of pMP-5SGlcNAc (14)

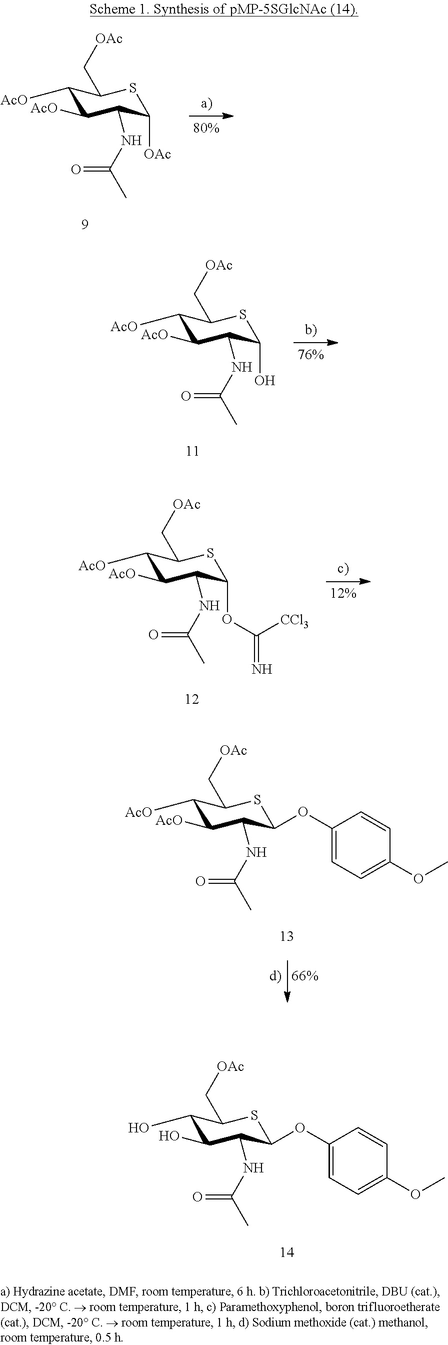

2-Acetamido-3,4,6-tri-O-acetyl-2-deoxy-5-thio-α-D-glucopyranose (11) Anomeric deacetylation was effected in a manner similar to that reported previously for different saccharides⁴⁸. To a solution of 9 (0.200 g, 0.494 mmol) in dimethylformamide (1 mL), stirring at room temperature, hydrazine acetate (0.050 g, 0.543 mmol) was added. The reaction was left to stir until all starting material was consumed (6 hours). The reaction was concentrated on the high vacuum rotary evaporator, and co-evaporated twice with toluene, without heating. The resulting residue was taken up in ethyl acetate and washed two times with saturated sodium bicarbonate, water (until a neutral pH was obtained) and brine. Each aqueous layer was back washed with ethyl acetate. The pooled organic layers were dried using magnesium sulphate, filtered and concentrated. The reaction was purified via flash column chromatography with a solvent system of 70% ethyl acetate, 30% hexanes, and subsequently concentrated on the high vacuum rotary evaporator to reveal a white solid. This was recrystallized in ethyl acetate and hexanes (0.144 g, 80%).¹H-NMR (600 MHz, CDCl₃) δ (ppm) 4.89 (dd, J=10.50, 2.82 Hz, 1H), 3.88 (m, 2H), 3.70 (dd, J=10.46, 8.82 Hz, 1H), 3.60 (dd, J=10.37, 8.93 Hz, 1H), 3.28 (m, 1H), 2.00 (s, 3H); ¹³C-NMR (150 MHz, CDCl₃) δ (ppm) 171.91, 75.43, 72.07, 72.05, 61.20, 58.56, 43.44, 21.27. HRMS (m/z): [M+H]⁺ calcd for C₁₄H₂₁NO₈S: 364.1066; found 364.1070

2-Acetamido-3,4,6-tri-O-acetyl-1-triehloroacetimidate-2-deoxy-5-thio-α-D-glucopyranose (12)—Formation of the trichloroacetrnidiate donor was carried out by modification of a previously described procedure used for different saccharides^{24,49-}⁵¹. 11 was dissolved in dichloromethane, and to this trichloroacetonitrile (0.181 g, 1.27 mmol) was added with a catalytic amount of DBU (˜1 drop) at 0° C. The reaction was allowed to stir at 0° C. until the reaction completion (0.5 hours). The reaction was diluted with benzene and concentrated on the high vacuum rotary evaporator to dryness. The crude mixture was purified using flash column chromatography using a solvent system of 60% ethyl acetate in hexanes. The pure product was isolated as a clear oil, which solidified to give a white solid (0.086 g, 76%). ¹H-NMR (600 MHz, CDO₃) δ (ppm) 8.77 (s, 1H), 6.08 (d, J=3.00 Hz, 1H), 5.79 (d, J=8.80 Hz, 1H), 5.37 (dd, J=10.68, 9.79 Hz, 1H), 5.20 (dd, J=10.68, 9.78 Hz, 1H), 4.65 (m, 1H), 4.29 (dd, J=12.14, 4.98 Hz, 1H), 4.00 (dd, J=12.11, 3.13 Hz, 1H), 3.49 (m, 1H), 2.00 (s, 3H), 1.99 (s, 3H), 1.98 (s, 3H);¹³C-NMR (150 MHz, CDCl₃) δ (ppm) 171.51, 170.49, 169.59, 169.21, 159.20, 90.79, 77.82, 71.69, 71.31, 60.99, 55.80, 40.14, 23.06, 20.63, 20.60, 20.52. HRMS (m/z): [M+H]⁺ caled for C₁₆H₁₂₁N₂O₈SCl₃: 507.0162; found 507.0173.

para-Methoxyphenyl 2-acetaando-3,4,6-tri-O-acetyl-2-deoxy-5-thio-α-D-glucopyranoside (13)—Glycosylation was effected by adaptation of a reported method used for glycosylation of trichloroacetimidate donors of other saccharides^{24,51}. A solution of 12 (0.050 g, 0.100 mmol) in dichloromethane (0.77mL) and paramethoxyphenol (0.025 g, 0.20 mmol) was stirred at -20° C. To this solution a catalytic amount of boron trifluoroetherate (˜1 drop) was added, and the reaction was immediately allowed to warm to room temperature. The reaction was stirred for 1 hour, at which time the reaction was complete. To quench the borontrifluoroetherate, 2 equivalents of triethylamine (0.026 mL) was added to the reaction mixture. The reaction was concentrated on the high vacuum rotary evaporator without heating. Flash column chromatography was performed in order to isolate the spots observed by TLC that were presumed to be the alpha and beta anomers. Upon obtaining ¹H NMR, it was determined that the two products isolated were the alpha and beta anomers, in a ratio of 70% alpha and 30% beta. The two anomers were separated via flash column chromatography, using a solvent system of 60% ethyl acetate in hexanes to give rise to pure beta-glycosylated product (0.056 g, 12%), and alpha-glycosylated product (0.096 g, 21%), giving an overall yield of 34%. ¹H-NMR (600 MHz, CDCl₃) δ (ppm) 6.89 (d, J=9.07 Hz, 2H), 6.77 (d, J=9.09 Hz, 2H), 5.88 (d, J=8.82 Hz, 1H), 5.31 (m, 1H), 5.13 (d, J=6.33 Hz, 1H), 5.04 (m, 1H), 4.52 (m, 1H), 4.28 (m, 2H), 3.70 (s, 3H), 3.14 (m, 1H), 2.02 (s, 3H), 2.01 (s, 3H), 1.99 (s, 3H), 1.92 (s, 3H); ¹³C-NMR (150 MHz, CDCl₃) δ (ppm) 170.55, 170.15, 169.70, 168.95, 155.44, 150.46, 118.20, 114.71, 80.57, 71.04, 69.16, 63.44, 55.67, 40.60, 29.72, 23.41, 20.81, 20.72, 20.70. HRMS (m/z): [M+H]⁺ calcd for C₂₁H₂₇N₂O₉S: 470.1485; found 470.1478.

para-Methoxyphenyl 2-acetamido-2-deoxy-5-thio-α-D-glucopyranoside (14)—In a solution of 13 (0.0062 g, 0.013 mmol) and methanol (0.25 mL), a catalytic amount of sodium methoxide was added (enough to bring the reaction solution to pH 10). The reaction was allowed to stir at room temperature. After twenty minutes of reaction, a white solid began to precipitate out of solution. The reaction had reached completion after 30 minutes. The reaction was first diluted with additional methanol, and subsequently brought to a neutral pH by the drop-wise addition of a dilute mixture of acetic acid in methanol (pH 4). The reaction was concentrated on a high vacuum rotary evaporator without heating. 14 was purified by recrystallization with ethanol and ether, resulting in a white crystalline product (0.0033 g, 66%). ¹H-NMR (600 MHz, MeOH, D₄) δ (ppm) 6.99 (d, J=9.16 Hz, 2H), 6.83 (d, J=9.16 Hz, 2H), 5.10 (d, J=9.29 Hz, 1H), 4.19 (dd, J=9.60Hz, 1H), 3.96 (dd, J=11.44, 3.73 Hz,1H), 3.79 (dd, J=11.44, 6.47 Hz, 1H), 3.74 (s, 311), 3.60 (dd, J=10.07 Hz, 1H), 3.38 (dd, J=9.89 Hz, 1H), 2.89 (m, 1H), 1.96 (s, 3H); ¹³C-NMR (150 MHz, D₂O) δ (ppm) 172.44, 155.27, 151.78, 117.50, 114.14, 80.79, 75.08, 74.32, 61.23, 59.86, 54.64, 46.08, 21.58. HRMS (m/z): [M+H]⁺ calcd for C₁₅H₂₁NO₆S: 343.1168; found 344.1157.

Synthesis of Me-5SGlcNAc (16).

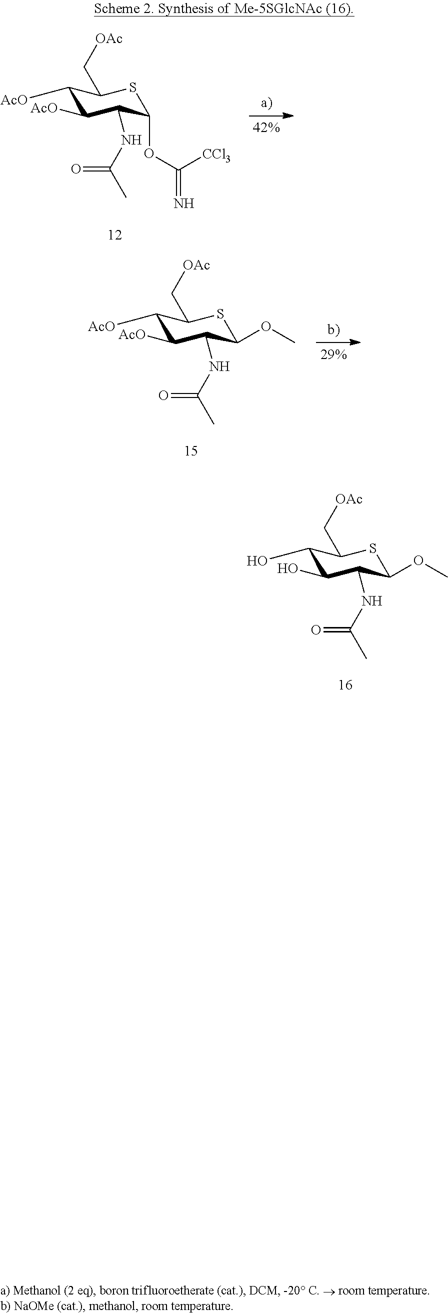

Methyl 2-acetamido-3,4,6-tri-O-acetyl-2-deoxy-5-thio-a-D-glucopyranoside (15). 12 (0.050 g, 0.11 mmol) was dissolved in dry dichloromethane (0.762 mL). Two equivalents of dry methanol (0.009 mL, 0.211 mmol) was added to the solution at -20° C. A catalytic amount of boron trifluoroethyletherate was added to the reaction mixture and allowed to warm to room temperature. Once the reaction was complete, two equivalents of triethylamine (0.03 mL) was added to the solution to quench the trifluoroethyletherate. The reaction was concentrated directly on a high vacuum rotary evaporator and run immediately through a silica gel column in a solvent system of ethyl acetate. The ratio of α to β anomers was found to be 1:1 by ¹1-1NMR, and the p anomer was slightly more polar than the α anomer by TLC. The final O-product was isolated through a second purification using flash column chromatography in ethyl acetate in order to separate the two anomers. 15 was isolated (0.0154 g, 42%) with an overall yield of a and 0 anomers of 0.0354 g, 96%. ¹H-NMR (600 MHz, MeOH, D₄) d (ppm) 5.22 (dd, J=9.74 Hz, 1H), 5.06 (dd, J=9.53 Hz, 1H), 4.64 (d, J=8.76 Hz, 1H), 4.40 (dd, J=11.86, 5.23 Hz, 1H), 4.26 (dd, J=9.21 Hz, 1H), 4.14 (dd, J=11.82, 3.87 Hz, 1H), 3.49 (s, 3H), 3.31 (m, 1H), 2.06 (s, 3H), 2.03 (s, 3H), 2.03 (s, 3H), 2.00 (s, 3H), 1.93 (s, 3H); ¹³C-NMR (150 MHz, MeOH, D₄) 8 (ppm) 171.93, 170.81, 170.12, 169.87, 82.96, 73.38, 71.96, 62.19, 57.76, 57.20, 40.46, 21.27, 19.19, 19.16, 19.09. HRMS (m/z): [M+H]⁺ calcd for C₁₅H₂₃NO₈S: 378.1223; found 378.1214.

Methyl 2-acetamido-2-deoxy-5-thio-a-D-glucopyranoside (16). 15 (0.0088 g, 0.023 mmol) was dissolved in methanol (0.35 mL) at room temperature. To this solution a catalytic amount of sodium methoxide was added (the pH of the reaction solution was 8-9). The reaction was followed closely by TLC and upon completion was neutralized (to pH 7) with a dilute solution of acetic acid in methanol (pH 4). The neutralized mixture was concentrated on the high vacuum rotary evaporator without heating. The reaction was purified using column chromatography in a solvent system of 15% methanol in chloroform. The reaction was crystallized in ethanol with ether. The final material was isolated as a white residue, which appeared solid (1.68 mg, 29%). ¹H-NMR (600 MHz, MeOH, D₄) δ (ppm) 4.31 (d, J=8.89 Hz, 1H), 3.90 (dd, J=9.26 Hz, 1H), 3.86 (dd, J=11.35, 3.93 Hz, 1H), 3.66 (dd, J=11.35, 6.59 Hz, 1H), 3.45 (dd, J=8.95 Hz, 11-1), 3.34 (s, 3H), 2.68 (m, 1H), 1.88 (s, 3H).¹³C-NMR (150 MHz, MeOH, D₄) δ (ppm) 172.32, 83.30, 75.44, 74.17, 61.68 to 59.50, 56.90, 46.03, 21.51. HRMS (m/z): [M+H]⁺ calcd for C₉H₁₇NO₅S: 252.0906; found 252.0913.

Synthesis of Ac-5SGlcNAz (41).

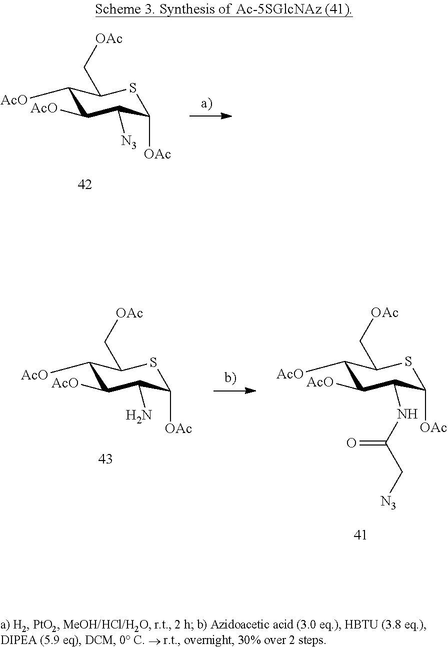

1,3,4, 6-Tetra-O-acetyl-2-azido-2-deoxy-5-thio-α-D-glucopyranoside (42) Synthesized from D-glucofurano-3,6-lactone using literature procedures with minor modifications in 13 steps⁵⁸⁻⁶⁰. Careful flash silica gel column chromatography using a gradient of hexanes:ethyl actetate in a ratio of 5:1 to 4:1 to 3:1, followed by preparative thin layer chromatography using a solvent system of hexanes:ethyl acetate in a ratio of 4:1 yielded pure α-anomer as a colourless syrup (for which the characterisation is described) and impure β-anomer which was not used further. ¹H-NMR (500 MHz, CDCl₃) δ (ppm) 6.10 (d, J=3.0 Hz, 1H), 5.40 (t, J=10.0 Hz, 1H), 5.32 (t, J=10.0 Hz, 1H), 4.39 (dd, J=12.0, 5.0 Hz, 1H), 4.03 (dd, J=12.0, 3.0 Hz, 1H), 3.88 (dd, J=10.0, 3.0 Hz, 1H), 3.56 (ddd, J=11.0, 5.0, 3.0 Hz, 1H), 2.19 (s, 3H), 2.11 (s, 3H), 2.07 (s, 3H), 2.05 (s, 3H); ¹³C-NMR (125 MHz, CDCl₃) δ (ppm) 170.45, 169.71, 169.51, 168.67, 71.74, 71.70, 64.88, 60.88, 39.80, 20.98, 20.60, 20.59, 20.50. HRMS (m/z): [M+NH₄]⁺ calcd for C₁₆H₂₆NO₁₀S: 424.1272; found 424.1267. [M+Na]⁺ calcd for C₁₆H₂₂NaO₁₀S: 429.0286; found 429.0280.

1,3, 4,6-Tetra-O-acetyl-2-azidoacetamido-2-deoxy-5-thio-a-D-glucopyranose (41) 2-Azido sugar 42 (42 mg, 0.11 mmol) and PtO₂ (22 mg, 0.097 mmol) were suspended in MeOH (5.0 mL) and 1 N HCl (aq., 1.0 mL) was added. The resulting reaction mixture was stirred under an atmosphere of H₂ (1 atm) at r.t. for 2 hours and then filtered through Celite. The filtrate was concentrated under reduced vacuum, the residue was co-evaporated with toluene (2×5.0 mL), and was then dried under high vacuum for 2 hours to afford crude intermediate 2-amino-1,3,4,6-tetra-O-acetyl-5-thio-alpha-a-D-glueopyranose hydrochloride (43) as a brown syrup (39 mg). This material was used in the next step directly without purification. To a solution of 43 (39 mg, 0.11 mmol) in dry CH₂Cl₂ (4.0 mL), was added HBTU (140 mg, 0.37 mmol), azidoacetic acid⁶¹ (30 mg, 0.30 mmol), and then DIPEA (0.10 mL, 0.58 mmol) was added drop-wise at 0° C. The resultant reaction mixture was stirred overnight before being quenched by addition of sat. NaHCO₃ (aq.) (10 mL), and the reaction mixture was extracted with CH₂Cl₂ (3×10 mL). The combined organic extracts were washed with brine (1×10 mL) and dried (MgSO₄). After filtration and concentration under reduced pressure, the residue was purified by flash column silica chromatography (hexanes:ethyl acetate, 3:1 to 2:1 then 1:1), followed by preparative TLC (CH₂Cl₂:MeOH, 20:1) to afford a pale yellow syrup (13 mg, 30%). ¹H-NMR (500 MHz, CDCl₃) (5 (ppm) 6.57 (brd, J=8.5 Hz, 1H, NH), 6.00 (d, J=2.5 Hz, 1H, H-1), 5.40 (t, J=10.0 Hz, 1H, H-4), 5.24 (t, J=10.0 Hz, 1H, H-3), 4.61 (m, 1H, H-2), 4.37 (dd, J=12.0, 5.0 Hz, 1H, H-6), 4.05 (dd, J=12.0, 3.0 Hz, 1H, H-6’), 3.92 (s, 2H, N₃CH₂), 3.51 (ddd, J=10.5, 5.0, 3.0 Hz, 1H, H-5), 2.21 (s, 3H), 2.08 (s, 3H), 2.06 (s, 3H), 2.05 (s, 3H); ¹³C-NMR (125 MHz, CDCl₃) δ (ppm) 171.43, 170.53, 169.17, 168.83, 168.51, 72.20 (C-1), 71.34 (C-4), 71.30 (C-3), 61.02 (C-6), 55.42 (C-2), 52.47 (N₃CH₂), 39.82 (C-5), 21.04, 20.63, 20.59, 20.52. HRMS (m/z): [M+H]⁺ calcd for C₁₆H₂₃N₄O₉S: 447.1180; found 447.1180. [M+NH₄]⁺ calcd for C ₁₆H₂₆N₅O₉S: 464.1446; found 464.1440. [M+Na]⁺ calcd for C₁₆H₂₂N₄NaO₉S: 469.1000; found 469.0999.

Characterization of UDP-5SGlcNAc: 5′-(2-acetamido-2-deoxy-5-thio-a-D-glucopyranosyl)diphosphate triethylammonium salt—The enzymatic synthesis was carried out as described in the methods section. Uridine ¹H NMR (600 MHz, D₂O): δ (ppm) 2.08 (s, 3H, NH(CO)CH₃), 3.38 (ddd, 1H, J_{5′,6a″}=5.4 Hz, J_{5′,6b″}=3.0 Hz, H-5″), 3.71 (dd, 1H, J_{4′,5″}=10.2 Hz, H-4″), 3.75 (dd, 1H, J_{3″,2″}=9.6 Hz, J_{3″,4″}=9.0 Hz, H-3″), 3.89 (dd, 1H, J_{6b′,6a″}=12.0 Hz, H-6G″), 3.97 (dd, 1H, H-6a″), 4.91 (m, 1H, H-2″), 4.20 (m, 1H, H-5b″), 4.25 (m, 1H, J_{5a′,5b′}=11.4 Hz, H-5a′), 4.29 (m, 1H, H-4′), ¹³C NMR (150 MHz, D₂O): δ (ppm) 22.06 (NH(CO)CH₃), 43.59 (C-5″), 57.74 (C-2″), 59.91 (C-6″), 64.89 (C-5′), 69.61 (C-3′), 72.03 (C-3″), 73.62 (C-4″), 73.75 (C-2′), 76.62 (C-1″), 83.17 (C-4′), 88.39 (C-1′), 102.63 (C-5), 141.63 (C-6), 151.78 (C-2), 166.20 (C-4), 169.13 (NH(CO)CH₃). ³¹P NMR (242 MHz, D₂O): (ppm) −10.66 (d, J_{p,p}=21.8 Hz), −12.09 (d). HRMS (m/z): [M−H]⁻ calcd for C₁₇H₂₆N₃O₁₆P₂S: 622.0519, found 622.0514. (FIGS. 1F-K).

### EXAMPLE III

### Biosynthesis and Characterization of UDP-5SGlcNAc

The end product of the hexosamine biosynthetic pathway (HBP) is UDP-GlcNAc, the donor substrate used by OGT, as depicted in FIG. 1B. The first and rate-limiting step in the de novo pathway, where fructose-6-phosphate (diverted from glycolysis) is converted to glutamine-6-phosphate, is catalyzed by glutamine: fructose-6-phosphate amidotransferase (GFAT). Glutamine-6-phosphate is transformed into GlcNAc-6-phosphate by acetyl-CoA:D-glucosamine-6-phosphate N-acetyltransferase (GAT). The salvage pathway recycles cellular GlcNAc, which is converted into GlcNAc-6-phosphate by GlcNAc kinase (GNK). GlcNAc-6-phosphate is converted into GlcNAc-1-phosphate by GlcNAc mutase (AGM), and then to the end product, UDP-GlcNAc, by UDP-GlcNAc pyrophosphorylase (AGX1). The 5-thio analogue is converted via the salvage pathway to generate intracellular UDP-5SGlcNAc. Ac-5SGlcNAc is deacetylated by cell esterases.

We evaluated whether 5SGlcNAc (FIG. 1C) could be transformed into UDP-5SGlcNAc by human HBP enzymes. 5SGlcNAc was synthesized as described herein. Using this material, UDP-5SGlcNAc (FIG. 1C) was prepared in a reaction containing 5SGlcNAc, ATP, UTP, and recombinant human GlcNAc kinase (GNK), GlcNAc mutase (AGM) and UDP-GlcNAc pyrophosphorylase (AGXI). The reaction, monitored by capillary electrophoresis (CE), showed the production of a UDP-sugar (FIG. 1D), which was purified and shown to be UDP-5SGlcNAc. Therefore, UDP-5SGlcNAc can be biosynthesized from 5SGlcNAc by the mammalian enzymes of the HBP.

### EXAMPLE IV

### Inhibition of OGT by UDP-5SGlcNAc

We then assayed the ability of OGT to use UDP-5SGlcNAc as a donor using nup62 as an acceptor^{52,53} and found it was an approximately 14-fold worse substrate as compared to UDP-GlcNAc. Since UDP-5SGlcNAc was found to be a relative poor substrate in this assay, we assessed its inhibitory activity toward OGT. Using radiolabelled UDP-GlcNAc as the donor, and nup62 as the acceptor, the amount of O-GlcNAc transferred was evaluated in the presence of increasing concentrations of UDP-5SGlcNAc. UDP-5SGlcNAc is an effective inhibitor of OGT with a K, value of 8 (FIG. 1E), indicating that it binds as well as the natural substrate UDP-GlcNAc (Kₘ=2-7 μM)^{40,53}. Therefore, UDP-5SGlcNAc is a poor substrate for OGT, and is a good inhibitor of OGT in vitro.

### EXAMPLE V

### Treatment of Cells with 5SGlcNAc

With these results in hand we evaluated the effect of treating cells with 5SGlcNAc. We treated cultured COS-7 cells for 24 hours with 5SGlcNAc as well as its peracetylated analogue (Ac-5SGlcNAc). Both compounds decreased O-GlcNAc levels in a dose-dependent fashion as evaluated by western blots using an O-GlcNAc-directed antibody (CTD110.6) (FIGS. 2A and 2G). Other commercial O-GlcNAc antibodies (RL2 and HGAC85) reveal similar decreases in O-GlcNAc levels (FIG. 2H). The western blots reveal the EC₅₀ values for Ac-5SGlcNAc and 5SGlcNAc to be 5 μM and 700 μM respectively, which is consistent with acetylation facilitating entry of 5SGlcNAc into cells. Hence in subsequent experiments we used Ac-5SGlcNAc.

We also observed a time-dependent decrease in O-GlcNAc levels, with almost no O-GlcNAc being detectable after 24 hours (FIG. 2B). Dosing for 5 days showed O-GlcNAc levels dropped in the first day and remained low (FIG. 2C). In these studies there were no apparent changes in cell morphology or rate of proliferation at the doses assayed. We observed Ac-5SGlcNAc administration induced a compensatory decrease in OGA levels and an increase in OGT levels (FIG. 2D). Despite OGA levels continuing to drop and OGT levels rising over 5 days of dosing, O-GlcNAc levels remained low and unchanged after 24 hours (FIG. 2C).

We next investigated the reversibility of the effect of Ac-5SGlcNAc treatment and found that following a change of media containing no compound, O-GlcNAc returned to basal levels within 24 hours (FIG. 24 A time course and dose response study in CHO cells (FIGS. 2J-K), revealed a similar EC₅₀ value (0.8 μM). Various other cell lines showed equivalent responses (FIG. 2L).

### EXAMPLE VI

### O-GlcNAc Levels on nup62 After Treatment with Ac-5SGlcNAc

We next probed O-GlcNAc levels on an individual protein after treatment with Ac-5SGlcNAc by immunoprecipitating nup62 from cell lysates. To probe for O-GlcNAc on immunoprecipitated nup62 we used a commercial chemoenzymatic method⁵⁴ that involves chemoselective Staudinger ligation⁵⁵ of biotin to terminal GlcNAc residues. Labeled and unlabeled nup62 from cell lysates were probed with streptavidin (FIG. 2E); nup62 from untreated cells shows clear O-GlcNAc modification, whereas nup62 derived from cells treated with Ac-5SGlcNAc shows almost no modification. Previous studies revealed that nup62 can be observed as two bands under certain SDS-PAGE conditions; the upper band corresponds to O-GlcNAc modified nup62 whereas the lower band corresponds to deglycosylated nup62⁹′⁵². We therefore analyzed immunoprecipitated nup62 in this way and found that untreated cells yielded only the upper band in western blots and this species was heavily O-GlcNAc modified (FIG. 2F). Treating this nup62 precipitate with a bacterial homologue of OGA (BtGH84⁴³) greatly diminished O-GlcNAc immunoreactivity and the resulting deglycosylated nup62 appeared as a lower band. nup62 from cells treated with Ac-5SGlcNAc, however, appeared as a doublet of bands consistent with there being a mixture of two species; an upper band, which is slightly O-GlcNAc modified, and a lower band, which is an essentially unmodified species of nup62. BtGH84 digestion of nup62 derived from Ac-5SGlcNAc treated cells resulted in near complete loss of remaining O-GlcNAc and only the lower band remained, consistent with remaining O-GlcNAc on the partly modified upper band of nup62 being removed. These results collectively show that 5SGlcNAc dramatically decreases O-GlcNAc levels even on proteins that are constitutively and heavily O-GlcNAc modified in cells.

### EXAMPLE VII

### Immunoreactivity of GlcNAc and 5SGlcNAc

We tested the immunoreactivity of GlcNAc and 5SGlcNAc with CTD 110.6 through blocking experiments using different concentrations of methyl 2-acetamido-2-deoxy-P-D-glucopyranoside (Me-GlcNAc) and methyl 2-acetamido-2-deoxy-5-thio-β-D-glucopyranoside (Me-5SGlcNAc; FIGS. 1C and 4A). We find that Me-5SGlcNAc blocks binding almost as well as Me-GlcNAc, supporting the view that 5SGlcNAc does not accumulate to a significant extent on proteins. Nevertheless, we assayed the ability of OGA to process 5SGlcNAc by synthesizing para-methoxyphenyl 5SGlcNAc (pMP-5SGlcNAc, FIG. 1C) and evaluating the OGA-catalyzed hydrolysis of this compound compared to para-methoxyphenyl-GlcNAc⁵⁶ (pMP-GlcNAc). Michaelis-Menten kinetics (FIGS. 4B-C) reveal human OGA processes pMP-5SGlcNAc (Vₘₐₓ/E₀K_{M}=8.1 μmol s⁻¹ mg⁻¹ M⁻¹) only 5-fold less efficiently than pMP-GlcNAc (Vₘₐₓ/E₀K_{M}=40.7 μmol s⁻¹ mg⁻¹ M⁻¹).

An alternate process that could cause decreased O-GlcNAc levels in cells treated with Ac-5SGlcNAc is that the pool of UDP-GlcNAc in cells be greatly diminished. To verify that UDP-5SGlcNAc is being biosynthesized within cells, and to evaluate the cellular levels of UDP-GlcNAc and other nucleotide sugar donors, we treated COS-7 cells with different concentrations of Ac-5SGlcNAc. Analysis of the extracted UDP-sugars using CE (FIGS. 3A-B, F-G) revealed a dose dependent increase in the amount of UDP-5SGlcNAc in cells and a small decrease in UDP-GlcNAc levels. An additional peak corresponding to a synthetic standard of UDP-5SGalNAc is also observed (FIGS. 3E-F), while uridine diphosphoglucose and uridine diphosphogalactose levels are unaffected. The marked accumulation of UDP-5SGlcNAc is consistent with OGT being unable to use it as a substrate. At 50 μM we find near complete loss of O-GlcNAc in the cell lines tested, yet UDP-GlcNAc levels are still more than 60% of basal levels; a decrease that is unlikely to account for the low O-GlcNAc levels observed in treated cells. Therefore, 5SGlcNAc can efficiently traverse the HBP to form levels of UDP-5SGlcNAc that proficiently inhibit OGT in cells.

### EXAMPLE VIII

### Cell Surface Glycosylation

To assess whether cell surface glycosylation could be affected through changes in UDP-GlcNAc availability or inhibition of GlcNAc transferases within the secretory pathway, we treated COS-7 cells with a range of Ac-5SGlcNAc concentrations and evaluated the effect on various glycans using lectin blots. All lectins tested showed no change in glycosylation, even at the high doses of Ac-5SGlcNAc assayed (FIGS. 3C and H). These lectins probed for both high mannose (ConA and GNA) and complex N-glycans (PHA-L, SNA and MAA). We also examined the effect of Ac-5SGlcNAc on N-glycosylation of an individual protein. to Immunoprecipitated IgG produced by a mouse hybridoma cell line appeared to possess only high mannose glycans. There appeared to be a slight decrease in GNA and ConA reactivity when using a very high dose of Ac-5SGlcNAc (FIGS. 3D and I). These collective observations are consistent with reports showing that EMEG32^{-/-} cells, which are deficient in the biosynthesis of GlcNAc, have very low UDP-GlcNAc levels but N-glycosylation is virtually unaffected⁵⁷. Apparently, ˜5% of the normal UDP-GlcNAc levels found in cells is sufficient for N-glycosylation⁵⁷, a level much lower than that observed when using inhibitory concentrations of 5SGlcNAc to block OGT.

### EXAMPLE IX

### Cellular Responses to Ac-5SGlcNAc

Cellular responses to Ac-5SGlcNAc (9) treatment and consequent decreases in O-GlcNAc levels were evaluated. In the studies and cell lines examined, there were qualitatively no apparent changes in cell morphology or rate of proliferation at the doses assayed. We quantitatively evaluated cell proliferation and established growth curves over the course of 5 days in both CHO and EMEG32 heterozygous mouse embryonic fibroblast cells treated with 50 μM Ac-5SGlcNAc (9), 50 μM Ac-GlcNAc (G), or vehicle (FIGS. 5A-B). For CHO cells the growth rate of Ac-5SGlcNAc (9) treated cells was indistinguishable from those of Ac-GlcNAc (G) and vehicle treated cells. In EMEG32 heterozygotes, the growth rates of the Ac-5SGlcNAc (9) and Ac-GlcNAc (G) treated cells were indistinguishable but were both lower than vehicle treated cells. Interestingly, despite the absence of Ac-5SGlcNAc (9) specific effects on growth rate, decreases were observed in the levels of transcription factor Sp1 (FIGS. 5C-D).

### EXAMPLE X

### Metabolic Feeding of 5SGlcNAz

To probe the possibility that 5SGlcNAc is being incorporated onto proteins, we used a metabolic feeding and chemoselective ligation strategy using 2-azidoacetamido-2-deoxy-D-glucopyranose (GlcNAz), which can be assimilated by the HBP to form uridine diphospho-N-azidoacetylglucosamine (UDP-GlcNAz) and then be installed onto proteins as O-GlcNAz⁹. Using a chemoselective ligation to install a reporter group allows sensitive detection of O-GlcNAz modified proteins⁹. Accordingly, we evaluated whether 2-azidoacetamido-2-deoxy-5-thio-D-glucopyranose (5SGlcNAz) is incorporated onto proteins to determine whether 5SGlcNAc might be transferred onto proteins in cells. This approach has the advantage that it relies on neither recognition of potential O-5SGlcNAc by the three antibodies tested nor by the GalT used for the chemoenzymatic labelling approach. We therefore carried out the synthesis of 1,3,4,6-tetra-O-acetyl-2-azidoacetamido-2-deoxy-5-thio-α-D-glucopyranose (Ac-5SGlcNAz, 41, FIG. 6A) in 15 steps, the last two steps of which diverge from a previously known compound⁶⁰ (see Scheme 3, and FIG. 6D-E for details of the synthesis and characterization). Briefly, these two steps involved catalytic hydrogenation followed by DCC coupling, and furnished Ac-5SGlcNAz (41) in reasonable yield. Treating cells with either vehicle, 1,3,4,6-tetra-O-acetyl-2-azidoacetamido-2-deoxy-ot-D-glucopyranose⁹ (Ac-GlcNAz) or Ac-5SGlcNAz (41) revealed that only 5SGlcNAz decreased O-GlcNAc levels. Proteins from cells treated with vehicle, Ac-GlcNAz , or Ac-5SGlcNAz (41) were collected and subjected to Staudinger ligation⁵⁵ with biotin phosphine (FIG. 6A). Following the ligation, proteins were blotted onto nitrocellulose and probed using streptavidin-horse radish peroxidase. Only proteins from cells treated with Ac-GlcNAz revealed any signal, whereas the signal from cells treated with vehicle were indistinguishable from those treated with Ac-5SGlcNAz (41) (FIG. 6B). Similarly, we immunoprecipitated nup62 from cells treated with vehicle, Ac-GlcNAz, or Ac-5SGlcNAz (41), and prior to Staudinger ligation, incubated half of each sample with BtGH84⁴³. The resulting blot, probed with streptavidin-HRP, once again only showed signal from nup62 immunoprecipitated from cells treated with Ac-GlcNAz, and only in the sample that had not been subjected to BtGH84 hydrolysis (FIG. 6C). The observation that Ac-5SGlcNAz (41) decreases O-GlcNAc levels indicates that uridine diphospho-5-thio-N-azidoacetylglucosamine (UDP-5SGlcNAz) is formed within cells where it acts to inhibit OGT. The chemoselective ligation data, however, indicates that 5SGlcNAz does not accumulate on proteins to any measurable level. These data indicated that 5SGlcNAc does not accumulate on proteins, and that decreased O-GlcNAc levels arise from inhibition of OGT by UDP-5SGlcNAc.

### EXAMPLE XI

### Synthesis of Ac-5SGlcNAc Analogues

A total of 27 analogues, as shown below, were synthesized based on to the Ac-5SGlcNAc (9) template. These analogues incorporate different alkyl substituents at the C2 position, and were synthesized in a protected (acetylated at Cl, C3, C4 and C6) and deprotected (free hydroxyl) form.

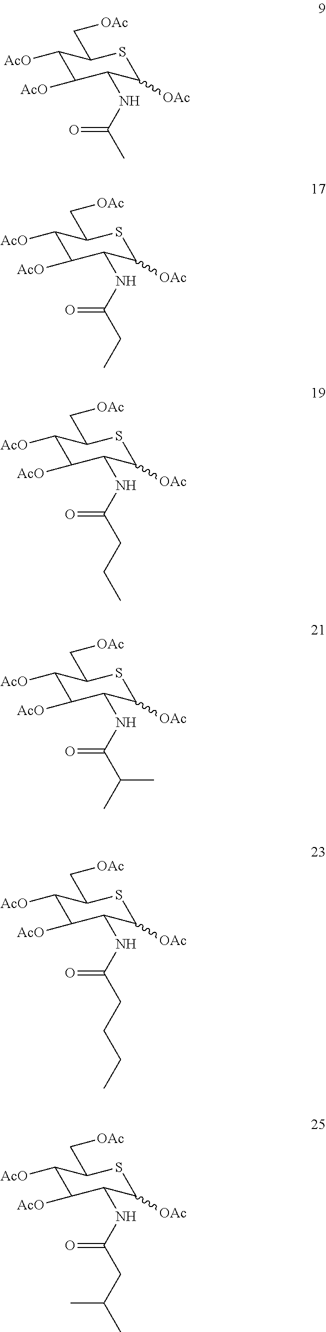

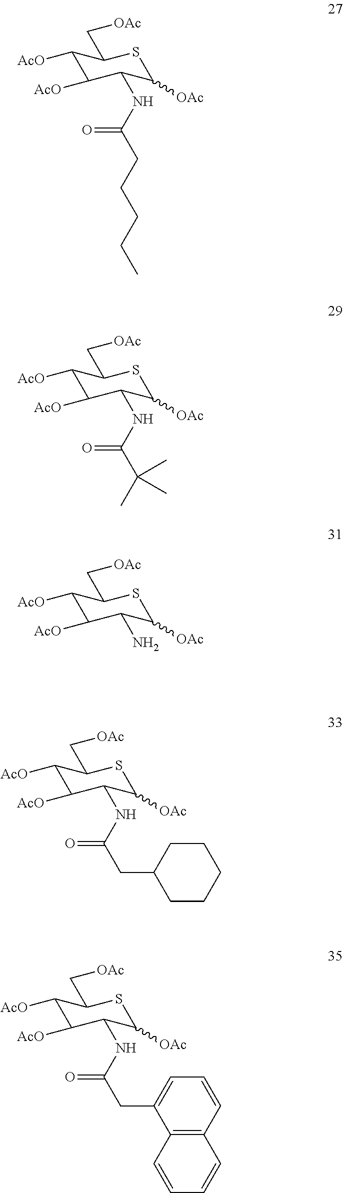 [SMILES: CCCCCC(=O)NC1C(OC(C)=O)SC(COC(C)=O)C(OC(C)=O)C1OC(C)=O ; CC(=O)OCC1SC(OC(C)=O)C(NC(=O)C(C)(C)C)C(OC(C)=O)C1OC(C)=O ; CC(=O)OCC1SC(OC(C)=O)C(N)C(OC(C)=O)C1OC(C)=O ; CC(=O)OCC1SC(OC(C)=O)C(NC(=O)CC2CCCCC2)C(OC(C)=O)C1OC(C)=O ; CC(=O)OCC1SC(OC(C)=O)C(NC(=O)Cc2cccc3ccccc23)C(OC(C)=O)C1OC(C)=O]

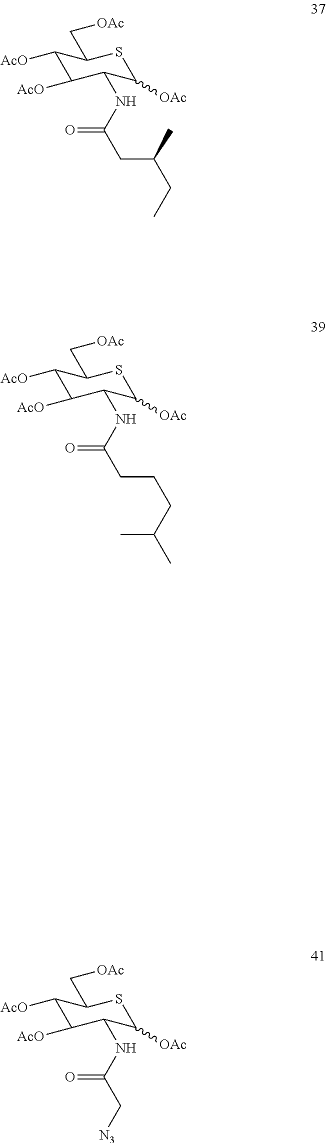 [SMILES: CC[C@H](C)CC(=O)NC1C(OC(C)=O)SC(COC(C)=O)C(OC(C)=O)C1OC(C)=O ; CC(=O)OCC1SC(OC(C)=O)C(NC(=O)CCCC(C)C)C(OC(C)=O)C1OC(C)=O ; CC(=O)OCC1SC(OC(C)=O)C(NC(=O)CN=[N+]=[N-])C(OC(C)=O)C1OC(C)=O]

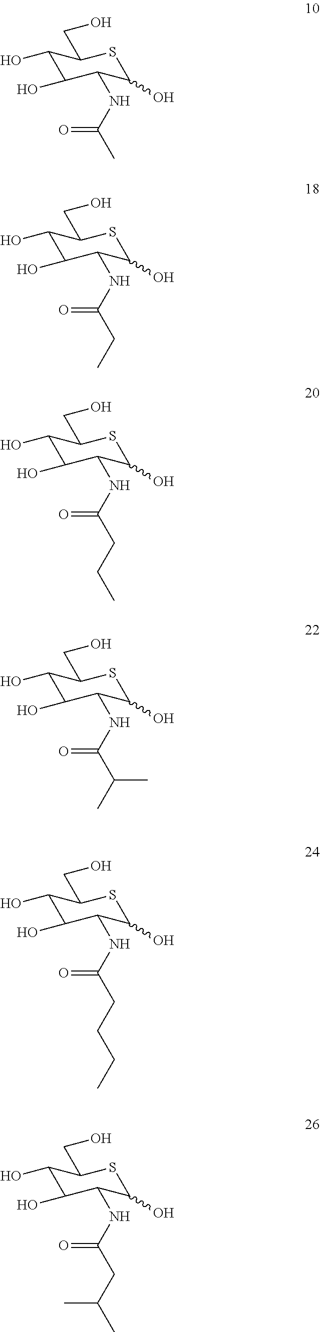

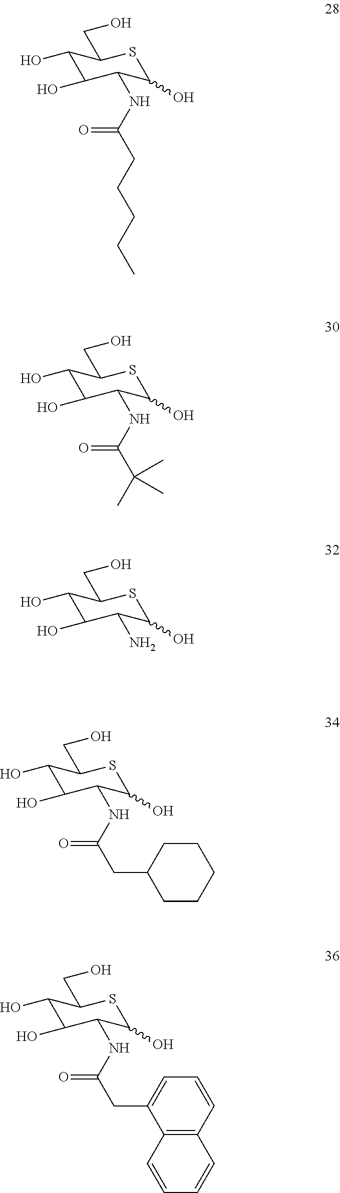 [SMILES: CCCCCC(=O)NC1C(O)SC(CO)C(O)C1O ; CC(C)(C)C(=O)NC1C(O)SC(CO)C(O)C1O ; NC1C(O)SC(CO)C(O)C1O ; O=C(CC1CCCCC1)NC1C(O)SC(CO)C(O)C1O ; O=C(Cc1cccc2ccccc12)NC1C(O)SC(CO)C(O)C1O]

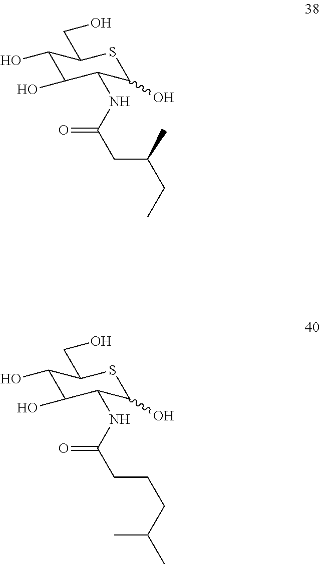 [SMILES: CC[C@H](C)CC(=O)NC1C(O)SC(CO)C(O)C1O ; CC(C)CCCC(=O)NC1C(O)SC(CO)C(O)C1O]

In general, the compounds were prepared according to the synthetic route outlined in Scheme 4.

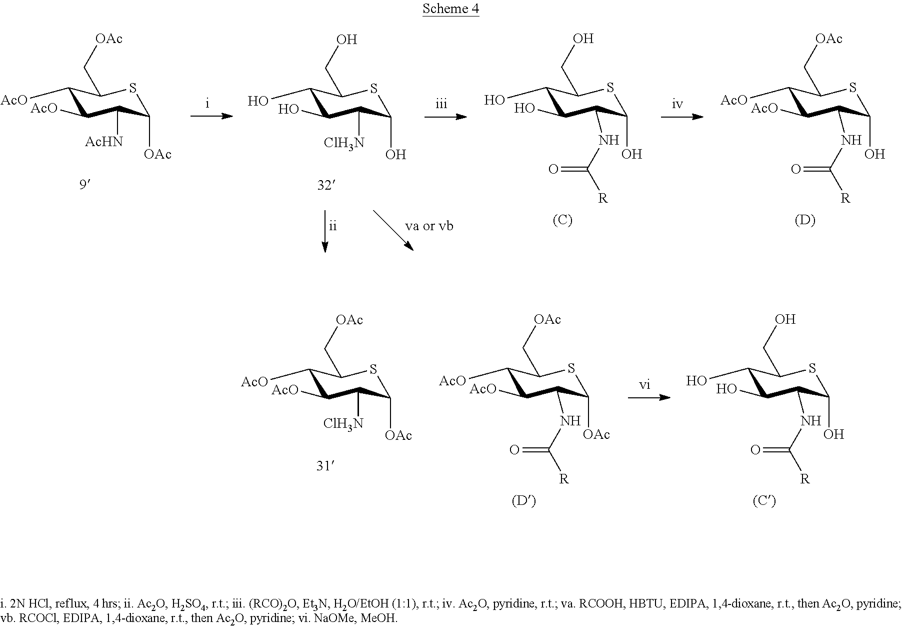

General Procedure A: Synthesis of N-acyl-α-D-5-thio-glucosamine (C) using acid anhydrides. Acid anhydrides (1.5 eq.) and Et₃N (1.1 eq.) were added into the solution of 5-thio-D-glucosamine hydrochloride (32′) in solvent H₂OEtOH (1:1), the resultant reaction mixture was stirred at r.t. for 18-20 hrs. The solvent was then removed in vacuum, and the desired materials were isolated as solids by flash silica chromatography using a solvent system of EtOAc:MeOH:H₂O in ratios ranging from 15:1:0.5 to 10:1:0.5 as appropriate.

General Procedure B: Synthesis of N-acyl-1,3,4,6-tetra-O-acetyl-α-D-5-thio-glucosamine (D). N-acyl-a-D-5-thio-glucosamine (C) was dissolved in pyridine and Ac₂O (10 eq.) was added at r.t. The resultant reaction mixture was stirred at r.t. for 18-20 hrs. The solvent was then removed in vacuum, and the desired materials were isolated as solids by flash silica chromatography using a solvent system of Hex:EtOAc in ratios ranging from 3:1 to 1:1 as appropriate.

General Procedure C: Synthesis of N-acyl-1,3,4,6-tetra-O-acetyl-α-D-5-thio-glucosamine (D′) using acids. To the suspension of 5-thio-D-glucosamine hydrochloride (32′) in 1,4-dixoane was added acids (1.5-1.8 eq.), EDIPA (2.0 eq.) and HBTU (2.0 eq.), the resultant reaction mixture was stirred at r.t. for 18-20 hrs. The solvent was then removed in vacuum, and the N-acylated intermediates were isolated as solids by flash silica chromatography using a solvent system of EtOAc:MeOH:H₂O in ratios ranging from 15:1:0.5 to 10:1:0.5 as appropriate. Then the intermediates were dissolved in pyridine, and Ac₂O (10 eq.) was added at r.t. The resultant reaction mixture was stirred at r.t. for 18-20 hrs. The solvent was then removed in vacuum, and the desired materials were isolated as solids by flash silica chromatography using a solvent system of Hex:EtOAc in ratios ranging from 3:1 to 1:1 as appropriate.

General Procedure D: Synthesis of N-acyl-1,3,4,6-tetra-O-acetyl-α-D-5-thio-glucosamine (D′) using acid chloride. To the suspension of 5-thio-D-glucosamine hydrochloride (32′) in 1,4-dixoane was added acids (1.5-2.0 eq.) and EDIPA (1.5-2.0 eq.), the resultant reaction mixture was stirred at r.t. for 18-20 hrs. The solvent was then removed in vacuum, and the N-acylated intermediates were isolated as solids by flash silica chromatography using a solvent system of EtOAc:MeOH:H₂O in ratios ranging from 15:1:0.5 to 10:1:0.5 as appropriate. Then the intermediates were dissolved in pyridine, and Ac₂O (10 eq.) was added at r.t. The resultant reaction mixture was stirred at r.t. for 18-20 hrs. The solvent was then removed in vacuum, and the desired materials were isolated as solids by flash silica chromatography using a solvent system of Hex:EtOAc in ratios ranging from 3:1 to 1:1 as appropriate.

General Procedure E: Synthesis of N-acyl-α-D-5-thio-glucosamine (C′). Sodium methoxide was added into the solution of N-acyl-1,3,4,6-tetra-O-acetyl-α-D-5-thio-glucosamine (D′) in MeOH to keep pH-10, and the resultant reaction mixture was stirred at r.t. for 2 hrs. The reaction was then neutralized with Amberlite IR 120 (H⁺) resin. After filtration and removal of the solvent, the desired materials were isolated as solids by flash silica chromatography using a solvent system of to EtOAc:MeOH:H₂O in ratios ranging from 15:1:0.5 to 10:1:0.5 as appropriate.

### Compound 9 and 10: N-Acetyl-1,3,4,6-tetra-O-acetyl-α-D-5-thio-glucosamine (9) and N-acetyl-α-D-5-thio-glucosamine (10)

The title compound 9 and 10 was synthesized from N-acetyl-D-glucosamine in 8 and 9 steps respectively (ref: Nature Chem. Biol. 2011, 7, 174-181.).

### Compound 32′: α-D-5-thio-glucosamine hydrochloride (32′)

Compound N-acetyl-1,3,4,6-tetra-O-acetyl-α-D-5-thio-glucosamine (1, 945 mg, 2.33 mmol) was suspended in 2 N HCl (aq., 15 mL), and the resultant reaction mixture was stirred under reflux for 4 hrs. After cooling down to r.t., the reaction mixture was concentrated under high vacuum, and was co-evaporated with toluene (8×15 mL). The residue was then re-dissolved in MeOH (100 mL), and activated carbon was added to the solution. After stirring for 30 mins, the mixture was filtered through Celite, and concentrated to dryness. The residue was dried under high vacuum to afford 32′ as a solid (520 mg, 96%), which was used directly to the next reaction without purification.¹H-NMR (500 MHz, D₂O): δ 5.23 (d, J=3.00 Hz, 1H), 3.94 (dd, J=12.0, 5.50 Hz, 1H), 3.89 (dd, J=12.0, 3.00 Hz, 1H), 3.84 (dd, J=10.0, 9.00 Hz, 1H), 3.67 (dd, J=10.5, 9.00 Hz, 1H), 3.61 (dd, J=10.5, 3.00 Hz, 1H), 3.30 (ddd, J=10.0, 5.50, 3.00 Hz, 1H). ¹³C-NMR (125 MHz, D₂O): δ 73.98, 71.16, 70.51, 60.45, 59.06, 43.84. HRMS (m/z): C₆H₁₄NO₄S (M+H)⁺: Calcd, 196.0638; Found, 196.0634. C₆H₁₃NNaO₄S (M+Na)⁺: Calcd, 218.0457; Found, 218.0457. C₆H₁₃KNO₄S (M+K)⁺; Calcd, 234.0197; Found, 234.0201.

### Compound 31′: 1,3,4,6-tetra-O-acetyl-a-D-5-thio-glucosamine sulphuric acid salt (31′)

a-D-5-thio-Glucosamine hydrochloride 9 (20.0 mg, 0.086 mmol) was dissolved in Ac₂O (0.30 mL), and con. H₂SO₄ (1 drop, about 0.025 mL) was added, the resulting mixture was stirred at r.t. for 40 hrs. After addition of MeOH (2 drops), the mixture became clear. To the resultant reaction mixture was added EtOAc (about 3.0 mL) under ice-bath (0° C.) and stirred for several minutes. White solid was precipitated. Filtration and washing with EtOAc (20 mL) afford 31′ as a white solid, which was purified by recrystallization from EtOAcMeOH system. The yield was 10 mg (25%). ¹H-NMR (400 MHz, MeOD): δ 6.12 (d, J=3.20 Hz, 1H), 5.40 (t, J=10.0 Hz, 1H), 5.31 (t, J=10.0 Hz, 1H), 4.46 (dd, J=12.4, 4.80 Hz, 1H), 4.12 (dd, J=10.8, 3.20 Hz, 1), 4.04 (dd, J=12.4, 3.20 Hz, 1H), 3.72 (ddd, J=10.8, 4.40, 3.20 Hz, 1H), 2.23 (s, 3 14), 2.12 (s, 3H), 2.05 (s, 3H), 2.02 (s, 3H). ¹³C-NMR (100 M Hz, MeOD): δ 172.01, 171.58, 171.16, 170.17, 73.03, 72.10, 71.96, 62.01, 56.88, 40.84, 20.82, 20.77, 20.51, 20.48. HRMS (m/z): C₁₄H₂₂NO₈S (M+H)⁺: Calcd, 364.1061; Found, 364.1068. C₁₄H₂₁NNaO₈S (M+Na)⁺: Calcd, 386.0880; Found, 386.0890.

### Compound 18 and 17: N-Proprionyl-α-D-5-thio-glucosamine (18) and N-proprionyl-1,3,4,6-tetra-O-acetyl-α-D-5-thio-glucosamine (17)

The title compounds were prepared from the material thus obtained following General Procedures A and B. For 18: ¹H-NMR (400 MHz, D₂O): δ 4.99 (d, J=2.80 Hz, 1H, H-1), 4.11 (dd, J=10.4, 2.80 Hz, 1H, H-2), 3.95 (dd, J=12.0, 5.60 Hz, 1H, H-6a), 3.89 (dd, J=12.0, 3.20 Hz, 1H, H-6b), 3.73 (t, J=9.20 Hz, 1H, H-3), 3.68 (t, J=9.20 Hz, 1H, H-4), 3.28 (ddd, J=9.60, 5.60, 3.60 Hz, 1H, H-5), 2.31 (q, J=7.60 Hz, 2H, H-2’), 1.13 (t, J=7.60 Hz, 3H, H-3′). ¹³C-NMR (100 MHz, D₂O): δ 178.00 (C-1′), 74.04 (C-4), 71.75 (C-1), 71.52 (C-3), 60.20 (C-6), 58.05 (C-2), 43.15 (C-5), 29.08 (C-2′), 9.49 (C-3′). HRMS (m/z): C₉H₁₈NO₅S (M+H)⁺: Calcd, 252.0900; Found, 252.0895. C₉H₁₇NNaO₅S (M+Na)⁺: Calcd, 274.0720; Found, 274.0718. C₉H₁₇KNO₅S (M+K)⁺: Calcd, 290.0459; Found, 290.0454.

For 17: ¹H-NMR (500 MHz, CDCl₃): δ 5.93 (d, J=3.00 Hz, 1H), 5.71 (d, J=9.00 Hz, 1H), 5.37 (t, J=10.0 Hz, 1H), 5.18 (t, J=10.5 Hz, 1H), 4.66-4.61 (m, 1H), 4.32 (dd, J=12.0, 5.00 Hz), 4.03 (dd, J=12.0, 3.00 Hz), 3.46 (ddd, J=11.0, 5.00, 3.50 Hz, 1H), 2.17 (s, 3H), 2.15-2.05 (m, 2H), 2.06 (s, 3H), 2.03 (s, 3H), 2.01 (s, 3H), 1.06 (t, J=7.50 Hz, 3H). ¹³C-NMR (125 MHz, CDCl₃): δ 173.21, 171.57, 170.51, 169.08, 168.68, 72.69, 71.69, 71.43, 61.07, 54.99, 39.70, 29.50, 21.03, 20.58, 20.46, 9.63. HRMS (m/z): C₁₇H₂₆NO₉S (M+H)⁺: Calcd, 420.1323; Found, 420.1324. C₁₇H₂₉N₂O₉S (M+NH₄)⁺: Calcd, 437.1588; Found, 437.1591. C₁₇H₂₅NNaO₉S (M+Na)⁺: Calcd, 442.1142; Found, 442.1143. C₁₇H₂₅KNO₅S (M+K)⁺: Calcd, 458.0882; Found, 458.0883.

### Compound 20 and 19: N-(n-Butyryl)-a-D-5-thio-glucosamine (20) and N-(n-butyryl)-1,3,4,6-tetra-O-acetyl-a-D-5-thio-glucosamine (19)

The title compounds were prepared from the material thus obtained following General Procedures A and B. For 20: ¹H-NMR (500 MHz, D₂O): δ 4.97 (d, J=3.00 Hz, 1H=), 4.10 (dd, J=10.5, 3.00 Hz, 1H), 3.93 (dd, J=12.0, 5.50 Hz, 1 H), 3.89 (dd, J=12.0, 3.50 Hz, 1H), 3.70 (t, J=9.50 Hz, 1H), 3.66 (t, J=9.50 Hz, 1H), 3.27 (ddd, J=10.0, 5.50, 3.00 Hz, 1H), 2.26 (t, J=7.50 Hz, 2H), 1.65-1.57 (m, 2H), 0.90 (t, J=7.50 Hz, 3H). ¹³C-NMR (125 MHz, D₂O): δ 177.07, 74.02, 71.79, 71.41, 60.15, 58.02, 43.11, 37.56, 18.99, 12.63. HRMS (m/z): C₁₀H₂₀NO₅S (M-41)⁺: Calcd, 266.1057; Found, 266.1052. C₁₀H₁₉NNaO₅S (M+Na)⁺: Calcd, 288.0876; Found, 288.0876. _{C10H19KNO5S} (M+K)⁺: Calcd, 304.0616; Found, 304.0614.

For 19: ¹H-NMR (500 MHz, CDCl₃): δ 5.94 (d, J=3.00 Hz, 1H), 5.71 (d, J=9.00 Hz, 1H), 5.37 (t, J=10.0 Hz, 1H), 5.18 (t, J=10.0 Hz, 1H), 4.66-4.61 (m, 1H), 4.32 (dd, J=12.5, 5.00 Hz, 1H), 4.03 (dd, J=12.0, 3.00 Hz, 1H), 3.47 (ddd, J=11.0, 5.00, 3.50 Hz, 1H), 2.17 (s, 3H), 2.10-2.06 (m, 2H), 2.06 (s, 3 H), 2.03 (s, 3H), 2.02 (s, 3H), 1.60-1.53 (m, 2H), 0.88 (t, J=7.50 Hz, 3H. ¹³C-NMR (125 MHz, CDCl₃): δ 172.39, 171.63, 170.51, 169.11, 168.66, 72.69, 71.64, 71.46, 61.08, 55.01, 39.70, 38.71, 21.04, 20.64, 20.60, 20.49, 18.94, 13.51. HRMS (m/z): C₁₈H₂₈NO₉S (M+H)⁺: Calcd, 434.1479; Found, 434.1484. C₁₈H_{3I}N₂O₉S (M+NH₄)⁺: Calcd, 451.1745; Found, 451.1752. C₁₈H₂₇NNaO₉S (M+Na)⁺: Calcd, 456.1299; Found, 456.1304. C₁₈H₂₇KNO₅S (M+K)⁺: Calcd, 472.1038; Found, 472.1044.

### Compound 22 and 21: N-(i-Butyryl)-a-D-5-thio-glucosamine (22) and N-(i-butyryl)-1,3,4,6-tetra-O-acetyl-a-D-5-thio-glucosamine (21)

The title compounds were prepared from the material thus obtained following General Procedures A and B. For 22: ¹H-NMR (400 MHz, D₂O): δ 4.98 (d, J=2.80 Hz, 1H), 4.12 (dd, J=10.4, 2.80 Hz, 1H), 3.94 (dd, J=11.6, 5.60 Hz, 1H), 3.90 (dd, J=12.0, 3.20 Hz, 1H), 3.73 (t, J=9.60 Hz, 1H), 3.68 (t, J=9.60 Hz, 1H), 3.29 (ddd, J=9.60, 5.60, 3.20 Hz, 1H), 2.63-2.52 (m, 1H), 1.12 (d, J=6.80 Hz, 6 H). ¹³C-NMR (100 MHz, D₂O): δ 181.11, 74.07, 71.80, 71.48, 60.22, 57.93, 43.17, 34.97, 18.72, 18.55. HRMS (m/z): C₁₀H₂₀NO₅S (M+H)⁺: Calcd, 266.1057; Found, 266.1052. C₁₀H₁₉NNaO₅S (M+Na)⁺: Calcd, 288.0876; Found, 288.0874. C₁₀H₁₉KNO₅S (M+K)⁺: Calcd, 304.0616; Found, 304.0612.

For 21: ¹H-NMR (500 MHz, CDCl₃): δ 5.95 (d, J=3.00 Hz, 1H), 5.69 (d, J=9.00 Hz, 1H), 5.38 (t, J=10.0 Hz, 1H), 5.18 (t, J=10.0 Hz, 1H), 4.65-4.59 (m, 1H), 4.34 (dd, J=12.0, 5.00 Hz, 1H), 4.04 (dd, J=12.0, 3.00 Hz, 1H), 3.48 (ddd, J=11.0, 5.00, 3.50 Hz, 1H), 2.30-2.21 (m, 1H), 2.18 (s, 3H), 2.06 (s, 3H), 2.04 (s, 3H), 2.02 (s, 3H), 1.06 (d, J=5.00 Hz, 3H), 1.05 (d, J=5.00 Hz, 3H). ¹³C-NMR (125 MHz, CDCl₃): δ 176.32, 171.63, 170.54, 169.12, 168.65, 72.61, 71.66, 71.44, 61.10, 54.93, 39.73, 35.41, 21.00, 20.62, 20.49, 19.38, 19.17. HRMS (m/z): C₁₈H₂₈NO₉S (M+H)⁺: Calcd, 434.1479; Found, 434.1482. C₁₈H₃₁N₂O₉S (M+NH₄)⁺: Calcd, 451.1745; Found, 451.1743. C₁₈H₂₇NNaO₉S (M+Na)⁺: Calcd, 456.1299; Found, 456.1300. C₁₈H₂₇KNO₅S (M+K)⁺: Calcd, 472.1038; Found, 472.1038.

### Compound 24 and 23: N-(n-Valeryl)-α-D-5-thio-glucosamine (24) and N-(n-valeryl)-1,3,4, 6-tetra-O-acetyl-α-D-5-thio-glucosamine (23)

The title compounds were prepared from the material thus obtained following General Procedures A and B. For 24: ¹H-NMR (500 MHz, D₂O): δ 4.96 (d, J=2.50 Hz, 1H), 4.10 (dd, J=10.0, 3.00 Hz, 1H), 3.93 (dd, J=12.0, 5.50 Hz, 1H), 3.87 (dd, J=12.0, 3.00 Hz, 1H), 3.70 (t, J=9.50 Hz, 1H), 3.66 (t, J=10.0 Hz, 1H), 3.27 (ddd, J=9.50, 5.50, 3.00 Hz, 1H), 2.29 (t, J=7.50 Hz, 2H), 1.60-1.54 (m, 2H), 1.34-1.27 (m, 2H), 0.88 (t, J=7.50 Hz, 3H). ¹³C-NMR (125 MHz, D₂O): δ 177.19, 73.88, 71.66, 71.30, 60.00, 57.93, 43.00, 35.34, 27.47, 21.40, 12.90. HRMS (m/z): C₁₁H₂₂NO₅S (M+H)⁺: Calcd, 280.1213; Found, 280.1200. C_{t 1}1-1₂₁NNaO₅S (M+Na)⁺: Calcd, 302.1033; Found, 302.1023. C₁₁H₂₁KNO₅S (M+K)⁺: Calcd, 318.0772; Found, 318.0767.

For 23: ¹H-NMR (500 MHz, CDCl₃): δ 5.93 (d, J=3.50 Hz, 1H), 5.71 (d, J=8.50 Hz, 1H), 5.38 (dd, J=11.0, 10.0 Hz, 1H), 5.18 (dd, J=11.0, 10.0 Hz, 1H), 4.65-4.60 (m, 1H), 4.34 (dd, J=12.0, 5.00 Hz, 1H), 4.02 (dd, J=12.0, 3.50 Hz, 1H), 3.47 (ddd, J=10.5, 4.50, 3.00 Hz, 1H), 2.17 (s, 3H), 2.10-2.05 (m, 2 H), 2.06 (s, 3H), 2.04 (s, 3H), 2.02 (s, 3H), 1.53-1.47 (m, 2H), 1.29-1.22 (m, 2H), 0.87 (t, J=7.50 Hz, 3H). ¹³C-NMR (125 MHz, CDCl₃): δ 172.56, 171.57, 170.50, 169.09, 168.63, 72.66, 71.62, 71.46, 61.07, 54.99, 39.68, 36.18, 27.53, 22.12, 21.00, 20.59, 20.57, 20.46, 13.63. HRMS (m/z): C₁₉H₃₀NO₉S (M+H)⁺: Calcd, 448.1636; Found, 448.1640. C₁₉H₂₉NNaO₉S (M+Na)⁺: Calcd, 470.1455; Found, 470.1459. C₁₉H₂₉KNO₅S (M+K)⁺: Calcd, 486.1195; Found, 486.1197.

### Compound 26 and 25: N-(i-Valeryl)-α-D-5-thio-glucosamine (26) and N-6-valeryl)-1,3,4,6-tena-O-acetyl-a-D-5-thio-glucosamine (25)

The title compounds were prepared from the material thus obtained following General Procedures A and B. For 26: ¹H-NMR (500 MHz, D₂O): δ 4.97 (d, J=2.50 Hz, 1H), 4.10 (dd, J=10.0, 3.00 Hz, 1H), 3.93 (dd, J=12.0, 5.50 Hz, 1H), 3.90 (dd, J=12.0, 3.50 Hz, 1H), 3.70 (t, J=9.50 Hz, 1H), 3.66 (t, J=9.50 Hz, 1H), 3.26 (ddd, J=9.50, 5.00, 3.00 Hz, 1H), 2.15 (d, J=7.50 Hz, 2H), 2.03-1.95 (m, 1H), 0.92 (d, J=6.50 Hz, 3H), 0.91 (d, J=6.50 Hz, 3H). ¹³C-NMR (125 MHz, D₂O): δ 176.45, 74.06, 71.84, 71.38, 60.16, 58.01, 44.86, 43.12, 26.20, 21.54, 21.36. HRMS (m/z): C₁₁H₂₂NO₅S (M+H)⁺: Calcd, 280.1213; Found, 280.1201. C₁₁1⁻1₂₁NNaO₅S (M+Na)⁺: Calcd, 302.1033; Found, 302.1022. C₁₁1⁻1₂₁KNO₅S (M+K)⁺: Calcd, 318.0772; Found, 318.0759.

For 25: ¹H-NMR (500 MHz, CDCl₃): δ 5.95 (d, J=3.00 Hz, 1H), 5.71 (d, J=8.50 Hz, 1H), 5.37 (dd, J=10.5, 9.50 Hz, 1H), 5.18 (dd, J=11.0, 10.0 Hz, 1H), 4.65-4.60 (m, 1H), 4.34 (dd, J=12.0, 5.00 Hz, 1H), 4.03 (dd, J=12.0, 3.00 Hz, 1H), 3.47 (ddd, J=11.0, 5.00, 3.00 Hz, 1H), 2.16 (s, 3H), 2.06 (s, 3H), 2.04 (s, 3H), 2.02 (s, 3H), 2.01-1.92 (m, 3H), 0.87 (d, J=6.50 Hz, 6H). ¹³C-NMR (125 MHz, CDCl₃): δ 171.88, 171.66, 170.52, 169.11, 168.62, 72.64, 71.56, 71.50, 61.08, 55.07, 45.77, 39.69, 26.02, 22.20, 22.11, 21.01, 20.67, 20.60, 20.48. HRMS (m/z): C₁₉H₃₀NO₉S (M+H)⁺: Calcd, 448.1636; Found, 448.1641. C₁₉H₂₉NNaO₉S (M+Na)⁺: Calcd, 470.1455; Found, 470.1460. C₁₉H₂₉KNO₅S (M+K)⁺: Calcd, 486.1195; Found, 486.1200.

### Compound 28 and 27: N-Hexonoyl-a-D-5-thio-glucosamine (28) and N-hexonoyl-1,3,4,6-tetra-O-acetyl-a-D-5-thio-glucosamine (27)

The title compounds were prepared from the material thus obtained following General Procedures A and B. For 28: ¹H-NMR (500 MHz, D₂O): δ 4.96 (d, J=2.50 Hz, 1H), 4.10 (dd, J=10.0, 3.00 Hz, 1H), 3.93 (dd, J=12.0, 5.50 Hz, 1H), 3.88 (dd, J=12.0, 3.00 Hz, 1H), 3.70 (t, J=9.50 Hz, 1H), 3.66 (t, J=9.50 Hz, 1H), 3.27 (ddd, J=9.50, 5.50, 3.50 Hz, 1H), 2.28 (t, J=7.50 Hz, 2H), 1.63-1.56 (m, 2H), 1.30-1.27 (m, 4H), 0.86 (t, J=7.00 Hz, 3H). ¹³C-NMR (125 MHz, D₂O): (δ 177.26, 74.05, 71.81, 71.42, 60.18, 58.02, 43.12, 35.66, 30.40, 25.04, 21.65, 13.20. HRMS (m/z): C₁₂H₂₄NO₅S (M+H)⁺: Calcd, 294.1370; Found, 294.1370. C₁₂H₂₃NNaO₅S (M+Na)⁺: Calcd, 316.1189; Found, 316.1190. C₁₂H₂₃KNO₅S (M+K)⁺: Calcd, 332.0929; Found, 332.0927.

For 27: ¹H-NMR (500 MHz, CDCl₃): δ 5.92 (d, J=3.00 Hz, 1H, H-1), 5.72 (d, J=8.50 Hz, 1H, N—H), 5.38 (dd, J=11.0, 10.0 Hz, 1H), 5.18 (dd, J=11.0, 10.0 Hz, 1H), 4.66-4.60 (m, 1H), 4.35 (dd, J=12.0, 5.00 Hz, 1H), 4.01 (dd, J=12.0, 3.00 Hz, 1H), 3.46 (ddd, J=11.0, 5.00, 3.00 Hz, 1H), 2.17 (s, 3H), 2.10-2.05 (m, 2H), 2.06 (s, 3H), 2.03 (s, 3H), 2.02 (s, 3H), 1.55-1.48 (m, 2H), 1.31-1.25 (m, 2H), 1.25-1.17 (m, 2H), 0.85 (t, J=7.50 Hz, 3H). ¹³C-NMR (125 MHz, CDCl₃): δ 172.61, 171.62, 170.59, 169.13, 168.69, 72.65, 71.57, 71.32, 61.00, 54.90, 39.61, 36.46, 31.15, 25.20, 22.28, 21.08, 20.65, 20.51, 13.84. HRMS (m/z): C₂₀H₃₂NO₉S (M+H)⁺: Calcd, 462.1792; Found, 462.1791. C₂₀H₃₅N₂O₉S (M+NH₄)⁺:

Calcd, 479.2058; Found, 479.2063. C₂₀H₃₁NNaO₉S (M+Na)⁺: Calcd, 484.1612; Found, 484.1612. C₂₀H₃₁KNO₅S (M+K)⁺: Calcd, 500.1351; Found, 500.1351.

### Compound 30 and 29: N-Pivaloyl-α-D-5-thio-glucosamine (30) and N-pivaloyl-1, 3,4, 6-tetra-O-acetyl-a-D-5-thio-glucosamine (29)

The title compounds were prepared from the material thus obtained following General Procedures A and B.

For 30: ¹H-NMR (500 MHz, D₂O): δ 4.96 (d, J=3.00 Hz, 1H), 4.13 (dd, J=10.5, 3.00 Hz, 1H), 3.93 (dd, J=12.0, 5.50 Hz, 1H), 3.88 (dd, J=12.0, 3.50 Hz, 1H), 3.76 (t, J=10.0 Hz, 1H), 3.67 (t, J=10.0 Hz, 1H), 3.26 (ddd, J=10.0, 5.50, 3.50 Hz, 1H), 1.19 (s, 9H). ¹³C-NMR (125 MHz, D₂O): δ 182.40, 74.05, 71.75, 71.34, 60.18, 58.16, 43.19, 38.57, 26.47. HRMS (m/z): C₁₁H₂₂NO₅S (M+H)⁺: Calcd, 280.1213; Found, 280.1203. C₁₁H₂₁NNaO₅S (M+Na)⁺: Calcd, 302.1033; Found, 302.1025. C₁₁H₂₁KNO₅S (M+K)⁺: Calcd, 318.0772; Found, 318.0767.

For 29: ¹H-NMR (500 MHz, CDCl₃): δ 6.01 (d, J=3.50 Hz, 1H), 5.90 (d, J=8.50 Hz, 1H), 5.38 (dd, J=10.5, 9.50 Hz, 1H), 5.18 (dd, J=10.5, 10.0 Hz, 1H), 4.58-4.54 (m, 1H), 4.34 (dd, J=12.0, 5.00 Hz, 1H), 4.03 (dd, J=12.0, 3.00 Hz, 1H), 3.48 (ddd, J=11.0, 5.00, 3.00 Hz, 1H), 2.17 (s, 3H), 2.07 (s, 3H), 2.04 (s, 3H), 2.02 (s, 3H), 1.09 (s, 9H). ¹³C-NMR (125 MHz, CDCl₃): δ 177.87, 171.76, 170.54, 169.13, 168.59, 72.24, 71.59, 71,34, 61.14, 55.44, 39.81, 38.58, 27.19, 20.86, 20.62 (2 C), 20.50. HRMS (m/z): C₁₉H₃₀NO₉S (M+H)⁺: Calcd, 448.1636; Found, 448.1636. C₁₉H₂₉NNaO₉S (M+Na)⁺: Calcd, 470.1455; Found, 470.1453. C₁₉H₂₉KNO₅S (M+K)⁺: Calcd, 486.1195; Found, 486.1191.

### Compound 33 and 34: N-(2-Cyclohexylacetyl)-1,3,4,6-tetra-O-acetyl-a-D-5-thio-glucosamine (33) and N-(2-cyclohexylacetyl)-a-D-5-thio-glucosamine (34)

The title compounds were prepared from the material thus obtained following General Procedures D and E.

For 33: ¹H-NMR (600 MHz, CDCl₃): δ 5.95 (d, J=3.00 Hz, 1H), 5.68 (d, J=9.00 Hz, 1H), 5.37 (t, J=10.2 Hz, 1H), 5.18 (t, J=10.2 Hz, 1H), 4.66-4.60 (m, 1H), 4.35 (dd, J=12.0, 4.80 Hz, 1H), 4.04 (dd, J=12.0, 3.00 Hz, 1H), 3.47 (ddd, J=10.8, 4.80, 3.00 Hz, 1H), 2.17 (s, 3H), 2.06 (s, 3H), 2.04 (s, 3H), 2.02 (s, 3H), 1.99 (dd, J=13.8, 7.20 Hz, 1H), 1.92 (dd, J=13.8, 7.20 Hz, 1H), 1.74-1.60 (m, 6H), 1.28-1.20 (m, 2H), 1.19-1.11 (m, 1H), 0.88-0.81 (m, 2H). ¹³C-NMR (150 MHz, CDCl₃): δ 171.81, 171.66, 170.54, 169.12, 168.61, 72.66, 71.54, 71.50, 61.09, 55.06, 44.56, 39.68, 35.20, 32.85, 32.79, 26.02, 25.91, 21.02, 20.67, 20.61, 20.50. HRMS (m/z): C₂₂H₃₄NO₉S (M+H)⁺: Calcd, 488.1949; Found, 488.1951. C₂₂H₃₇N₂O₉S (M+NH₄)⁺: Calcd, 505.2214; Found, 505.2222. C₂₂H₃₃NNaO₉S (M+Na)⁺: Calcd, 510.1768; Found, 510.1765. C₂₂H₃₃KNO₅S (M+K)⁺: Calcd, 526.1508; Found, 526.1456.

For 34: ¹H-NMR (600 MHz, MeOD): δ 4.88 (d, J=2.40 Hz, 1H), 4.10 (dd, J=10.8, 3.00 Hz, 1H), 3.89 (dd, J=11.4, 3.60 Hz, 1H), 3.82 (dd, J=11.4, 6.00 Hz, 1H), 3.67 (dd, J=10.8, 9.0 Hz, 1H), 3.57 (dd, J=10.2, 9.0 Hz, 1H), 3.26 (ddd, J=10.2, 6.00, 4.20 Hz, 1H), 2.11 (d, J=7.20 Hz, 2H), 1.80-1.68 (m, 5H), 1.68-1.64 (m, 1H), 1.32-1.25 (m, 2H), 1.22-1.12 (m, 1H), 1.03-0.96 (m, 2H). ¹³C-NMR (150 MHz, MeOD): δ 175.57, 76.98, 73.65, 73.38, 62.69, 59.88, 45.11, 44.90, 36.89, 34.24, 27.42, 27.33, 27.32. HRMS (m/z): C₁₄H₂₆NO₅S (M+H)⁺: Calcd, 320.1526; Found, 320.1528. C₁₄H₂₅NNaO₅S (M+Na)⁺: Calcd, 342.1346; Found, 342.1347. C₁₄H₂₅KNO₅S (M+K)⁺: Calcd, 358.1085; Found, 358.1042.

### Compound 36 and 35: N-(1-Naphthylacetyl)-α-D-5-thio-glucosamine (36) and N-(1-naphthylacetyl)-1,3,4,6-tetra-O-acetyl-α-D-5-thio-glucosamine (35)

The title compounds were prepared from the material thus obtained following General Procedures A and B. For 36: ¹H-NMR (400 MHz, Acetone-D-6 with some drops of MeOD): δ 8.11 (d, J=8.00 Hz, 1H), 7.86 (d, J=8.00 Hz, 1H), 7.78 (d, J=8.00 Hz, 1H), 7.52-7.38 (m, 4H), 4.87 (d, J=2.40 Hz, 1H), 4.12 (dd, J=10.0, 2.40 Hz, 1H), 4.05 (d, J=2.80 Hz, 2H), 3.86-3.74 (m, 2H), 3.66 (t, J=9.60 Hz, 1H), 3.58 (t, J=9.60 Hz, 1H), 3.23-3.19 (m, 1H). ¹³C-NMR (100 MHz, Acetone-D-6 with some drops of MeOD): δ 173.45, 135.90, 134.40, 134.26, 130.35, 129.73, 129.37, 127.98, 127.53, 127.38, 126.12, 78.00, 74.28, 74.22, 63.63, 60.42, 45.45, 42.13. HRMS (m/z): C₁₈H₂₂NO₅S (M+H)⁺: Calcd, 364.1213; Found, 364.1220. C₁₈H₂₁NNaO₅S (M+Na)⁺: Calcd, 386.1033; Found, 386.1035.

For 35: ¹H-NMR (400 MHz, CDCl₃): δ 7.90-7.80 (m, 3H), 7.56-7.51 (m, 2H), 7.47 (t, J=7.60 Hz, 1H), 7.30 (d, J=6.80 Hz, 1H), 5.83 (d, J=3.20 Hz, 1 H), 5.54 (d, J=8.40 Hz, 1H), 5.25 (t, J=10.4 Hz, 1H), 4.82 (t, J=10.4 Hz, 1H), 4.52-4.47 (m, 1H), 4.26 (dd, J=12.0, 4.80 Hz, 1H), 4.00-3.90 (m, 3H), 3.28 (ddd, J=10.8, 4.80, 3.20 Hz, 1H), 2.02 (s, 3H), 1.94 (s, 3H), 1.67 (s, 3H), 1.66 (s, 3H). ¹³C-NMR (100 MHz, CDCl₃): δ 170.79, 170.45, 170.44, 169.03, 168.17, 133.92, 131.77, 130.25, 128.80, 128.69, 128.34, 127.11, 126.34, 125.66, 123.33, 71.95, 71.28, 70.97, 60.99, 55.30, 41.59, 39.57, 20.55, 20.37, 20.33, 20.03. HRMS (m/z): C₂₆H₃₀NO₉S (M+H)⁺: Calcd, 532.1636; Found, 532.1633. C₂₆H₃₃N₂O₉S (M+NH₄)⁺: Calcd, 549.1901; Found, 549.1887. C₂₆H₂₉NNaO₉S (M+Na)⁺: Calcd, 554.1455; Found, 554.1450. C₂₆H₂₉KNO₅S (M+K)⁺: Calcd, 570.1195; Found, 570.1200.

### Compound 37 and 38: N-[(S)-methylvaleryl]-1,3,4,6-tetra-O-acetyl-α-D-5-thio-glucosamine (37) and N-[(S)-2-methylvaleryl]-a-D-5-thio-glucosamine (38)

The title compounds were prepared from the material thus obtained following General Procedures C and E. For 37: ¹H-NMR (400 MHz, CDCl₃): δ 5.96 (d, J=3.20 Hz, 1H), 5.71 (d, J=8.40 Hz, 1H), 5.38 (t, J=10.8 Hz, 1H), 5.19 (t, J=to 10.8 Hz, 1H), 4.67-4.61 (m, 1H), 4.35 (dd, J=12.0, 5.20 Hz, 1H), 4.04 (dd, J=12.0, 3.20 Hz, 1H), 3.46 (ddd, J=10.8, 4.80, 3.20 Hz, 1H), 2.17 (s, 3H), 2.15-2.12 (m, 2H), 2.07 (s, 3H), 2.04 (s, 3H), 2.03 (s, 3H), 1.75-1.68 (m, 2H), 1.33-1.23 (m, 1H), 1.21-1.10 (m, 1H), 0.88-0.83 (m, 6H). ¹³C-NMR (100 MHz, CDCl₃): δ 172.08, 171.68, 170.54, 169.12, 168.62, 72.67, 71.59, 71.55, 61.11, 55.09, 43.86, 39.72, 32.10, 29.13, 21.02, 20.70, 20.61, 20.50, 18.92, 11.17. HRMS (m/z): C₂₀H₃₂NO₉S (M+H)⁺: Calcd, 462.1792; Found, 462.1793. C₂₀H₃₅N₂O₉S (M+NR₄)⁺: Calcd, 479.2058; Found, 479.2048. C₂₀H₃₁NNaO₉S (M+Na)⁺: Calcd, 484.1612; Found, 484.1609. C₂₀H₃₁KNO₅S (M+K)⁺: Calcd, 500.1351; Found, 500.1319.

For 38: ¹H-NMR (600 MHz, MeOD): δ 4.88 (d, J=2.40 Hz, 1H), 4.11 (dd, J=10.2, 3.00 Hz, 1H), 3.89 (dd, J=11.4, 3.60 Hz, 1H), 3.83 (dd, J=11.4, 6.00 Hz, 1H), 3.68 (dd, J=10.2, 9.0 Hz, 1H), 3.57 (dd, J=10.2, 9.0 Hz, 1H), 3.27 (ddd, J=9.60, 6.00, 4.20 Hz, 1H), 2.24 (dd, J=13.2, 6.0 Hz, 1H), 2.04 (dd, J=13.2, 7.8 Hz, 1H), 1.89-1.83 (m, 1H), 1.44-1.37 (m, 1H), 1.26-1.19 (m, 1H), 0.94 (d, J=6.60 Hz, 3H), 0.92 (t, J=7.20 Hz, 3H). ¹³C-NMR (150 MHz, MeOD): δ 175.83, 76.98, 73.62, 73.38, 62.68, 59.86, 44.90, 44.48, 33.75, 30.49, 19.54, 11.73.

HRMS (m/z): C₁₂H₂₄NO₅S (M+H)⁺: Calcd, 294.1370; Found, 294.1378. C₁₂H₂₃NNaO₅S (M+Na)⁺: Calcd, 316.1189; Found, 316.1193. C₁₂H₂₃KNO₅S (M₊K)⁺: Calcd, 332.0929; Found, 332.0909.

### Compound 39 and 40: N-(5-Methylhexonoyl)-1,3,4,6-tetra-O-acetyl-α-D-5-thio-glucosamine (39) and N-(5-methylhexonoyl)-α-D-5-thio-glucosamine (40)

The title compounds were prepared from the material thus obtained following General Procedures C and E. For 39: ¹H-NMR (400 MHz, CDCl₃): δ 5.95 (d, J=2.80 Hz, 1H), 5.68 (d, J=8.80 Hz, 1H), 5.38 (t, J=10.4 Hz, 1H), 5.18 (t, J=10.4 Hz, 1H), 4.67-4.61 (m, 1H), 4.35 (dd, J=12.0, 5.20 Hz, 1H), 4.04 (dd, J=12.0, 3.20 Hz, 1H), 3.48 (ddd, J=10.8, 4.80, 3.20 Hz, 1H), 2.18 (s, 3H), 2.10-2.00 (m, 2H), 2.07 (s, 3H), 2.04 (s, 3H), 2.03 (s, 3H), 1.56-1.48 (m, 3H), 1.14-1.06 (m, 2H), 0.86 (d, J=6.80 Hz, 6H). ¹³C-NMR (100 MHz, CDCl₃): 65172.57, 171.65, 170.55, 169.12, 168.62, 72.72, 71.72, 71.50, 61.12, 55.07, 39.74, 38.26, 36.77, 27.78, 23.44, 22.41(2 C), 21.06, 20.67, 20.62, 20.50. HRMS (m/z): C₂₁H₃₄NO₉S (M+H)⁺: Calcd, 476.1949; Found, 476.1942. C₂₁H₃₇N₂O₉S (M+NH₄)⁺: Calcd, 493.2214; Found, 493.2189. C₂₁H₃₃NNaO₉S (M+Na)⁺: Calcd, 498.1768; Found, 498.1764. C₂₁H₃₃KNO₅S (M+K)⁺: Calcd, 514.1508; Found, 514.1484.

For 40: ¹H-NMR (600 MHz, MeOD): δ 4.88 (d, J=3.00 Hz, 1H), 4.10 (dd, J=10.2, 3.00 Hz, 1H), 3.89 (dd, J=11.4, 3.60 Hz, 1H), 3.82 (dd, J=11.4, 6.00 Hz, 1H), 3.67 (dd, J=10.2, 9.0 Hz, 1H), 3.58 (dd, J=10.2, 9.0 Hz, 1H), 3.26 (ddd, J=9.60, 6.00, 4.20 Hz, 1H), 2.22 (d, J=7.80 Hz, 2H), 1.65-1.60 (m, 2H), 1.59-1.53 (m, 1H), 1.25-1.21 (m, 2H), 0.89 (t, J=6.60 Hz, 6H). ¹³C-NMR (150 M Hz, MeOD): δ 176.36, 76.93, 73.60, 73.43, 62.68, 59.87, 44.90, 39.64, 37.34, 29.05, 24.92, 22.96. HRMS (m/z): C₁₃H₂₆NO₅S (M+H)⁺: Calcd, 308.1526; Found, 308.1528. C₁₃H₂₅NNaO₅S (M+Na)⁺: Calcd, 330.1346; Found, 330.1349. C₁₃H₂₅KNO₅S (M+K)⁺: Calcd, 346.1085; Found, 346.1049.

### EXAMPLE XII

### Characterization of Analogues

Analogues (9, 10, 17-41) were tested in COS-7 cells, dosed at a final concentration of 50 μM in DMSO for 24 h. Following treatment, cells were washed with PBS, harvested into SDS-PAGE loading buffer and analyzed by western blot using standard procedures; blots were probed with the anti-O-GlcNAc antibody (CTD 110.6).

Compounds 17, 19, 21, 23, 25, 27, 28, 31, 33, 34, 35, 36, 37, 38, and 41 decreased O-GlcNAc levels in cells at a dose of 50 μM, with the hexyl (2728) and 3-methyl pentyl (3738) substituents showing the greatest promise at decreasing O-GlcNac levels to a comparable extent to the parent compound Ac-5SGlcNAc 9. Representative western blots are shown in FIG. 7.

### EXAMPLE XIII

### Animal Studies

Ac-5SGlcNAc 9 and the deprotected analogue with the hexyl substituent at the C2 position 28 have been analyzed for effectiveness at decreasing O-GlcNAc levels in mice. Pilot studies were conducted with mice dosed in triplicate with either compound or vehicle alone. Compounds were dosed at 300 mg/kg intraperitoneally using 75% DMSO as vehicle for delivery of 9 and phosphate-buffered saline (PBS) for delivery of 28. Mice were euthanized using a CO₂ chamber after 16 hours exposure to either compound, and tissues (liver, lung, heart, kidney, spleen, pancreas, brain, blood, fat and muscle) harvested and flash frozen in liquid nitrogen. Frozen tissues were ground using a pestle and mortar, and resuspended in cell lysis buffer (50 mM NaH₂PO₄, 150 mM NaCl, 0.1% SDS, 1% NP-40, 0.25% sodium deoxycholate, 1 mM EDTA, 30 mM sodium fluoride, 30 mM β-glycerophosphate, 5 mM sodium pyrophosphate, 1 mM Thiamet-G, protease inhibitor cocktail (Roche)) at a concentration of 100 mg tissue1 mL buffer prior to homogenization. Homogenates were centrifuged at 13,000 rpm for 20 min, the supernatant removed, added to 2×SDS-PAGE loading buffer and boiled for 10 mM. SDS-PAGE and western blots were performed using standard procedures. Blots were probed using anti-O-GlcNAc antibody (CTD110.6, Santa Cruz Biotechnology) and anti-actin antibody for a loading control.

Compounds 9 and 28 were both effective at decreasing O-GlcNAc levels during the 16 hour dosing period in the liver, lung, heart, kidney, spleen, pancreas, fat and muscle. Representative western blots are shown in FIG. 8.

### EXAMPLE XIV

### Chronic Lymphoid Leukaemia (CLL) Cells

Primary CLL cells, obtained from Dr. David Spaner at the University of Toronto, were tested for their response to Ac-5SGlcNAc (9). Cells were plated at a confluency of 1 million cells/mL, and treated with between 0-250 μM 9, in the presence or absence of cytokine (1000 U/mL IL-2 and 1 μg/mL Resiquimod) stimulation to mimic cancer progression. Cells were observed over the course of 5 days, and harvested after this time into SDS-PAGE loading buffer before being subjected to western blotting.

At concentrations of 50 μM Ac-5SGlcNAc and higher in stimulated cells, the proliferation that was obvious in untreated stimulated cells was absent. Ac-5SGlcNAc was not toxic to either unstimulated or stimulated cells, but instead appeared to prevent proliferation of stimulated cells. Western blots (FIG. 9) showed that O-GlcNAc levels were decreased in a dose-dependent fashion.

### EXAMPLE XV

### Sialyl-lewis X (sLe^{X}) Expression in HepG2 Cells

5-T-Fuc and/or Ac-5-T-Fuc were tested to determine whether these compounds would inhibit intracellular fucose transferases. A dose-dependent decrease in the level of the tetrasaccharide glycan sialyl-Lewis^{X} (sLe^{x}) was found in the hepatyocyte cell line HepG2, indicating the use of this compound in treatment or prevention of inflammation.

More specifically, monolayers of Hep G2 cells were grown to about 75% confluency in DMEM containing 10% FBS before they were switched to serum free media for 10 h. FBS-free conditions were utilized to limit the amount of fucose which could be made available to cells upon degradation of serum glycoproteins. Cells were subsequently exposed to peracetylated 5-thio-L-fucose (5-T-Fuc) in serum free media for 48 h after which the media were collected, gently centrifuged (800 rpm, 10 min, 4° C.) to remove dead cells, and concentrated (˜10-fold) by centrifugal filration (Amicon, NMWCO 3000 daltons). Cells were washed with cold PBS prior to harvesting them in PBS+0.5% SDS. Proteins were solubilised upon sonication (2 blasts, 10 sec, 15% power) and protein concentrations were determined for all samples by the DC protein concentration assay (BioRad). Samples were normalized prior to analysis. Identical amounts of proteins were analyzed for all lectin-blotting experiments. Cell extracts and conditioned media containing secreted proteins were all analyzed on 8% polyacrylamide gels (FIG. 10).

5-T-Fuc caused a significant, dose-dependent decrease in sLe^{X} expression on proteins obtained from treated cells (FIG. 10A) though no decrease in Aleura aurantia lectin reactivity was observed (FIG. 10B). This suggests that not all fucosylated antigens are affected by 5-T-Fuc. In contrast, there was dose-dependent increase in Maackia amurensis lectin binding in 5-T-Fuc-treated cells (FIG. 10C) which suggests that this inhibitor may allow for an increase in the rate of formation of sialylated oligosaccharides by limiting the activity of competing fucosyltransferases. Monolayers of HepG2 cells were exposed to 50 μM 5-T-Fuc for varying lengths of time before they were harvested as described above. When necessary, fresh media and inhibitors were added after 24 h. Alternatively, cells were exposed to 5-T-Fuc for 24 h after which the media was replaced and cells were harvested at the indicated time-points (FIG. 11).

CHO K1 cells exposed to 5-T-Fuc demonstrate a “switch-like” response in their expression of core-fucosylated antigens as assessed by lectin-blotting with the Aleura aurantia lectin: no further decrease in lectin reactivity was observed above inhibitor concentrations of 5 μM (FIG. 12). Cells were exposed to 5-T-Fuc for 48 h prior to harvesting as described above.

The present invention has been described with regard to one or more embodiments. However, it will be apparent to persons skilled in the art that a number of variations and modifications can be made without departing from the scope of the invention as defined in the claims.

### REFERENCES

¹ Varki, A. and Lowe, J. B., in Essentials of Glycobiology, edited by A. Varki et al. (CSH Press, 2009).
² Torres, C. R. and Hart, G. W., Topography and polypeptide distribution of terminal N-acetylglucosamine residues on the surfaces of intact lymphocytes. Evidence for O-linked GlcNAc. J Biol Chem 259, 3308 (1984).
³ Hart, G. W., Housley, M. P., and Slawson, C., Cycling of O-linked beta-N-acetylglucosamine on nucleocytoplasmic proteins. Nature 446, 1017 (2007).
⁴ Lyons, S. D., Sant, M. E., and Christopherson, R. I., Cytotoxic mechanisms of glutamine antagonists in mouse L1210 leukemia. J Biol Chem 265 (19), 11377 (1990).
⁵ Lenzen, S. and Panten, U., Alloxan: history and mechanism of action. Diabetologia 31 (6), 337 (1988).
⁶ Gross, B. J., Kraybill, B. C., and Walker, S., Discovery of O-GlcNAc transferase inhibitors. J Am Chem Soc 127 (42), 14588 (2005).
⁷ Caldwell, S. A. et al., Nutrient sensor O-GlcNAc transferase regulates breast cancer tumorigenesis through targeting of the oncogenic transcription factor FoxM1. Oncogene (2010).
⁸ Varki, A. et al., Essentials of Glycobiology, 2nd edition ed. (CSH Press, 2009).
⁹ Vocadlo, D. J. et al., A chemical approach for identifying O-GlcNAc-modified proteins in cells. Proc Natl Acad Sci USA 100 (16), 9116 (2003).
¹⁰ Marshall, S., Nadeau, O., and Yamasaki, K., Dynamic actions of glucose and glucosamine on hexosamine biosynthesis in isolated adipocytes: differential effects on glucosamine 6-phosphate, UDP-N-acetylglucosamine, and ATP levels. J Biol Chem 279 (34), 35313 (2004).
¹¹ Altschuler, Y. et al., Clathrin-mediated endocytosis of MUCl is modulated by its glycosylation state. Mol Biol Cell 11 (3), 819 (2000).
¹² Luchansky, S. J., Argade, S., Hayes, B. K., and Bertozzi, C. R., Metabolic functionalization of recombinant glycoproteins. Biochemistry 43 (38), 12358 (2004).
¹³ Prescher, J. A., Dube, D. H., and Bertozzi, C. R., Chemical remodelling of cell surfaces in living animals. Nature 430 (7002), 873 (2004).
¹⁴ Park, D. et al., Characterization and role of fucose mutarotase in mammalian cells. Glycobiology 17 (9), 955 (2007).
¹⁵ Yurchenco, P. D. and Atkinson, P. H., Equilibration of fucosyl glycoprotein pools in HeLa cells. Biochemistry 16 (5), 944 (1977).
¹⁶ Saxon, E. et al., Investigating cellular metabolism of synthetic azidosugars with the Staudinger ligation. J Am Chem Soc 124 (50), 14893 (2002).

¹⁷ Hang, H. C., Yu, C., Kato, D. L., and Bertozzi, C. R., A metabolic labeling approach toward proteomic analysis of mucin-type O-linked glycosylation. Proc Natl Acad Sci USA 100 (25), 14846 (2003).
¹⁸ Sampathkumar, S. G. et al., Metabolic installation of thiols into sialic acid modulates adhesion and stem cell biology. Nat Chem Biol 2 (3), 149 (2006).
¹⁹ Chang, P. V. et al., Metabolic labeling of sialic acids in living animals with alkynyl sugars. Angew Chem Int Ed Engl 48 (22), 4030 (2009).
²⁰ Chang, P. V. et al., Copper-free click chemistry in living animals. Proc Nall Acad Sci USA 107 (5), 1821 (2010).
²¹ Sletten, E. M. and Bertozzi, C. R., Bioorthogonal chemistry: fishing for selectivity in a sea of functionality. Angew Chem Int Ed Engl 48 (38), 6974 (2009).
²² Hasegawa, A., Kawai, Y., Kasugai, H., and Kiso, M., Studies on Hetero Sugars .1. Synthesis of 2-Acetamido-2-Deoxy-5-Thio-D-Glucopyranose. Carbohydrate Research 63 (June), 131 (1978).
²³ Tanahashi, E., Kiso, M., and Hasegawa, A., Studies on Hetero Sugars .8. Synthesis of 2-Acetamido-2-Deoxy-5-Thio-Alpha-D-Galactopyranose. Journal of Carbohydrate Chemistry 2 (2), 129 (1983).
²⁴ Hashimoto, H. and Izumi, M., Efficient and Stereoselective 1,2-cis Glycoside Formation of 5-Thioaldopyranoses: Glycosylation with Peracetylated 5-Thio-Darabinopyranosyl and 5-Thio-L-fucopyranosyl Trichloroacetimidates. Tet Lett 34, 4949 (1993).
²⁵ Nestadt, B. and Vanes, T., Derivatives of 5-Deoxy-5-Thio-D-Xylose. Tetrahedron 24 (4), 1973 (1968).
²⁶ Vanes, T. and Whistler, R. L., Derivatives of 5-Deoxy-5-Seleno-D-Xylose. Tetrahedron 23 (6), 2849 (1967).
²⁷ Tanahashi, E., Kiso, T., and Hasegawa, A., A facile synthesis of 2-acetamido-2-deoxy-5-thio-D-glucopyranose. Carbohydr Res 117, 304 (1983).
²⁸ Bock, K., Ogawa, S., and Orihara, M., Proton and C-13 NMR-Spectra of 6 Pseudo-2-Acetamido-2-Deoxy-D-Hexopyranoses. Carbohydrate Research 191 (2), 357 (1989).
²⁹ Offen, W. et al., Structure of a flavonoid glucosyltransferase reveals the basis for plant natural product modification. EMBO J 25 (6), 1396 (2006).
³⁰ Tsuruta, O., Shinohara, G., Yuasa, H., and Hashimoto, H., UDP-N-acetyl-5-thio-galactosamine is a substrate of lactose synthase. Bioorg Med Chem Lett 7 (19), 2523 (1997).
³¹ Tsuruta, 0. et al., Synthesis of GDP-5-thiosugars and their use as glycosyl donor substrates for glycosyltransferases. J Org Chem 68 (16), 6400 (2003).
³² Seo, K. C. et al., Chemoenzymatic syntheses of carbasugar analogues of nucleoside diphosphate sugars: UDP-carba-Gal, UDP-carba-GlcNAc, UDP-carba-Glc, and GDP-carba-Man. Chem Commun (13), 1733 (2009).
³³ Eklund, E. A. et al., Hydrophobic Man-1-P derivatives correct abnormal glycosylation in Type I congenital disorder of glycosylation fibroblasts. Glycobiology 15 (11), 1084 (2005).
³⁴ Hinderlich, S. et al, Molecular cloning and characterization of murine and human N-acetylglucosamine kinase. Eur J Biochem 267 (11), 3301 (2000).
³⁵ Mio, T., Yamada-Okabe, T., Arisawa, M., and Yamada-Okabe, H., Functional cloning and mutational analysis of the human cDNA for phosphoacetylglucosamine mutase: identification of the amino acid residues essential for the catalysis. Biochim Biophys Acta 1492 (2-3), 369 (2000).
³⁶ Bourgeaux, V., Piller, F., and Piller, V., Two-step enzymatic synthesis of UDP-N-acetylgalactosamine. Bioorg Med Chem Lett 15 (24), 5459 (2005).
³⁷ Leiting, B., Pryor, K. D., Eveland, S. S., and Anderson, M. S., One-day enzymatic synthesis and purification of UDP-N-[1-14C]acetyl-glucosamine. Anal Biochem 256 (2), 185 (1998).
³⁸ Rabina, J. et al., Analysis of nucleotide sugars from cell lysates by ion-pair solid-phase extraction and reversed-phase high-performance liquid chromatography. Glycoconj J 18 (10), 799 (2001).
³⁹ Chien, R. L. and Burgi, D. S., Field-Amplified Polarity-Switching Sample Injection in High-Performance Capillary Electrophoresis. Journal of Chromatography 559 (1-2), 153 (1991).

⁴⁰ Martinez-Fleites, C. et al., Structure of an O-GlcNAc transferase homolog provides insight into intracellular glycosylation. Nat Struct Mol Biol 15 (7), 764 (2008).
⁴¹ Leatherbarrow, R. J., GraFit Version 5. (Erithacus Software Ltd., Horley, UK, 2001).
⁴² Macauley, M. S., Stubbs, K. A., and Vocadlo, D. J., O-GlcNAcase catalyzes cleavage of thioglycosides without general acid catalysis. J Am Chem Soc 127 (49), 17202 (2005).
⁴³ Dennis, R. J. et al., Structure and mechanism of a bacterial beta-glucosaminidase having O-GlcNAcase activity. Nat Struct Mol Biol 13 (4), 365 (2006).
⁴⁴ Tomiya, N. et al., Determination of nucleotides and sugar nucleotides involved in protein glycosylation by high-performance anion-exchange chromatography: sugar nucleotide contents in cultured insect cells and mammalian cells. Anal Biochem 293 (1), 129 (2001).
⁴⁵ Tumock, D. C. and Ferguson, M. A., Sugar nucleotide pools of Trypanosoma brucei, Trypanosoma cruzi, and Leishmania major. Eukaryot Cell 6 (8), 1450 (2007).
⁴⁶ Feng, H. T., Wong, N., Wee, S., and Lee, M. M., Simultaneous determination of 19 intracellular nucleotides and nucleotide sugars in Chinese Hamster ovary cells by capillary electrophoresis. J Chromatogr B Analyt Technol Biomed Life Sci 870 (1), 131 (2008).
⁴⁷ Hasegawa, A and Kiso, M, A Simple Preparation of 2-(Acylamino)-2-Deoxy-3,4:5,6-Di-O-Isopropyladine-adehydo-_{D}-Glucose Dimethyl and Dibenzylacetal. Carbohydr Res 79, 265 (1980).
⁴⁸ Damager, I. et al., Synthesis and characterisation of novel chromogenic substrates for human pancreatic alpha-amylase. Carbohydr Res 339 (10), 1727 (2004).
⁴⁹ Baudry, M. et al., Synthetic studies in the 5-thio-D-xylopyranose series part 2: Coupling of 5-thio-D-xylopyranosyl donors with electron-rich aryl moieties: Access to C-aryl 5-thio-D-xylopyranosides. Tet Lett 54, 7447 (1998).
⁵⁰ Tsuruta, O. et al., Affinity of 5-thio-L-fucose-containing Lewis X (LeX) trisaccharide analogs to anti-LeX monoclonal antibody. Bioorg Med Chem Lett 9 (7), 1019 (1999).
⁵¹ Yuasa, H., Suga, Y., and Hashimoto, H., Glycosidation Reactions of 5-Thioxylpyranosyl Donors. Lett Org Chem 5 (6), 429 (2008).
⁵² Davis, L. I. and Blobel, G., Nuclear pore complex contains a family of glycoproteins that includes p62: glycosylation through a previously unidentified cellular pathway. Proc Natl Acad Sci USA 84 (21), 7552 (1987).
⁵³ Lubas, W. A., Smith, M., Starr, C. M., and Hanover, J. A., Analysis of nuclear pore protein p62 glycosylation. Biochemistry 34 (5), 1686 (1995).
⁵⁴ Khidekel, N. et al., A chemoenzymatic approach toward the rapid and sensitive detection of O-GlcNAc posttranslational modifications. J Am Chem Soc 125 (52), 16162 (2003).
⁵⁵ Saxon, E. and Bertozzi, C. R., Cell surface engineering by a modified Staudinger reaction. Science 287 (5460), 2007 (2000).
⁵⁶ Greig, I. R., Macauley, M. S., Williams, I. H., and Vocadlo, D. J., Probing synergy between two catalytic strategies in the glycoside hydrolase O-GlcNAcase using multiple linear free energy relationships. J Am Chem Soc 131 (37), 13415 (2009).
⁵⁷ Boehmelt, G. et al., Decreased UDP-GlcNAc levels abrogate proliferation control in EMeg32-deficient cells. EMBO J19 (19), 5092 (2000).
^{58.} Driguez, H. & Henrissat, B. A novel synthesis of 5-thio-D-glucose. Tet Lett 22, 5061-5062 1981).
^{59.} Tsuda, Y. et al. Thio Sugars. I. Radical promoted thione-thiol rearrangement of cyclic thionocarbonates: synthesis of 5-thioglucose. Chem Pharm Bull 44, 1465-1475 (1996).
⁶⁰ Csuk, R. & Glanzer, B. I. A short synthesis of 2-acetimido-2-deoxy-5-thio-D-glucose and D-mannose from 5-thio-glucal. J Chem Soc Chem Comm, 343-344 (1986).
^{61.} Taggi, A. E. et al. The development of the first catalyzed reaction of ketenes and imines: catalytic, asymmetric synthesis of beta-lactams. J Am Chem Soc 124, 6626-6635 (2002).
^{62.} Bedard, P. W. & Kaila, N. Selectin inhibitors: a patent review. Expert Opin Ther Pat 20, 781-93 (2010).
^{63.} Lowe, J. B. Glycan-dependent leukocyte adhesion and recruitment in inflammation. Curr Opin Cell Biol 15, 531-8 (2003).
^{64.} Sperandio, M., Gleissner, C. A. & Ley, K. Glycosylation in immune cell trafficking. Immunol Rev 230, 97-113 (2009).
^{65.} Terraciano, A. J. et al. Sialyl Lewis X, Lewis X, and N-acetyllactosamine expression on normal and glaucomatous eyes. Curr Eye Res 18, 73-8 (1999).
^{66.} Miyoshi, E., Moriwaki, K. & Nakagawa, T. Biological function of fucosylation in cancer biology. J Biochem 143, 725-9 (2008).
^{67.} Moriwaki, K. & Miyoshi, E. Fucosylation and gastrointestinal cancer. World J Hepatol 2, 151-61 (2010).
^{68.} Barthel, S. R. et al. Alpha 1,3 fucosyltransferases are master regulators of prostate cancer cell trafficking. Proc Natl Acad Sci USA 106, 19491-6 (2009).
^{69.} Cai, Y. H. et al. Abrogation of functional selectin-ligand expression reduces migration of pathogenic CD8+ T cells into heart. J Immunol 176, 6568-75 (2006).
^{70.} Gitlin, J. M. et al. Disruption of tissue-specific fucosyltransferase VII, an enzyme necessary for selectin ligand synthesis, suppresses atherosclerosis in mice. Am J Pathol 174, 343-50 (2009).
^{71.} Maly, P. et al. The alpha(1,3)fucosyltransferase Fuc-TVII controls leukocyte trafficking through an essential role in L-, E-, and P-selectin ligand biosynthesis. Cell 86, 643-53 (1996).
^{72.} Weninger, W. et al. Specialized contributions by alpha(1,3)-fucosyltransferase-IV and FucT-VII during leukocyte rolling in dermal microvessels. Immunity 12, 665-76 (2000).
^{73.} Malphettes, L. et al. Highly efficient deletion of FUT8 in CHO cell lines using zinc-finger nucleases yields cells that produce completely nonfucosylated antibodies. Biotechnol Bioeng 106, 774-83 (2010).
^{74.} Ma B, Simala-Grant JL, Taylor DE. Fucosylation in prokaryotes and eukaryotes. Glycobiology 2006; 16(12):158R-184R
^{75.} Myers and Miller, CABIOS, 1989, 4:11-17
^{76.} Ausubel et al., Current Protocols in Molecular Biology, John Wiley & Sons, New York, N.Y., 1998.
^{77.} Gennaro, Alfonso Remington: the Science & Practice of Pharmacy by, 20^{th} ed., Williams & Wilkins, (2000).

All citations are hereby incorporated by reference.

1. A method of inhibiting a glycosyltransferase (GT), or of reducing the level of a nucleotide sugar-modified biomolecule, or of treating a condition that is modulated by a GT, in a subject in need thereof, the method comprising administering to the subject an effective amount of a compound of any one of Formula (I-X) or a pharmaceutically acceptable salt thereof: 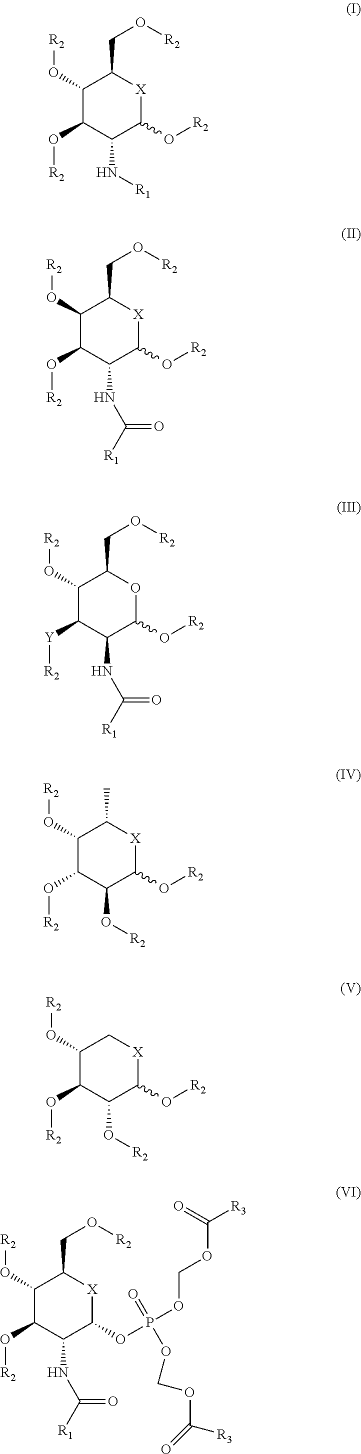 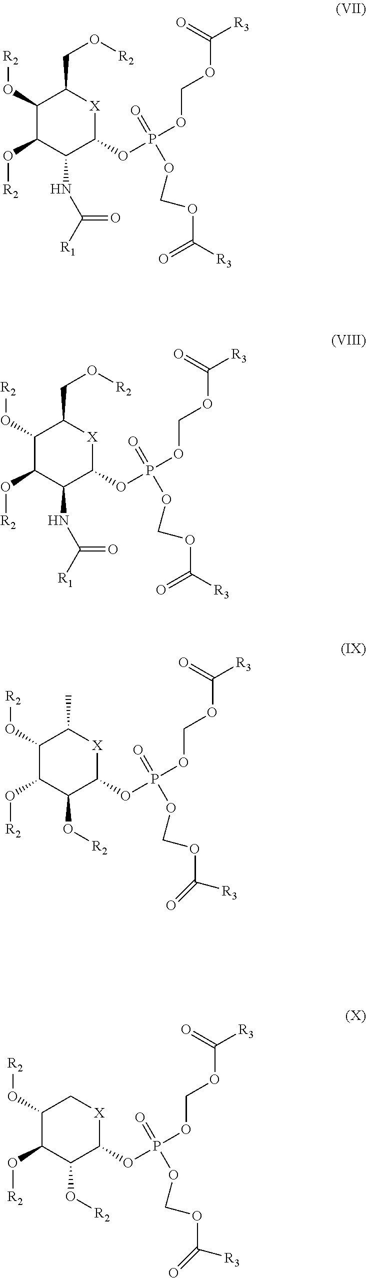
wherein:
X where present is S, Se or CH₂;
Y where present is S or Se;
R₁ is either H or C(O)R₄, wherein R₄ is H, CH₂OH, CH₂N₃, CH₂SH, small branched or unbranched alkyl, ether, thioether, urea, or thiourea, wherein the alkyl, ether, thioether, urea, or thiourea are optionally substituted;
R₂ is H or C(O)R₅, wherein R₅ is optionally substituted alkyl, alkenyl, alkynyl, aryl, or heteroaryl;
R₃ where present is H or C(O)R₆, wherein R₆ is optionally substituted alkyl, alkenyl, alkynyl, aryl, or heteroaryl; and [symbol]
may be the alpha anomer, the beta anomer, or both.

2-3. (canceled)

4. The method of claim 1 wherein the subject is a human.

5. A method of inhibiting a GT in a cell or tissue, or of reducing the level of a nucleotide sugar-modified biomolecule in a sample, the method comprising contacting the cell or tissue or sample with an effective amount of a compound of any one of Formula (I-X) or a pharmaceutically acceptable salt thereof: 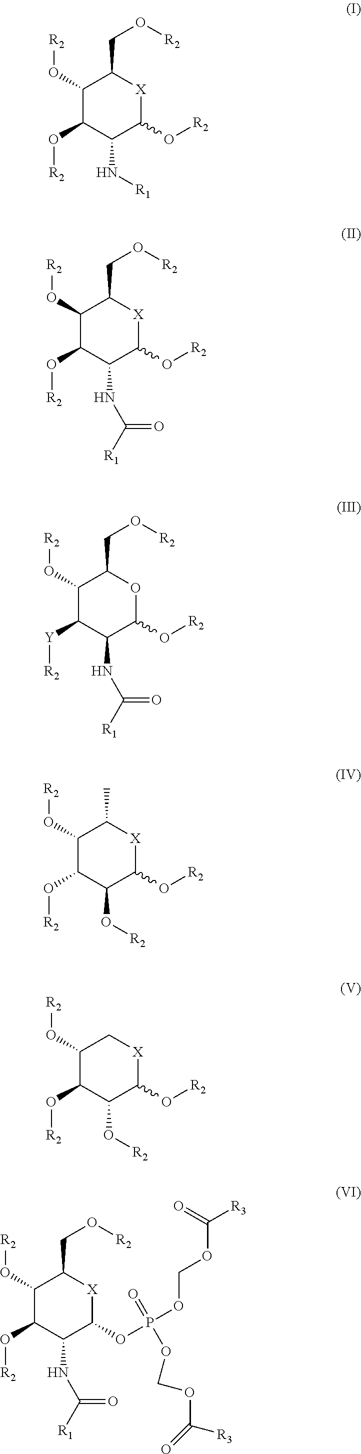 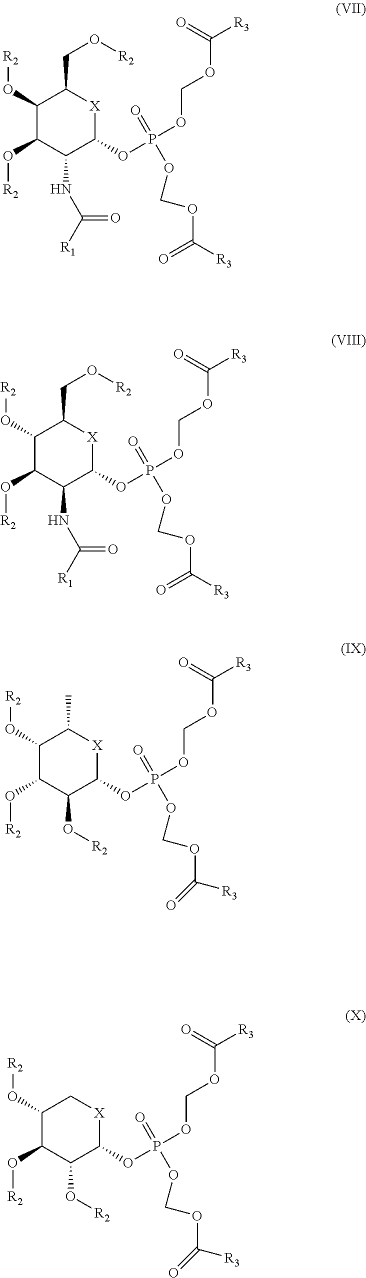
wherein:
X where present is S, Se or CH₂;
Y where present is S or Se;
R₁ is either H or C(O)R₄, wherein R₄ is H, CH₂OH, CH₂N₃, CH₂SH, small branched or unbranched alkyl, ether, thioether, urea, or thiourea, wherein the alkyl, ether, thioether, urea, or thiourea are optionally substituted;
R₂ is H or C(O)R₅, wherein R₅ is optionally substituted alkyl, alkenyl, alkynyl, aryl, or heteroaryl;
R₃ where present is H or C(O)R₆, wherein R₆ is optionally substituted alkyl, alkenyl, alkynyl, aryl, or heteroaryl; and [symbol]
is the alpha anomer, the beta anomer, or both.

6. The method of claim 1 wherein the small alkyl is methyl, ethyl, propyl, butyl, isopropyl, isobutyl, valeryl, or isovaleryl.

7. The method of claim 1 wherein the ether is O-methyl, 0-ethyl, O-propyl, O-isopropyl, O-butyl, or O-isobutyl.

8. The method of claim 1 wherein the thioether is S-methyl, S-ethyl, S-propyl, S-isopropyl, S-butyl, or S-isobutyl.

9. The method of claim 1 wherein one, two, three or four of R₂ is C(O)CH₃.

10. The method claim 1 wherein the GT is a fucosyltransferase (FUT), and the method comprises administering an effective amount of a compound of Formula (IV) or (IX) or a pharmaceutically acceptable salt thereof.

11. The method of claim 5 wherein the GT is a fucosyltransferase (FUT), and the method comprises contacting the sample comprising an antibody or comprises contacting the cell or tissue, with an effective amount of a compound of Formula (IV) or (IX) or a pharmaceutically acceptable salt thereof.

12. (canceled)

13. The method of claim 1 wherein the condition is diabetes, complications of diabetes, inflammation, an autoimmune disorder or cancer.

14. (canceled)

15. The method of claim 1 wherein the compound is 5-T-Fuc.

16. The method of claim 1 wherein the GT is uridine diphospho-N-acetylglucosamine:polypeptide β-N-acetylglucosaminyltransferase (OGT), and the method comprises administering an effective amount of a compound of Formula (I) or (VI) or a pharmaceutically acceptable salt thereof

17-22. (canceled)

23. The method of claim 1 wherein:
X is S,
R₂ is H or C(O)CH₃, and
R₄ is H, CH₂N₃, CH₃, CH₂CH₃, (CH₂)₂CH₃, CH(CH₃)₂, (CH₂)₃CH₃, CH₂CH(CH₃)₂, (CH₂)₄CH₃, C(CH₃)₃, CH₂C₆Hᵢᵢ, CH₂C₁₀H₈, CH₂CH(CH₃)CH₂CH₃, or (CH₂)₃CH(CH₃)₂.

24-33. (canceled)

34. A compound of Formula (I-X) or a pharmaceutically acceptable salt thereof: 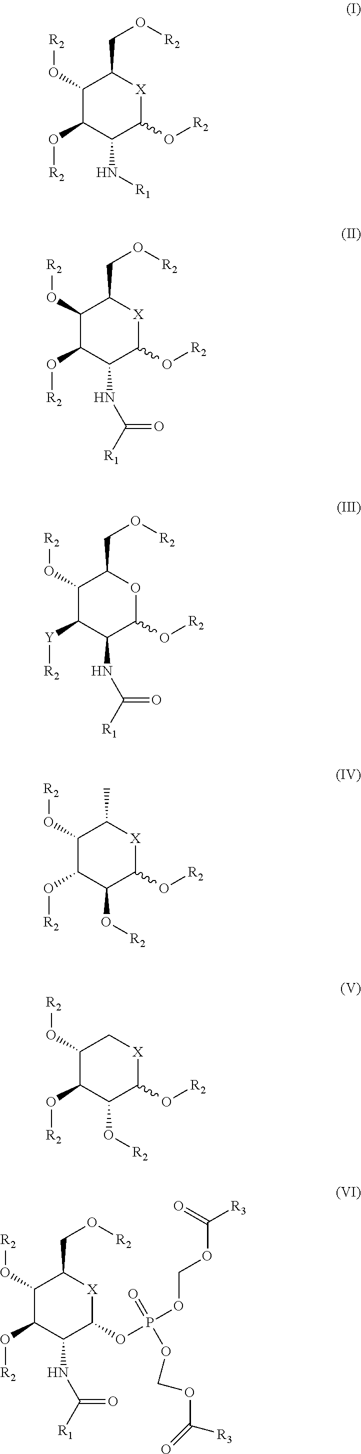 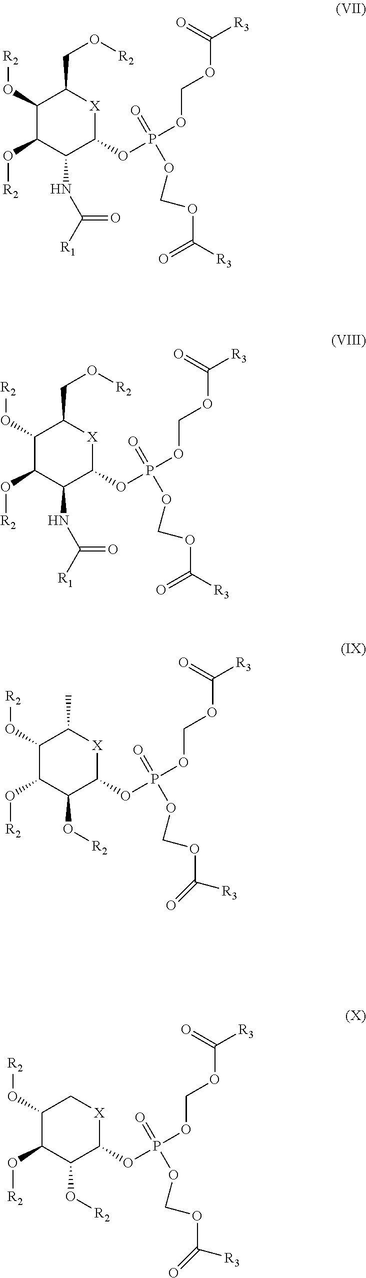
wherein:
X where present is S, Se or CH₂;
Y where present is S or Se;
R₁ is either H or C(O)R₄, wherein R₄ is H, CH₂OH, CH₂N₃, CH₂SH, small branched or unbranched alkyl, ether, thioether, urea, or thiourea, wherein the alkyl, ether, thioether, urea, or thiourea are optionally substituted;
R₂ is H or C(O)R₅, wherein R₅ is optionally substituted alkyl, alkenyl, alkynyl, aryl, or heteroaryl;
R₃ where present is H or C(O)R₆, wherein R₆ is optionally substituted alkyl, alkenyl, alkynyl, aryl, or heteroaryl; and [symbol]
may be the alpha anomer, the beta anomer, or both,
wherein optionally said compound is not 5SGlcNAc, Ac-5SGlcNAc, 5CH₂GlcNAc, UDP-5CH₂GlcNAc, 5SGlcNAz, Ac-5SGlcNAz, or 5-T-Fuc.

35. A pharmaceutical composition comprising the compound of claim 34 or a pharmaceutically acceptable salt thereof, in combination with a pharmaceutically acceptable carrier.

36. The compound of claim 34 wherein the small alkyl is methyl, ethyl, propyl, butyl, isopropyl, isobutyl, valeryl, or isovaleryl.

37. The compound of claim 34 wherein the ether is O-methyl, O-ethyl, O-propyl, O-isopropyl, O-butyl, or O-isobutyl.

38. The compound of claim 34 wherein the thioether is S-methyl, S-ethyl, S-propyl, S-isopropyl, S-butyl, or S-isobutyl.

39. The compound of claim 34 wherein one, two, three or four of R₂ is C(O)CH₃.

40. The compound of claim 34 wherein:
X is S,
R₂ is H or C(O)CH₃, and
R₄ is H, CH₂N₃, CH₃, CH₂CH₃, (CH₂)₂CH₃, CH(CH₃)₂, (CH₂)₃CH₃, CH₂CH(CH₃)₂, (CH₂)₄CH₃, C(CH₃)₃, CH₂C₆Hᵢᵢ, CH₂C₁₀H₈, CH₂CH(CH₃)CH₂CH₃, or (CH₂)₃CH(CH₃)₂.

41. (canceled)
